(19) United States
(12) Patent Application Publication
(10) Pub. No.: US 2026/0298954 A1
(43) Pub. Date: Oct. 1, 2026

(54) SYSTEM, METHOD AND GRAPHICAL USER INTERFACE FOR MANAGING QUALITY CONTROL FOR A DIAGOSTIC SYSTEM

(71) Applicant: Roche Molecular Systems, Inc., Pleasanton, CA (US)

(72) Inventors: Michael Thomas Nguyen, Carlsbad, CA (US); Connor Craig Fitch, Vista, CA (US)

(73) Assignee: Roche Molecular Systems, Inc., Pleasanton, CA (US)

(21) Appl. No.: 19/228,375

(22) Filed: Jun. 4, 2025

Related U.S. Application Data
(63) Continuation of application No. PCT/EP2023/084971, filed on Dec. 8, 2023.
(60) Provisional application No. 63/431,502, filed on Dec. 9, 2022.

(51) Int. Cl. G01N 35/00

(52) U.S. Cl. CPC G01N 35/00623; G01N 2035/00881; G01N 2035/009; G01N 2035/0091

(57) ABSTRACT

A system, a method, and a user interface are described for customizing external quality control testing for a diagnostic system. Quality control assays must be run for each assay panel capable of being run on a diagnostic instrument. Each quality control assay for each assay panel is given a desired frequency and a desired rotation. A QC rule customization module allows a user to create, manage, delete, or view QC rules. Reminders can be sent for upcoming quality control assays that are due. Alerts are sent if the quality control assays are missed.

### CROSS-REFERENCE TO RELATED APPLICATIONS

This application is a continuation application of International Patent Application Serial No. PCT/EP2023/084971, filed Dec. 8, 2023, which claims benefit of U.S. Provisional Patent Application Ser. No. 63/431,502, filed Dec. 9, 2022, each of which is hereby incorporated by reference in its entirety.

### FIELD OF THE INVENTION

The invention relates to an improved system architecture and method for managing quality control for a diagnostic instrument.

### BACKGROUND OF THE INVENTION

Preventable medical errors are now the third leading cause of death in the United States, at more than 250,000 per year. Some rapid molecular diagnostic systems, like GenMark's e-Plex system, are designed to reduce medical error. In order to ensure that a diagnostic system is performing properly, quality control tests should be performed.

If a diagnostic instrument is not performing properly, preventable medical errors can arise. If the instrument cannot be used, the laboratory cannot process samples. In some cases, failure to process samples can be deadly, such as for sample processing cartridges that detect organisms that cause sepsis. Recent studies have shown that patients with severe sepsis or septic shock showed a 7.6% increased likelihood of death for every hour in which antibiotic therapy is not applied. Liang et al., Empiric Antimicrobial Therapy in Severe Sepsis and Septic Shock: Optimizing Pathogen Clearance, Curr Infect Dis Rep. 2015 July; 17(7): 493. Further, when a laboratory cannot process samples, cartridges may expire. Consistently supplying laboratories and hospitals with cartridges for processing patient samples is difficult because of their short shelf-life and the seasonable demand for some tests. For example, during the unprecedented Corona Virus pandemic of 2019-2020, many hospitals and laboratories were at critically low levels of rapid molecular respiratory cartridges. Thus, there is a need to ensure that a diagnostic system is performing properly by running quality control tests.

### SUMMARY OF THE INVENTION

Quality control is designed to detect, reduce, and correct deficiencies in a laboratory's internal analytical process prior to the release of patient results. A quality control scheme should minimize reporting of erroneous results, but not result in excessive repetition of QC Runs.

The first step in the design of a QC system is to define a procedure for Quality Control Testing. Quality Control Tests can be run at the beginning of each shift, after an instrument is serviced, when reagent lots are changed, after calibration, and when patient results seem inappropriate. Keeping track of when Quality Control Tests should be run is complicated, especially when multiple Quality Control Tests must be run for each assay panel capable of being run on a diagnostic instrument. Further, each laboratory has different QC requirements based on workflow patterns, environmental conditions, and operator characteristics.

A QC framework should contain more than just an articulation that a quality control test needs to be run. Laboratories must develop an overall QC strategy that provides a comprehensive plan to ensure that QC runs are not missed or needlessly duplicated. Problems with conventional QC processes are that if a specific QC assay is skipped, there is no mechanism in place to ensure it is run after it has fallen out of rotation.

Briefly, a system is described for managing QC requirements. Generally, QC requirements are created and managed using a QC menu. QC Rules are created by selecting an assay panel (e.g. respiratory panel) and then selecting a target on that assay panel (e.g. Coronavirus, Influenza A, Influenza B, etc.). for each target, the user selects a run frequency and target rotation. The Days Until Due is automatically populated by a processor on the processing instrument or a processor remotely connected to the processing instrument.

### BRIEF DESCRIPTION OF THE DRAWINGS

FIG. 1 is a flow chart of an example process for creating, managing and viewing a QC Rule.

FIG. 2 is a flow chart of an example process for processing an external control.

FIG. 3 is a flow chart of an example process for processing a sample.

FIG. 4 is a flow chart of an example process for updating the Days Until Due.

FIG. 5 is a schematic diagram of an example of a system 500 to aid laboratories manage QC requirements.

FIG. 6 is a block diagram representing an exemplary software architecture and information flow for system 600.

FIG. 7 is a flow chart of an example process for creating, managing, deleting, viewing, and enabling/disabling Dynamic Panel Rules.

FIG. 8 is a flow chart of an example process for processing a sample according to Dynamic Panel Rules.

FIG. 9 is a schematic diagram of an example of a system 900 to aid laboratories manage Dynamic Panel Rules.

### DETAILED DESCRIPTION

### Definitions

As used in the claims and herein, the following terms have the following definitions:

Assay Analysis Module: An Assay Analysis Module contains the necessary information, including call logic, and target names to interpret raw data into a qualitative result. The AAM is contained inside the ADF and includes analysis parameters. “ADF” means Assay Definition File. An ADF is a single compressed file containing component files for running a software assay protocol and analyzing generated data. Sometimes, Protocol and ADF are used interchangeably. The Assay Definition File is composed of an OPUS file and an AAM file.

“Assay controls” means controls run in conjunction with the patient sample and indicate that the assay was run properly.

“Assay panel” or “panel” means is a predetermined group of medical tests used in the diagnosis of infection. For example, GenMark's ePlex system can run several different Blood Culture Identification (BCID) Panels. A panel has a plurality of targets.

“Assay targets” means the targets on each panel.

“Accession ID” means a unique alphanumeric character string that is used to unambiguously identify a particular record in a database.

“Base” means The computer, with software, that controls the towers, bays, and the display which are part of the diagnostic instrument.

“Bay” or “instrument bay” means a hardware module consisting of SBC, motors, sensors, and other electronics to receive and process a cartridge.

“Bay icon” or “Bay tile” means an icon on a GUI uniquely corresponding to a bay.

“Bay OQ Card” means a cartridge for performing quality control on a bay.

Cartridge or Consumable means a cartridge that includes the necessary components to perform a detection assay.

“CNS” means central nervous system.

“Days Until Due violation” means the Days Until Due are zero or less than 1, i.e., past due.

“Detection assay” means an assay including all of the reagents for carrying out a reaction for a specified target (e.g., microorganism nucleic acids, amplicons, etc.)

“Detection Result” means the result from processing a sample cartridge for a target. A Detection Result for a target can be “detected” or “not detected.”

“Detection Results” means the results from processing a sample cartridge for all of the targets on the cartridge.

“Diagnostic system” or “Diagnostic instrument” or “analysis device” is a manual, semi-automated or automated instrument that gathers information leading to the identification of a disease or disorder

“Diagnostic bay” or “bay” or “processing bay” or“sample processing bay” or “instrument bay” or “cartridge bay” is the part of the diagnostic instrument where a cartridge is inserted and processed. Bays as used herein are further described in U.S. patent application Ser. No. 14/062,860, U.S. Patent Publication No. 2015/0323555 and U.S. Pat. No. 9,598,722 which are herein incorporated by reference in their entireties.

“DNF” means Did Not Finish.

“DNS” means Does Not Start.

“Dynamic Report” a report containing Detection Results where targets have been selected/deselected for reporting in a Detection Report.

“Dynamic Target” is a target that is capable of being selected for inclusion or exclusion in a Detection Report. Dynamic targets are reported the hospital/doctor. Every assay on the cartridge is processed and only dynamic target results are reported to hospital/doctor. The dynamic target results represent a subset of the targets analyzed on the assay panel.

“Dynamic Target Rules” is a configurable rule which defines whether the Dynamic Target is reported in a Detection Report.

Dynamic Panel Ordering (DPO): The ability to create Dynamic Panel Rules.

“Dynamic Panel Rule” is a configurable rule which defines which Detection Results and/or targets are reported on a Detection Report. Dynamic Panel Rules may be configured either through LIS ordering or manual selection.

Dynamic Panel Name: The configurable name assigned to each Dynamic Panel Rule.

“Expected Targets” means the targets the assay panel is designed to detect.

“External control” means control material that is sampled and tested by the test system in much the same way a patient specimen is sampled and tested; therefore, it checks all components of the test system's analytic process.

“External Control Rule” or “QC Rule” means the rules established for the run frequency and/or rotation for the QC Control test.

GUI—A Graphical User Interface is a type of user interface that allows users to interact with electronic devices using images rather than text commands.

“Invalid Test” means a test which is invalid for a reason such as an internal control did not run properly.

“ISW” means the instrument software.

“LIS” means laboratory information system (LIS), a computer software that processes, stores and manages data from all stages of medical processes and tests.

“LIS hold” means that a result is not automatically sent to the hospital's LIS.

“LIS advanced rules” means an LIS rule that requires approval before the result is sent to the hospital's LIS.

LIS Panel Upload Code: The LIS Identifier in the instrument software specific to the panel. For panels using Dynamic Panel Ordering, multiple Dynamic Panel Upload codes will exist within a single panel (e.g., GI1, GI2, GI3).

“Opus” means the portion of the ADF and includes control parameters.

“OQ” means operational qualification.

“Patient cartridge” or “Sample Cartridge” or “PTO Cartridge” means a processing cartridge comprising a patient sample as opposed to a Quality Control Sample.

“POST” or A power-on self-test (POST) is a set of routines performed by firmware or software immediately after a computer is powered on, to determine if the hardware is working as expected.

“Processing instrument” means an instrument capable of processing a Sample Cartridge and generating a result.

“PTO” means pending test order. The physician test order is accepted by the Hospital LIS and a pending test order (PTO) is created once the patient sample is received and accessioned by the lab into the hospital LIS.

“Quality control” means a procedure or set of procedures intended to ensure that a manufactured product or performed service adheres to a defined set of quality criteria or meets the requirements of the client or customer.

“QC Accession ID” or “QC tag” means a unique alphanumeric character string that is used to unambiguously identify a particular QC Cartridge.

“QC Alert” means an alert given when a QC test is due but was missed.

“Quality control assay” or “Quality control test” means an assay/test comprising external controls.

“QC card” means a cartridge used for bay quality control.

“QC Cartridge” or “control cartridge” means a processing cartridge comprising a Quality Control Sample as opposed to a patient sample. The QC Cartridge is inserted into the testing process and treated as if they were a Sample Cartridge by being exposed to the same operating conditions.

“QC result” means the result of the QC test.

“QC result Alert” means an alert given when a QC test result is unexpected.

“QC Run” or “External Control Run” means processing a Quality Control Test.

“Quality Control Rule” means the parameters that define when a Quality Control Test should be run.

“Quality control samples” means special specimens inserted into the testing process and treated as if they were patient samples by being exposed to the same operating conditions. The purpose of including quality control samples in analytical runs is to evaluate the reliability of a method by assaying a stable material that resembles patient samples.

“Quality control test” or “Quality control assay” means an assay used to assess Quality Control as opposed to a patient sample.

“Quality control testing” is examining “control” materials of known substances to monitor the accuracy and precision of the processing instrument.

“QC menu” means a list of available QC tests.

“QC requirements” means the quality control requirements that must be satisfied to validate a processing instrument,

“Reserved bay” means a bay that is reserved.

“Run Frequency criterion” means how frequently the QC test will be run.

“Run Workflow” is the function of the instrument software where the user runs cartridges containing patient samples. Generally, the Run Workflow comprises loading a cartridge into the detection instrument, processing the cartridge and reporting the detection results.

“Regular Run” or “PTO run” means processing a cartridge containing a clinical sample.

“RP2” means a respiratory panel.

“Run view” or “Home screen” consists of the Menu bar on the top, the Body in the middle, and the Info bar on the bottom.

“Rotation” means the order of the Quality Control Tests for each assay on a panel.

“Sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. In contrast with some commercial systems that require some off chip handling of the sample, generally including sample extraction (cell lysis, for example), and sample preparation prior to detection. Thus, in accordance with aspects of the current system, a sample is loaded onto a BCID cartridge and the target analyte is extracted, amplified as necessary (for example, when the target analyte is a nucleic acid using polymerase chain reaction (PCR) techniques, although isothermal amplification methods can be utilized as well), and then detected using electrochemical detection, all on a microfluidic platform, generally referred to herein as a “multiplex cartridge” or a “fluid sample processing cartridge.” The BCID cartridge utilizes a sample preparation module as further described and shown in FIG. 15 of U.S. Pat. No. 9,598,722 (which is herein incorporated by reference in its entirety). In many embodiments, e.g. for the detection of human pathogens, the sample is a blood sample that is treated as outlined herein. Environmental samples include environmental material such as surface matter, soil, water, crystals and industrial samples. Such examples are not, however, to be construed as limiting the sample types applicable to the present technology.

“User selection” or “selecting” encompasses the diagnostic instrument receiving the user's selection.

“Viewing” by a user encompasses displaying by the diagnostic instrument.

As used herein, the term “about” means encompassing plus or minus 10%. For example, about 90% refers to a range encompassing between 81% and 99% nucleotides. As used herein, the term “about” is synonymous with the term approximately.

Unless otherwise indicated or the context suggests otherwise, as used herein, “a” or “an” means “at least one” or “one or more.”

The word “or” as used herein means any one member of a particular list and also includes any combination of members of that list.

### External Controls

External controls are run separate from a patient sample and indicate whether the system (diagnostic device) is running properly.

The expected assay targets define the external controls needed for each assay panel. Typically, there is a one to one correlation between the assay targets and the external controls needed for each assay panel. For example, if a respiratory panel can detect the following targets: Adenovirus, Coronavirus (229E, HKU1, NL63, OC43), Human Metapneumovirus, SARS-CoV-2, Human Rhinovirus/Enterovirus, Human Metapneumovirus, and Influenza A, then an external control is needed for each assay target.

Once the external controls are defined on the system, External Control Runs are performed. External Control Runs are performed on an order and frequency basis (as defined by the user, the hospital, CDC, etc.) or based on when the cartridge lot changes. The user can optionally elect to be alerted when external controls violate the run frequency and/or lot change criteria. An External Control Summary Report can be run to review all External Control Run results.

### Create New QC

When a laboratory receives a new diagnostic instrument or a new assay, they can create Quality Control Rules.

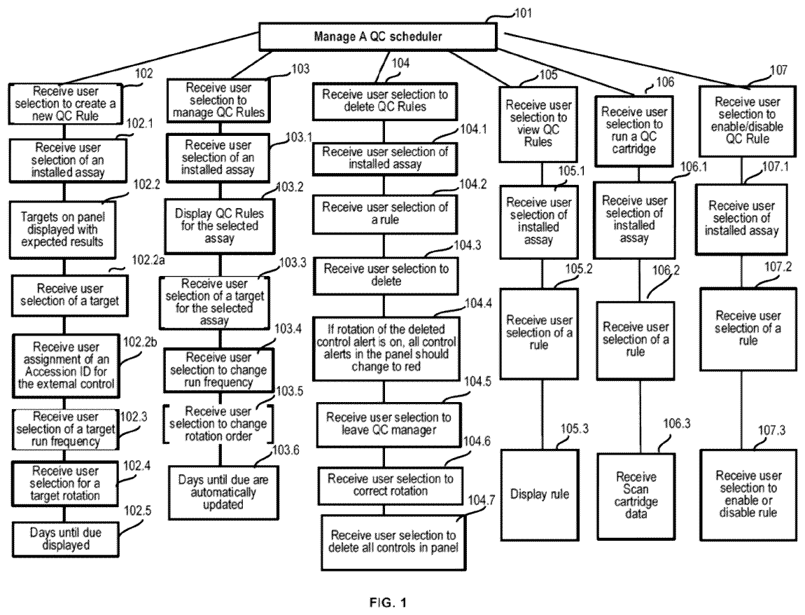

After navigating to the QC scheduler (FIG. 1 at step 101), the user selects between managing QC Rules (FIG. 1 at 103) or creating a new QC Rule (FIG. 1 at step 102). The QC scheduler is a drop-down menu in the GUI that allows a user to create or manage QC Rules. Selecting an item from a list of options (“Drop down menu”) is a common function of software user interfaces. As a user selects options from a dropdown menu, the subsequent items available for selection in the user interface changes.

In some embodiments, a user can create a new QC Rule by clicking on the Create a new QC Rule button (FIG. 1 at step 102). In some embodiments, a user can create a new QC Rule by clicking on the Manage QC Rules button (FIG. 1 at step 103). From there, the user clicks on an “edit” button for an assay panel (not shown in FIG. 1) to configure alerts specifically for that panel. In some embodiments, only those assays that have a QC Rule defined appear in the manage QC Rules screen. In some embodiments, all the assays that could have a QC Rule defined appear in the manage QC Rules screen whether or not a rule has been created already.

In some embodiments, selecting “create a new QC” (FIG. 1 at step 102) will produce a dropdown in the GUI which contains all of the installed assays on the system, aka the assay dropdown and also referred to as the “Create a New QC Assay Menu.” In some embodiments, selecting “create a new QC” (FIG. 1 at step 102) will produce an assay dropdown which contains assays for which a QC Rule can be set up on the system (FIG. 1 at step 102.1). In some embodiments, the assay dropdown contains only those assays installed on the system. For example, if the user only has a respiratory (RP) assay installed on the system, then only the RP assay is displayed in the assay dropdown. In some embodiments, only those assays installed on the system are preloaded into the assay dropdown.

In some embodiments, the assay dropdown (Create a New QC Assay Menu) contains all the assays that could be installed on the system. For example, if the user only has an RP assay installed on the system but could have RP and a gram-positive, gram-negative, or a fungal assay installed, all will be displayed. Even if a user has not purchased a blood culture panel, the gram-positive, gram-negative, and fungal assays will still appear in the Create a New QC Assay Menu.

In some cases, each assay has a unique colored tag, and the assay dropdown color tag matches the color tag of the assay.

In FIG. 1, at step 102.1, the user selects an installed assay (e.g., RP, gram-positive, gram-negative, or fungal), stated another way, the detection instrument receives a user selection.

Once the user selects an assay from the Create a New QC Assay Menu (102.1), the targets on the assay panel are displayed (102.2). In some embodiments, after selecting an assay from the Assay Dropdown, a Target Dropdown will be displayed in the GUI which contains all of the targets for the selected assay. In some embodiments, the Target Dropdown contains only those targets for the selected assay. For example, if the user has selected the respiratory (RP) assay, then only the RP targets are displayed in the Target Dropdown. In some embodiments, all of the targets for all of the assays installed on the system are displayed in the Target Dropdown. In some embodiments, the Target Dropdown displays as a grid each of the targets on the panel, the run frequency for each target, the rotation for each target and/or the Days Until Due for each target.

In some embodiments, the targets on the assay panel are further displayed in the Target Dropdown with the expected or predicted result. In some embodiments, each target's expected result is not detected, and each target will be marked “Not Detected”. In some embodiments, each target's expected result is “Detected” and each target will be marked “Detected.” In some embodiments, a first plurality of targets will be marked “Not Detected,” and a second different plurality of targets will be marked “Detected.” In some embodiments, the user creates a default expected result to be used for all QC operations, i.e., detected or not detected. In some embodiments, the user can generate a unique expected result for each QC Rule.

If the user wishes to include a customized expected result in lieu of the default expected result for one or more QC Rules/QC Classes, then the user selects a QC Rule/QC Class and enters the customized result for the QC Rule/QC Class. This step can be repeated if any additional customized expected results are to be included.

In some embodiments, the user selects a target (102.2a) such as for example, Adenovirus, Coronavirus (229E, HKU1, NL63, OC43), Human Metapneumovirus, SARS-CoV-2, Human Rhinovirus/Enterovirus, Influenza A, Human Rhinovirus/Enterovirus, Influenza A H1, Influenza A H1-2009, Influenza A H3, Influenza A H1-2009, Influenza B, Parainfluenza 1, Influenza B, Parainfluenza 2, Parainfluenza 3, Parainfluenza 4, Respiratory Syncytial Virus A, Respiratory Syncytial Virus B, Chlamydia pneumoniae, Mycoplasma pneumoniae, or Chlamydia pneumoniae (stated another way, the detection instrument receives a user selection). In some embodiments, the user does not need to select a target, they can proceed to the next step without making a selection, i.e., step 102.2a can be skipped.

In some embodiments, the user assigns an Accession ID for the external control (102.2b). In some embodiments, the user does not need to assign an Accession ID for the external control, they can proceed to the next step without making an assignment, i.e., step 102.2b can be skipped.

Once the user selects a target from the Target Dropdown (102.2), the target details are displayed. In some embodiments, after selecting a target from the Target Dropdown, a Target Detail Dropdown will be displayed in the GUI which contains all of the selected target's QC details such as expected/predicted result, Accession ID, Run Frequency, Rotation, Lot Alerts (on/off), Requires Valid Control (yes/no), Days Until Due, and combinations thereof for that target. The target QC details can be created/edited in the Target Detail Dropdown or in the Target Dropdown.

In some embodiments, the Target Detail Dropdown contains only target information for the selected target. In some embodiments, the Target Detail Dropdown contains only target information for the selected target that has been populated. For example, if the user has selected Parainfluenza 3 from the respiratory assay and only the expected result is known, then only the expected result for Parainfluenza 3 is displayed. In some embodiments, other details such as run frequency, rotation, Lot alert (on off), Requires Valid Control (yes/no), and Days Until Due are visualized in the Target Detail Dropdown but are grayed out or blank. Sometimes, other details such as run frequency, rotation, Lot Alerts (on/off), Requires Valid Control (yes/no), and Days Until Due are hidden until a user selects specific information. For example, in the Target Detail Dropdown the rotation is hidden until the user selects a run frequency. Once a run frequency is selected the option to select a rotation appears in the Target Detail Dropdown. After the user selects a rotation the Days Until Due appears in the Target Detail Dropdown and is populated.

In one embodiment, when a new QC Rule is entered by a user it is automatically added to the QC Rule database 505.

### Run Frequency

In the Target Detail Dropdown and/or the Target Dropdown, the user selects a run frequency for each selected target (FIG. 1 at step 102.3) (stated another way, the detection instrument receives a user selection).

In some embodiments, the system automatically assigns a run frequency to the targets on the panel, i.e., step 102.3 in FIG. 1 is skipped.

Under run frequency, the user selects the desired frequency between each control run (Weekly, Monthly, or Custom), In some embodiments, to establish a custom run frequency, the user selects a frequency which may be every X number of days, where X=1 to 365 days. In some embodiments, to establish a custom run frequency, the user selects a frequency which may be every X number of days, where X=1 to 100 days. In some embodiments, to establish a custom run frequency, the user selects a frequency, which may be monthly. In some embodiments, to establish a custom run frequency, the user selects a frequency, which may be weekly. In some embodiments, to establish a custom run frequency the user selects a frequency which may be every the first, second, third, fourth or fifth Monday, Tuesday, Wednesday, Thursday, Friday, Saturday or Sunday of the selected month(s).

In some embodiments, all of the targets on the assay have the same run frequency. In some embodiments, all of the targets on the assay have the same run frequency, and a single frequency is selected and applied in the Assay Dropdown prior to selecting a target (102.2a). In some embodiments, all of the targets on the assay have the same run frequency, and a run frequency is selected and applied to each target in the Target Detail Dropdown after selecting a target. In some embodiments, all of the targets on the assay have a different run frequency. In some embodiments, all of the targets on the assay have a different run frequency and the run frequency is selected for each target. In some embodiments, a first plurality of targets have a first run frequency and a second different plurality of targets have a second run frequency. In some embodiments, the first run frequency and second run frequency are different.

In some embodiments, step 102.3 (select a target run frequency) occurs before step 102.4 (select a target rotation). In some embodiments, the targets for an assay panel are assigned a target rotation. In some embodiments, the targets for an assay panel are not assigned a target rotation. In some embodiments, step 102.3 (select a target run frequency) occurs before step 102.4 (select a target rotation) and the user cannot advance to step 102.4 (select a target rotation) until after a target run frequency (102.3) is selected for each target. In some embodiments, after the user selects a run frequency for each target, a run rotation option becomes available. In some embodiments, step 102.3 (select a target run frequency) appears in the Target Detail Dropdown before step 102.4 (select a target rotation) but the user can advance to step 102.4 (select a target rotation) before a target run frequency (102.3) is selected for each target.

In some embodiments, step 102.4 (select a target rotation) occurs before step 102.3 (select a target run frequency) and the user cannot advance to step 102.3 (select a target run frequency) until after a target rotation (102.4) is selected for each target. In some embodiments, after the user selects a run rotation for each target, a run frequency option becomes available (i.e., appears or is no longer grayed out) in the Target Detail Dropdown.

### Rotation

In some embodiments, the system automatically assigns a rotation order to the targets on the panel, i.e., step 102.4 in FIG. 1 is skipped.

In some embodiments, in the Target Detail Dropdown and/or the Target Dropdown, the user selects a rotation for each selected target (FIG. 1 at step 102.4) (stated another way, the detection instrument receives a user selection). In some embodiments, the user can turn on or off a rotation. When a user turns off a rotation for an assay panel, the assay panel target external controls are assigned default orders. When a user turns on a rotation for an assay panel, the assay panel target external controls are not assigned default orders. The rotation of the assay panel target external controls can be defined in the Target Detail Dropdown and/or the Target Dropdown.

To assign the assay panel target external controls a rotation order the user inserts a rotation order into the rotation order selection.

In some embodiments, to assign the assay panel target external control rotation order, the user manually enters a rotation order into the rotation order selection box, i.e., first, second, third, etc. In some embodiments, by default, the rotation order selection boxes are empty, and the user fills them in. In some embodiments, by default, the rotation order selection boxes are filled in, and the user only changes the rotation order if desired. In some embodiments, to assign the assay panel target external control rotation order, the user selects from the available rotation orders in a rotation order dropdown wherein the number of available rotation orders matches the number of targets on the panel.

In some embodiments, if the user doesn't assign a valid rotation order for some controls, the “Days Until Due” parameter for them is not listed and frequency for those controls is not enforced. In some embodiments, the user cannot save a modification if a logical order has not been met. In some embodiments, a user cannot save controls if rotation is ON and an order is not assigned to all controls.

### Rotation Alert

In some embodiments, when a run frequency is defined a rotation alert can optionally be turned on. In some embodiments, when a rotation order is defined a rotation alert can optionally be turned on. In some embodiments, when a run frequency and rotation order are defined a rotation alert can optionally be turned on. The rotation alert is an alert that is sent when the rotation order is violated.

### Days Until Due

In some embodiments, after the assay panel target external control has been assigned a run frequency and rotation order (either by the user or by default), the Days Until Due is automatically displayed. In some embodiments, the Days Until Due will not be updated until after both the run frequency and rotation order are assigned to a target.

In some embodiments, a notification is sent when the Days until Due is less than 30, less than 25, less than 20, less than 10, less than 5, less than 1, or 0 days. In some embodiments, a notification is sent when the Days until Due is between 0 and 30, 0 and 25, 0 and 20, 0 and 10, 0 and 5, 0 and 1, or 0 days.

In some embodiments, the user selects the “Apply” button when they are done configuring the QC Rule(s) for the assay panel. In some embodiments, the new QC Rule is automatically saved. In some embodiments, the “Apply” or “Save” button is grayed out until a minimum amount of information is added to the rule. In some embodiments, the minimum information is the target run frequency. In some embodiments, the minimum information is the target rotation. In some embodiments, the minimum information is the target run frequency and target rotation.

### Enabled/Disabled Assay Targets

In some embodiments, the user can enabled/disable some or all targets from the assay. After navigating to the QC scheduler (FIG. 1 at step 101), the user selects between managing QC Rules (FIG. 1 at 103) or creating a new QC Rule (FIG. 1 at step 102). After selecting manage a QC rule or create a new QC rule, the assays are displayed. After the user selects an assay, the targets are displayed. The user can select or deselect different targets on the panel. Deselecting a target on a panel will remove the target from QC testing (frequency and rotation), even if the target has an associated QC Rule. Deselecting a target on a panel will automatically update the Days Until Due for the other targets on the panel according to their frequency and rotation in view of the deselected target.

Creating QC Rules can be further understood by the following numbered paragraphs:

Paragraph 1. In some embodiments, a method for creating a Quality Control Rule on a detection instrument is provided. The method includes receiving by the detection instrument a user selection of a first installed assay; receiving by the detection instrument the user selection of a target from the first installed assay; receiving by the detection instrument the user selection of a run frequency for the first target; generating by the detection instrument a first QC test; and processing by the detection instrument the first QC test according to the run frequency.

Paragraph 2. In some embodiments, the method for creating a Quality Control Rule on a detection instrument of claim 1 further includes receiving by the detection instrument the user selection of a second target from the first installed assay; receiving by the detection instrument the user selection of a run frequency for the second target; receiving by the detection instrument the user selection of a QC test rotation order for the first target and second target; and processing by the detection instrument the first QC test according to the run frequency and test rotation order.

Paragraph 3. In some embodiments, the method for creating a Quality Control Rule on a detection instrument of any preceding paragraph includes where the Days Until Due for the first QC test are automatically displayed by the detection instrument when the run frequency and/or test rotation order for the first QC test is generated.

Paragraph 4. In some embodiments, the method for creating a Quality Control Rule on a detection instrument of any preceding paragraph includes running the first QC test includes obtaining a first QC test result wherein if the first QC test result does not match a first QC expected test result the detection instrument sends an alert.

Paragraph 5. In some embodiments, the method for creating a Quality Control Rule on a detection instrument of any preceding paragraph further includes receiving by the detection instrument the user selection of a second installed assay; receiving by the detection instrument the user selection of a third target from the second installed assay; receiving by the detection instrument the user selection of a run frequency for the third target, thereby generating a second QC test; and processing by the detection instrument the second QC test according to the run frequency.

Paragraph 6. In some embodiments, the method for creating a Quality Control Rule on a detection instrument of any preceding paragraph includes processing the first QC test comprises obtaining a first QC test result, processing the second QC test comprises obtaining a second QC test result, and generating by the detection instrument a report comprising the first and second QC test results.

Paragraph 7. In some embodiments, a method for creating a Quality Control Rule on a detection instrument is provided. The method includes receiving by the detection instrument a user selection of a first installed assay from a QC menu; receiving by the detection instrument a user instructions deselecting a first target from the first installed assay by the user; receiving by the detection instrument the user selection of selecting a second target from the first installed assay by the user; receiving by the detection instrument the user selection of a run frequency for the first target and second target by the user; and processing by the detection instrument the second QC test according to the run frequency and ignoring the first QC test's run frequency.

Paragraph 8. In some embodiments, a method for creating a Quality Control Rule on a detection instrument is provided. The method includes providing by the detection instrument a QC scheduler drop-down wherein there are no installed assays; displaying by the detection instrument “there are no installed assays”; and preventing by the detection instrument the user from creating a QC rule.

Paragraph 9. In some embodiments, a method for creating a Quality Control Rule on a detection instrument is provided. The method includes providing by the detection instrument a QC scheduler drop-down wherein there is at least one installed assay and not QC Rules; displaying by the detection instrument “there are no QC Rules”; and displaying by the detection instrument a “create a new QC button.”

Paragraph 10. In some embodiments, a method for creating a Quality Control Rule on a detection instrument is provided. The method includes providing by the detection instrument a QC scheduler drop-down wherein there is at least one installed assay and at least one installed QC Rule for the assay; displaying by the detection instrument an assay drop down for assays having a QC Rule; receiving by the detection instrument a user selection of an assay having a QC Rule; displaying by the detection instrument an assay drop down comprising the QC Rules for the selected assay; and displaying by the detection instrument a “create a new QC button” and a “manage a QC button.”

Paragraph 11. The method for creating a Quality Control Rule on a detection instrument of any preceding paragraph, further comprising receiving by the detection instrument a user defined QC Accession ID for each QC Rule created.

Paragraph 12. The method for creating a Quality Control Rule on a detection instrument of any preceding paragraph, further comprising receiving by the detection instrument a user defined comment for one or more QC Rule created.

Paragraph 12.1. The method for creating a Quality Control Rule on a detection instrument of any preceding paragraph, further comprising updating by the detection instrument the QC Scheduler to reflect the frequency, rotation order, Lot Alert (on/off), Require Valid Control (yes/no) and/or Days Until Due parameters.

Paragraph 13. The method for creating a Quality Control Rule on a detection instrument of paragraph 10, wherein displaying by the detection instrument an assay drop down comprising the QC Rules further comprising displaying the expected result for each assay target.

Paragraph 14. A method for creating a Quality Control Rule on a detection instrument, the method comprising: automatically populating, by the detection instrument, a QC Rule for an installed assay.

Paragraph 15. A diagnostic instrument based Quality support system for strengthening a healthcare provider's quality program comprising: a diagnostic instrument having a processor, memory, and access to computer readable media; a computer program stored on computer readable media having a quality assurance module; and a user interface displayed on a monitor for interaction with said computer program.

Paragraph 16. The diagnostic instrument based Quality support system of paragraph 15, further comprising a network connection between the diagnostic instrument and a computer network for allowing remote access to the computer program;

Paragraph 17. The diagnostic instrument based Quality support system of an preceding paragraph, a pixel mapped calendar stored on computer readable media and graphically displayed on a monitor having a plurality of colored geometries arranged in rows and columns for each date, each colored geometry representing status of a quality assurance task and being assigned a color corresponding to a current status of a quality assurance task;

Paragraph 18. The diagnostic instrument based Quality support system of an preceding paragraph, a reminder function stored on computer readable media and displayed on a monitor containing a visual representation of task compliance for each quality assurance task.

### Manage QC Rules

After a QC Rule has been created (Step 102 in FIG. 1), it can be managed (Step 103 in FIG. 1).

After navigating to the QC scheduler (FIG. 1 at step 101), the user selects between creating a new QC Rule (FIG. 1 at step 102) or managing QC Rules (FIG. 1 at 103) from the QC scheduler dropdown.

In some embodiments, selecting “Manage QC Rules” (FIG. 1 at 103) will produce a Manage QC Rules Dropdown which displays assays with QC Rules set up on the system. This is the “Manage QC Rules Menu.” The Manage QC Rules Menu reflects the most recent control parameters (expected results, run frequency, target rotation). In some embodiments, each installed assay has a corresponding colored assay tag and the same color is used in the GUI when the assay name is displayed.

In some embodiments, selecting “Manage QC Rules” (FIG. 1 at step 103) will produce an Installed Assay Manage QC Rules Dropdown which contains the installed assays on the system (e.g., RP2, gram-positive, gram-negative, or fungal). The user selects the installed assay (FIG. 1 at 103.1) (stated another way, the detection instrument receives a user selection) and then a second drop-down menu appears which contains the QC Rules that have been set up on the system for that assay (the Manage QC Rules Menu) and the user selects the QC Rule (FIG. 1 at 103.2). In some embodiments, if there is only one QC Rule set up on the system, step 103.1 is skipped. Step 103.1 can also be skipped if, as in some embodiments, selecting “Manage QC Rules” (FIG. 1 at step 103) produces an assay dropdown which contains all of the QC Rules that have been set up on the system, i.e., step 103.2 displays all of the control alerts set up on the system.

Once the user selects a target (FIG. 1 at step 103.3) (stated another way, once the detection instrument receives a user target selection) the user can change the run frequency (FIG. 1 at step 103.4), rotation order (FIG. 1 at step 103.5) or both. When the run frequency (FIG. 1 at step 103.4) and/or rotation order (FIG. 1 at step 103.5) is changed, the Days Until Due are automatically updated (FIG. 1 at step 103.6).

As with creating a rule, when editing a rule, the user selects the desired frequency between each control run (Weekly, Monthly, or Custom), In some embodiments, to change a custom run frequency, the user selects a frequency which may be every X number of days, where X=1 to 365 days. In some embodiments, to change a custom run frequency, the user selects a frequency which may be every X number of days, where X=1 to 100 days. In some embodiments, to change a custom run frequency, the user selects a frequency which may be monthly. In some embodiments, to change a custom run frequency, the user selects a frequency which may be weekly. In some embodiments, to change a custom run frequency, the user selects a frequency which may be every first, second, third, fourth, or fifth Monday, Tuesday, Wednesday, Thursday, Friday, Saturday, or Sunday. In some embodiments, to change a custom run frequency the user selects a frequency which may be a particular day of the selected month(s), e.g. the second Tuesday of every month or the first Monday of every third month.

As with creating a rule, when editing a rule, the user selects the desired run rotation. When a user turns off a rotation for an assay panel, the assay panel target external controls are assigned default orders. When a user turns on a rotation for an assay panel, the assay panel target external controls are not assigned default orders. To change the assay panel target external control's rotation order, the user changes the rotation order in the rotation order selection box. To change the assay target external control rotation order, the user manually enters a different rotation order in the rotation order selection box, i.e., first, second, third, etc. To change the assay target external control rotation order, the user selects from the available rotation orders from a rotation order dropdown wherein the number of available rotation orders matches the number of targets on the panel.

In some embodiments, (when creating a QC Rule or managing a QC Rule) if all of the assay target external controls do not have a rotation order, an alarm will sound or an alert will be given. In some embodiments (when creating a QC Rule or managing a QC rule), if any assay target external controls rotation order are duplicated (e.g., two external controls are assigned to go second), an alarm will sound or an alert will be given. In some embodiments, it is not possible to give two assay target external controls duplicate rotations (e.g., two assays are assigned to go second) because the user must select from a drop-down of available frequencies and once one is selected, it cannot be selected again.

In some embodiments, the user selects the “Apply” button when they are done editing the alerts for the assay panel. In some embodiments, the edited QC Rule is automatically saved. In some embodiments, when a run frequency or target rotation is updated, the user selects the “Reset Days Until Due” button, which then updates the Days Until Due.

In some embodiments, the Days Until Due value is based on the run frequency. In some embodiments, the Days Until Due value is based on the rotation order. In some embodiments, the Days Until Due value is based on the run frequency and rotation order.

In some embodiments, not every QC parameter can be changed. For example, in some embodiments, if an external control has not been run, a user may only edit the QC Accession ID, Comments, and Expected Targets. In some embodiments, if the external control has been run, a user may only edit the Comments and Expected Targets, they can't edit the QC Accession ID or Panel.

In some embodiments, when the manage QC Rule is selected from the QC scheduler dropdown, the user can perform one or more of the following: view all QC Rules, print QC barcode, run QC, view run history (a report of the QC tests performed for that assay or panel), edit QC Control, reset due date, duplicate QC Rule, and/or delete a QC Rule.

### Lot Alerts

In some embodiments, when a new assay is created or edited, the user can turn on a Lot Alert.

In some embodiments, the Lot Alert is on for each QC Rule. In some embodiments, the Lot Alert is off for each QC Rule. In some embodiments, the Lot Alert is on for a first plurality of QC Rules and is off for a second plurality of QC Rules.

A user may manually turn on or off the Lot Alert when creating or managing a QC Rule.

When a Lot Alert is on, the system sends an alert (warning sound, email, text message, instant message, etc.) that notifies the user that the Sample Cartridge lot is changed and a new QC Cartridge must be processed before the Sample Cartridge can be processed.

In some embodiments, the Lot Alert is activated when a user scans a Sample Cartridge and the lot information on the Sample Cartridge does not match the lot information in the last QC Cartridge run.

In some embodiments, Lot Alert is on for a first assay panel and off for a second assay panel wherein the first assay panel and second assay panel are different.

In some embodiments, the Lot Alert is on for a first assay target and off for a second assay target on the same assay panel wherein the first assay target and second assay target are different.

### “Require Valid Control to Perform Diagnostic Tests” Option

In some embodiments, when a new assay is created or edited, the user can turn on a requirement that a valid control be run prior to processing a Sample Cartridge. In some embodiments, when a new assay is created or edited, the user can turn on a Lot Alert and require that a valid control be run prior to processing a Sample Cartridge and after the cartridge lot has changed.

In some embodiments, the Require Valid Control to Perform Tests Option is on for each QC Rule. In some embodiments, the Require Valid Control to Perform Tests Option is off for each QC Rule. In some embodiments, the Require Valid Control to Perform Tests Option is on for a first plurality of QC Rules and is off for a second plurality of QC Rules.

When a Require Valid Control to Perform Tests Option is on, the system sends an alert (warning sound, email, text message, instant message, etc.) that notifies the user that the QC Cartridge has not been processed for the particular Sample Cartridge when that Sample Cartridge is scanned or inserted into the instrument. In some embodiments, if the Sample Cartridge is for a first panel and a first panel QC Rule is outstanding, the system will not process the first panel Sample Cartridge. For example, if the Sample Cartridge is a Respiratory cartridge and a Respiratory QC Rule is outstanding, the system will not process the Respiratory Sample Cartridge.

In some embodiments, if the Sample Cartridge is for a first panel and a second panel QC Rule is outstanding, the system will process the first panel Sample Cartridge. For example, if the Sample Cartridge is a Respiratory cartridge and a Blood culture QC Rule is outstanding, the system will process the Respiratory Sample Cartridge.

In some embodiments, the Require Valid Control to Perform Tests Option is activated when a user scans a Sample Cartridge and the cartridge information on the Sample Cartridge is compared to the QC Rules and if a QC Rule related to that Sample Cartridge is violated, then the system will not process the Sample Cartridge.

In some embodiments, the Require Valid Control to Perform Tests Option is on for a first assay panel and off for a second assay panel wherein the first assay panel and second assay panel are different.

In some embodiments, the Require Valid Control to Perform Tests Option is on for a first assay target and off for a second assay target on the same assay panel wherein the first assay target and second assay target are different.

### Editing Control Alerts

In some embodiments, the external control alert can be edited. For example, in some embodiments, the alert (rotation alert, expected result alert or lot alert) can be modified for a particular panel or target. For example, if a particular target requires a Lot Alert but the rest of the targets in the panel do not require a Lot Alert, then the Lot Alert can be modified for the single target on the panel. As another example, if a particular target requires a Rotation Alert (i.e., the target was missed in the rotation) but the rest of the targets in the panel do not require a rotation alert, then the rotation alert can be modified for the single target on the panel. Again, if a particular target requires that the Valid Control Option be on (i.e., the target's control must be run before a sample can be processed) but the rest of the targets in the panel do not require that the Valid Control Option be on, then the Valid Control Option can be modified for the single target on the panel.

Editing QC Control Alerts can be further understood by the following numbered paragraphs:

Paragraph 1. A method for managing quality control alerts on a detection instrument, the method comprising:
displaying a detection instrument a control alert for a first QC Rule; receiving by the detection instrument a user modification to the first QC Rule; processing by the instrument the first QC test according to the modifications.

Paragraph 2. The method for managing quality control alerts on a detection instrument of paragraph 1, wherein modifications comprises turning the run frequency Off, turning the rotation Off, changing the run frequency to a different value, changing the rotation to a different value.

Paragraph 3. The method for managing quality control alerts on a detection instrument of paragraph 1, further comprising updating the Days Until Due for the first QC Rule based on the modification.

Paragraph 4. The method for managing quality control alerts on a detection instrument of paragraph 1, further comprising updating the Days Until Due for a second QC Rule based on the modification to the first QC Rule.

### Editing a QC Rule as a Supervisor

In some embodiments, different external control features can only be changed by someone with the proper credentials, such as a supervisor or administrator. In FIG. 1, there is a step before step 103.1 (select an installed assay) or before step 103.3 (select a target), or before step 103.4 (change run frequency), or before step 103.5 (change rotation order). In these embodiments, the system will ask the supervisor to enter a passcode or scan a badge before selecting an installed assay, selecting a target, changing a run frequency, or changing a rotation order. After the supervisor has entered the passcode or swiped a badge, they can select an installed assay and proceed through the steps to manage a QC Rule. In some embodiments, if the supervisor does not enter a passcode or swipe a badge, the QC rule cannot be changed.

In some embodiments, supervisor/administrator approval is needed to change alerts (turn them on or off), such as Lot Alert, Rotation Alert, and/or Expected Results Alert. In some embodiments, supervisor approval is needed to change the Requires Valid Control Option (turn on or off).

In some embodiments, if the control has not been run, a Supervisor user may edit the QC Accession ID, add a comment to the QC Rule, and define the predicted result for the targets (detected or not detected). In some embodiments, the supervisor cannot edit the targets on a Panel (add or remove). In some embodiments, the supervisor can edit the targets on a Panel (add or remove).

In some embodiments, if the control has been run, a Supervisor user may only edit a comment and predicted result for a target. In some embodiments, if the control has been run, a supervisor cannot edit the QC Accession ID or targets on a Panel

Managing QC Rules can be further understood by the following numbered paragraphs:

Paragraph 1. A method for managing quality control rules on a detection instrument, the method comprising:
receiving by the detection instrument a user selection of a first QC test from a QC menu; receiving by the detection instrument user modification instructions for the run frequency for the first QC test; and processing by the instrument the first QC test according to the run frequency.

Paragraph 2. The method for managing quality control rules on a detection instrument of claim 1, further comprising: receiving by the detection instrument the user selection of a second QC test from the QC menu; receiving by the detection instrument user modification instructions for the run frequency for the second QC test; and receiving by the detection instrument user modification instructions for the QC test rotation order for the first QC test and second QC test.

Paragraph 3. The method for managing quality rules on a detection instrument of any preceding paragraph, wherein the Days Until Due for the first QC test and second QC test are automatically displayed by the detection instrument when the run frequency is modified.

Paragraph 4. The method for managing quality rules on a detection instrument of any preceding paragraph, further comprising running the QC test wherein running the first QC test comprises obtaining a first QC test result wherein if the first QC test result does not match a first QC expected test result the detection instrument sends an alert.

Paragraph 5. The method for managing quality rules on a detection instrument of paragraphs 2-4, further comprising running the QC test wherein running the first QC test comprises obtaining a first QC test result and running the second QC test comprises obtaining a second QC test result and further comprising generating a report comprising the first and second QC test results.

Paragraph 6. The method for managing quality rules on a detection instrument of paragraphs 2-4, further comprising updating by the detection instrument the QC Scheduler to reflect the updated frequency, rotation order, Lot Alert (on/off), Require Valid Control (yes/no) and/or Days Until Due parameters.

### Delete an External Control Rule

In some embodiments, an External Control Rule can be deleted. This can be accomplished at the individual control level, or as part of a bulk action.

### Delete an Individual External Control Rule

After navigating to the QC scheduler (FIG. 1 at step 101), the user selects between creating a new QC Rule (FIG. 1 at step 102), managing QC Rules (FIG. 1 at 103) or deleting QC Rules (FIG. 1 at 104) from the QC scheduler dropdown.

From the manage QC Rules (FIG. 1 at step 101) drop-down, the user selects Delete QC Rule (FIG. 1 at step 104) to delete an External Control Rule. In some embodiments, selecting “Delete QC Rule” (FIG. 1 at step 104) will produce an assay dropdown which contains a list of all the installed assays that have a QC Rule. (FIG. 1 at step 104.1). The user selects the QC Rule (FIG. 1 at step 104.2) and deletes it (FIG. 1 at step 104.3).

### To Delete Multiple External Control Rules:

From the manage QC Rules (FIG. 1 at step 101) drop-down, the user selects Delete QC Rule (FIG. 1 at step 104) to delete all of the External Control Rules associated with an assay panel.

In some embodiments, the user selects the assay (FIG. 1 at step 104.1) and deletes it (FIG. 1 at step 104.3). Stated another way, the detection instrument receives a user assay selection (FIG. 1 at step 104.1) and instructions to delete it (FIG. 1 at step 104.3).

If a QC Rule is deleted and Rotation (meaning the deleted QC rule is in a QC rotation) for that panel is on (FIG. 1 at step 104.4), and the panel no longer has a logical QC Rule order, all QC Rules in that panel change to red text. If a user attempts to navigate away from the QC tab without fixing the logical rotation, a dialogue appears asking them to “QC Alerts for the highlighted controls have been disabled. Please use Manage QC Rules to correct the rotation order or delete all controls for the affected panel to proceed.” The user may leave the QC manager (FIG. 1 at step 104.5), correct the rotation (FIG. 1 at step 104.6) or delete the remaining QC Rules in the panel (FIG. 1 at step 104.7).

Deleting QC Rules can be further understood by the following numbered paragraphs:

Paragraph 1. A method for deleting a Quality Control Rule on a detection instrument, the method comprising:
selecting from a QC menu a first installed assay; selecting a first target from the first installed assay;
selecting a first QC test for the first target; and deleting the first QC test from the QC menu.

Paragraph 2. A method for deleting a Quality Control Rule on a detection instrument, the method comprising:
selecting from a QC menu a first installed assay; selecting a first QC Rule from the first installed assay; and
deleting the first QC Rule from the QC menu.

Paragraph 3. The method for deleting a Quality Control Rule on a detection instrument, the method comprising: selecting from a QC menu a first installed assay; and deleting the first installed assay from the QC menu.

Paragraph 4. The method for deleting a Quality Control Rule on a detection instrument of any preceding paragraph, wherein the Days Until Due for a second QC Rule in the QC menu is automatically updated when the first QC test, first QC Rule or first installed assay is deleted from the QC menu.

Paragraph 5. A method for deleting a Quality Control Rule on a detection instrument, the method comprising:
receiving by the detection instrument a user selection of a first installed assay from a QC menu; receiving by the detection instrument the user selection of a first target from the first installed assay; receiving by the detection instrument the user selection of a first QC test for the first target; receiving by the detection instrument the user instructions deleting the first QC test from the QC menu; and deleting by the detection instrument the first QC test from the QC menu.

### View an External Control Schedule

In some embodiments, external control schedules can be viewed. After navigating to the QC scheduler (FIG. 1 at step 101), the user selects between managing QC Rules (FIG. 1 at 103) creating a new QC (FIG. 1 at step 102), deleting a QC Rule (FIG. 1 at 104) or viewing an external control schedule (FIG. 1 at 105).

### Viewing an Individual External Control Rule

From the Manage QC Scheduler (FIG. 1 at step 101) drop-down, the user selects View QC Schedule (FIG. 1 at step 105). In some embodiments, selecting “View QC Rule” (FIG. 1 at step 105) will produce an assay dropdown which contains a list of all the installed assays that have a QC Rule. (FIG. 1 at step 105.1). The user selects the assay from the drop-down which then displays a drop-down of the QC Rules for that assay. The user can select a QC Rule (FIG. 1 at step 105.2) from the drop-down and views it (FIG. 1 at step 105.3). Stated another way, the detection instrument receives a user assay selection (FIG. 1 at step 105.1), receives a user rule selection (FIG. 1 at step 105.2) and displays the selected rule (FIG. 1 at step 105.3).

### Viewing Multiple External Control Rules

Select the assay (FIG. 1 at step 105.1) and view all the QC rules associated with it (FIG. 1 not shown).

In some embodiments, the Days Until Due are viewed. In some embodiments, there is a display showing when the QC Rule was last run. In some embodiments, there is a display showing when the QC Rule was last successfully run. QC Rule information can include creator username, QC rule creation date, timestamp the last time the QC Rule was run, bay serial number for the last time the QC Rule was run, bay location for the last time the QC Rule was run, QC Rule violations, QC Cartridge serial number (if any for last time the rule was run), QC Cartridge Accession ID (if any for the last time the rule was run).

Viewing QC Rules can be further understood by the following numbered paragraphs:
Paragraph 1. A method for viewing a Quality Control Rule on a detection instrument, the method comprising:
selecting from a QC menu a first installed assay; selecting a first target from the first installed assay; selecting a first QC test for the first target; and viewing the first QC test from the QC menu.

Paragraph 2. The method for viewing a Quality Control Rule on a detection instrument, the method comprising: selecting from a QC menu a first installed assay; and viewing the first installed assay rules from the QC menu.

Paragraph 3. A method for viewing a Quality Control Rule on a detection instrument, the method comprising:
receiving by the detection instrument a user selection of a first installed assay from a QC menu; receiving by the detection instrument the user selection of a first target from the first installed assay; receiving by the detection instrument the user selection of a first QC test for the first target; and displaying by the detection instrument the first QC test from the QC menu.

### Enabling/Disabling an External Control

After navigating to the QC scheduler (FIG. 1 at step 101), the user selects between creating a new QC Rule (FIG. 1 at step 102), managing QC Rules (FIG. 1 at 103) deleting QC Rules (FIG. 1 at 104), viewing QC Rules (FIG. 1 at 105) or enabling/disabling QC Rules (FIG. 1 at 107) from the QC scheduler dropdown.

From the manage QC Rules (FIG. 1 at step 101) drop-down, the user selects enable/disable QC Rule (FIG. 1 at step 107) to enable/disable an External Control Rule. In some embodiments, selecting “Enable/Disable QC Rule” (FIG. 1 at step 107) will produce an assay dropdown, which contains a list of all the installed assays that have a QC Rule. (FIG. 1 at step 107.1). The user selects the QC Rule (FIG. 1 at step 107.2) and enables/disabled it (FIG. 1 at step 107.3). In some embodiments, the user can select more than one QC Rule to enable or disable. In one embodiment, a first QC is enabled and a second QC is disabled. In one embodiment, a first QC is enabled and a second QC is enabled. Stated another way, the detection instrument receives a user assay selection (FIG. 1 at step 107.1), receives a user rule selection (FIG. 1 at step 107.2), and receives a user instructions to enable/disable (FIG. 1 at step 107.3).

In some embodiments, a QC Rule that has been disabled is still viewable in the GUI.

### Running an External Control

In some embodiments, to begin an External Control Run, the user navigates to the QC scheduler and selects “run an external control” from the drop down menu. After navigating to the QC scheduler (FIG. 1 at step 101), the user selects between managing QC Rules (FIG. 1 at 103) creating a new QC (FIG. 1 at step 102), deleting a QC Rule (FIG. 1 at 104), viewing an external control schedule (FIG. 1 at 105) and running an external control (FIG. 1 at 106).

In some embodiments, selecting “run a QC Cartridge” (FIG. 1 at step 106) will produce an assay dropdown, which contains a list of all the installed QC Rules. The user selects the QC Rule (FIG. 1 at step 106.2) from the drop-down and scans a QC Cartridge corresponding to the QC Rule (FIG. 1 at step 106.3). The system then prompts the user to scan a QC Cartridge for the selected external control.

In some embodiments, selecting “run a QC Cartridge” (FIG. 1 at step 106) will produce an assay dropdown, which contains a list of all the installed assays that have a QC Rule. The user selects an assay (FIG. 1 at step 106.1) which produces a drop-down of the QC rules associated with that assay. The user selects the QC Rule (FIG. 1 at step 106.2) from the drop-down and scans a QC Cartridge corresponding to the QC Rule (FIG. 1 at step 106.3). The system then prompts the user to scan a QC Cartridge for the selected external control. Stated another way, the detection instrument receives a user assay selection (FIG. 1 at step 106.1), receives a user rile selection (FIG. 1 at step 106.2) and receives scan cartridge data (FIG. 1 at step 106.3).

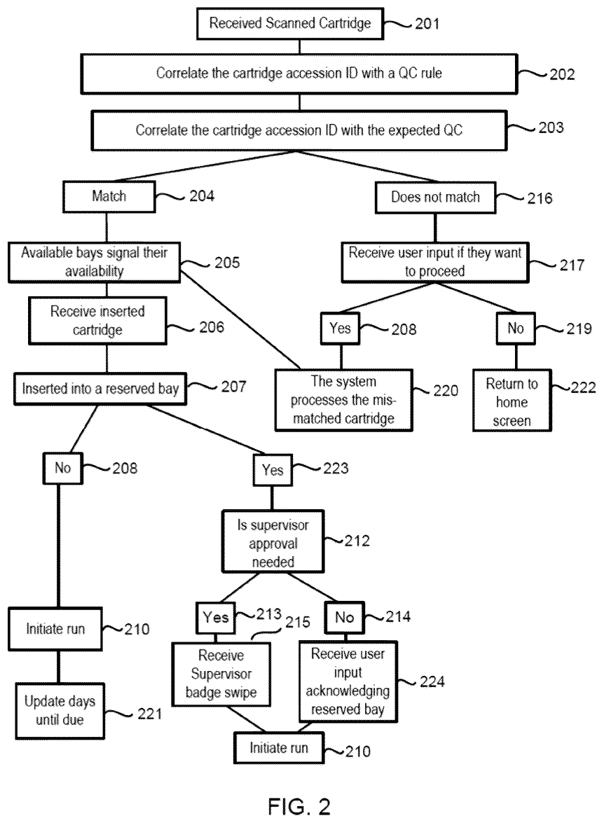

In some embodiments, to begin an External Control Run, the user scans a cartridge with a QC Control Accession ID (FIG. 2 at step 201). In some embodiments, the cartridge Accession ID is correlated with a QC run (FIG. 2 at step 202), i.e., the system recognizes that the cartridge is not a patient sample but is a QC Cartridge and it identifies the QC Rule applied to that QC Cartridge. In some embodiments, step 202 is skipped.

In some embodiments, the QC Cartridge Accession ID is correlated with the next expected QC run (FIG. 2 at step 203), i.e., the system recognizes that the cartridge is not a patient sample but is a QC Cartridge and it looks to see if the QC Cartridge is the next QC Cartridge it expects to run in the rotation.

In some embodiments, the cartridge Accession ID is correlated with a QC Control Accession ID, the assay, and/or the list of targets.

In one embodiment, when a QC Cartridge is in process the status of the QC result is “in process.”

### Mismatched QC Cartridge a.k.a Out of Order External Control Runs

In some embodiments, if the scanned QC barcode does not match the expected barcode of the QC that has the lowest Days Until Due (e.g. is due next) (FIG. 2 at step 216), the system alerts the user of the mismatch and the user is asked if they want to proceed (FIG. 2 at step 217). If the user selects yes, the system processes the mis-matched cartridge (FIG. 2 at step 220) the system proceeds to step 205 in FIG. 1. If the user selects no (FIG. 2 at step 219), the system will not process the mismatched cartridge and returns the user to the home screen (FIG. 2 at step 222). In some embodiments, if the user does not make a selection but loads a mismatched cartridge, the system alerts the user to the mismatch but processes the mismatched QC Cartridge. In some embodiments, if the user does not make a selection but loads a mismatched cartridge, the system alerts the user to the mismatch and will not process the mismatched QC Cartridge.

In some embodiments, if the scanned barcode does not match the expected barcode of the QC that has the lowest Days Until Due (e.g. is due next), the system alerts the user of the mismatch and requires them to select the proper control for the control that has the lowest Days Until Due.

### Matched QC Cartridge

### Running a QC Cartridge

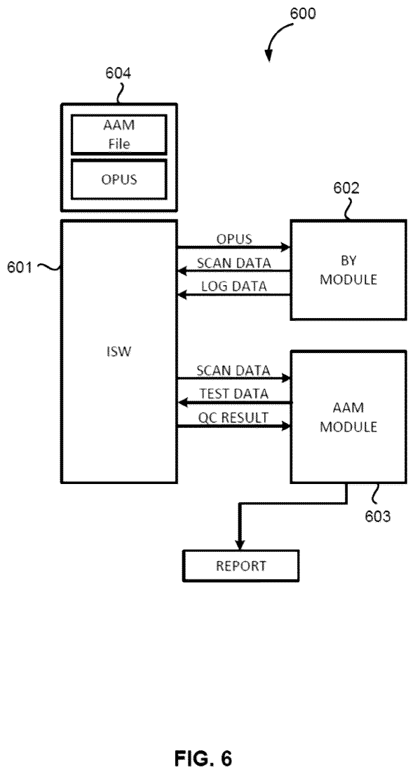

If a control cartridge is being processed (either because it is a match or the user has selected to proceed (FIG. 2 at step 220)), the LED light of all available bays changes to a designated color (such as white, or green) to indicate that they are ready to accept a control cartridge (FIG. 2 at step 205). In some embodiments, a display dialog prompts the user to insert a cartridge into any available bay and the timeout bar will count down (timeout is 30 seconds by default). If a cartridge is loaded into the instrument, the instrument initializes the control cartridge run (FIG. 2 at step 210). In some embodiments, if the QC Cartridge fails pre-flight processing an error is displayed on the GUI and the cartridge is ejected. In some embodiments, if the QC Cartridge fails pre-flight processing an error is displayed on the GUI, the cartridge is ejected and the Days Until Due is not updated. The QC Cartridge includes an AAM file (analysis parameters) and Opus file (processing instructions to generate sensor scan data). Generally, FIG. 6 is a block diagram representing an exemplary software architecture and information flow for system 600, according to some embodiments. FIG. 6 is a reproduction of FIG. 63 from U.S. Pat. No. 11,300,578 by Nguyen et al. but as it applies to a QC Cartridge as opposed to a Sample Cartridge. In particular this aspect of Nguyen et al. is incorporated by reference in its entirety. System 600 may comprise instrument software module (ISW) 601, bay software module 602, and an assay analysis module (AAM) 603. The ISW 601 may generally reside in an instrument (not shown) although portions of ISW 601 may also reside in client device (not shown) as further described in U.S. Pat. No. 10,935,561 which is also herein incorporated by reference in its entirety. The bay software module 602 resides in processing bay of an instrument (not shown) although portions or all of software module can also reside in other portions of instrument (not shown). The AAM module 603 can reside in one or more of instrument, client device, or server. To perform a test, an assay definition file (ADF) 604 may be received by the ISW 601. The ADF 604 may comprise two portions including an OPUS file and an AAM file. The OPUS file may include parameters that define all operations controlled by the execution of bay software module 602. The AAM file may include parameters that define analysis performed by AAM module 603. In operation, a QC Cartridge is loaded into an instrument bay. The bay module 602 may obtain scan data and log data which are sent to the ISW 601. The ISW sends the scan data to the AAM module 603. The AAM module 603 may generate a test result as a result of the AAM file parameters being applied to the scan data. The test result is reported to the ISW 601. The ISW 601 may generate a QC result. The QC result is reported to the AAM module 603. The AAM module sends a report to the GUI, hospital LIS or other end client indicating that the QC Cartridge was run and the expected result obtained.

### Running a QC Cartridge in a Reserved Bay

The analysis device can allow customers the ability to reserve a desired number of bays and require a password to be entered prior to using a reserved bay, with the intent of ensuring there is always a bay available for “STAT” tests or critical tests that enter the lab. The “reserved” bay(s) reserve the specified number of bays based on availability—not location. e.g. if 2 bays are reserved on a tower system with 6 bays, and 3 runs are currently being processed. A fourth sample or QC run can be inserted into any available bay. The remaining 2 bays will enter a reserved state, i.e., a sample or QC cartridge will not be processed unless the reservation is bypassed.

In some embodiments, the detection instrument is configured to reserve a specific bay location. This means, for example, bay number 6 is always reserved. In some embodiments, bays may be reserved for a specific assay. In some embodiments, bays may be reserved for a specific target. In some embodiments, bays may be reserved for CNS tests. CNS tests are at an increased risk of contamination, so reserving a bay for CNS tests reduces that contamination.

If the user inserts the cartridge into a reserved bay (FIG. 2 at step 207) and supervisor approval is required (FIG. 2 at step 212), a “supervisor sign-off” dialog box is displayed. A Supervisor will need to enter a unique code to process the control cartridge in a reserved bay or swipe a badge to give authorization. (FIG. 2 at steps 213 and 215). If a Supervisor does not to enter a unique code to process the control cartridge in a reserved bay or swipe a badge to give authorization the cartridge cannot be processed.

If the user inserts the cartridge into a reserved bay and supervisor approval is not required (FIG. 2 at step 214), an acknowledgement dialog box is displayed (not shown). The operator must click a button to acknowledge that a reserved bay is being used (FIG. 2 at step 224).

Reserving a bay for STAT testing can be further understood by the following numbered paragraphs:

Paragraph 1. A method managing a detection instrument comprising bays, the method comprising:
determining, by the detection device that a first bay should be reserved based on a reserve bay rule; reserving the first bay so that it will not process any sample cartridges; and processing a first sample cartridge in a second bay.

Paragraph 2. The method managing a detection instrument of Paragraph 1, further comprising: receiving, by the detection device a second sample cartridge; determining by the processing instrument that the second sample cartridge satisfies the reserve bay rule for the first bay; and processing the second sample cartridge in the first bay.

Paragraph 3. The method managing a detection instrument of Paragraph 1, wherein the reserve bay rule permits STAT orders to be processed and the second sample cartridge is a STAT order.

Paragraph 4. A method managing a detection instrument comprising bays, the method comprising:
determining, by the detection device that at least one bay should be reserved based on a reserve bay rule; determining that at least one bay is available for reservation; and processing a first sample cartridge on any available bay so long as there is at least one bay available for reservation.

Paragraph 5. The method of managing a detection instrument of Paragraph 4, further comprising:
identifying, by the detection device, a first available bay (i.e., not processing a cartridge); determining, by the detection device, that there are no more bays available for reservation; receiving a sample cartridge that does not satisfy the reserve bay rule; ejecting the cartridge.

### Bay Dialog Box:

In some embodiments, the status of the bay will appear in a dialog box and will update as the QC Cartridge passes through various stages: preflight, test started, in progress, result, etc.

If initialization (a.k.a. preflight) fails, the status dialog box displays “DNS” for “Does Not Start.” In some embodiments, when there is a preflight failure, the cartridge is ejected and the Days Until Due is not updated.

Processing a QC Cartridge can be further understood by the following numbered paragraphs:

Paragraph 1. A method for processing a QC Cartridge in a detection instrument, the method comprising:
receiving by the detection instrument scan data from a first QC Cartridge; receiving by the detection instrument the scanned first QC Cartridge into an available processing bay on the detection instrument; processing by the detection instrument the first QC Cartridge in the detection instrument.

Paragraph 2. The method for processing a QC Cartridge in a detection instrument of claim 1, further comprising: updating by the detection instrument the Days Until Due after the first QC Cartridge has been successfully processed.

Paragraph 3. The method for processing a QC Cartridge in a detection instrument of any preceding paragraph, wherein the Days Until Due for a second QC test are automatically updated after the first QC Cartridge is successfully processed.

Paragraph 4. The method for processing a QC Cartridge in a detection instrument of any preceding paragraph, wherein prior to processing the first QC Cartridge, the detection instrument correlates a cartridge Accession ID with a QC Rule.

Paragraph 5. The method for processing a QC Cartridge in a detection instrument of any preceding paragraph, wherein prior to processing the first QC Cartridge, the detection instrument correlates a cartridge Accession ID with an expected QC Rule.

Paragraph 6. The method for processing a QC Cartridge in a detection instrument of paragraph 5, wherein if the cartridge Accession ID does not match the expected QC Rule, the detection instrument processes the first QC Cartridge.

Paragraph 7. The method for processing a QC Cartridge in a detection instrument of paragraph 5, wherein if the cartridge Accession ID does not match the expected QC Rule, the detection instrument does not process the first QC Cartridge.

Paragraph 8. The method for processing a QC Cartridge in a detection instrument of any preceding paragraph, wherein if the first QC Cartridge is received into a reserved bay, supervisor approval is needed before the first QC Cartridge is processed in the reserved bay.

Paragraph 9. The method for processing a QC Cartridge in a detection instrument of any preceding paragraph, wherein if the first QC Cartridge is received into a reserved bay, the user must acknowledge the bay is reserved before the first QC Cartridge is processed in the reserved bay.

### Clinical Sample Runs

In some embodiments, when a regular clinical sample run is attempted, there is a warning if the corresponding external control is due and has not been successfully performed. In some embodiments, when a regular clinical sample run is attempted, the patient cartridge is not processed if the corresponding external control is due and has not been successfully performed.

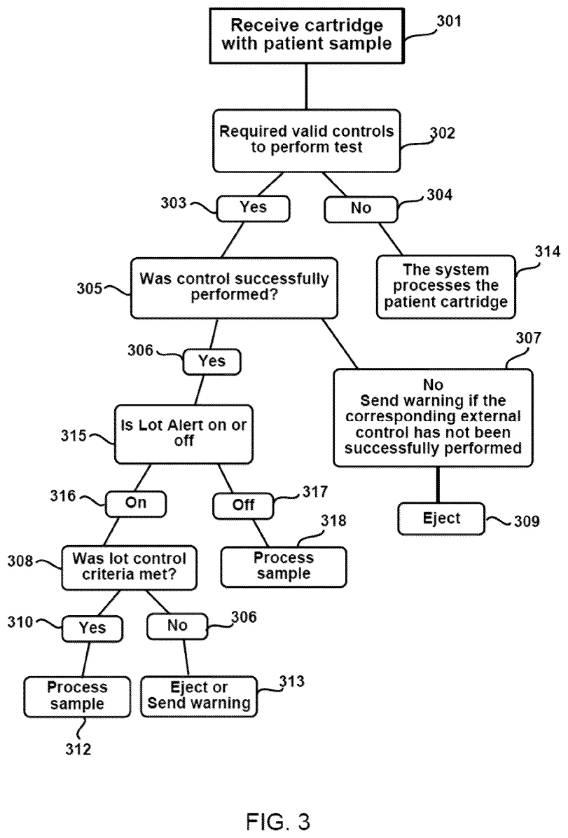

In some embodiments, when a Regular Run is attempted (i.e. a cartridge containing a clinical sample is loaded into the instrument, FIG. 3 at step 301), the system will check to see if the “required valid control(s) to perform tests” option is turned on. (FIG. 3 at steps 302, 303 and 304).

If the “required valid control(s) to perform tests” option is turned off (or is not on) (FIG. 3 at step 304), the processing instrument will process the clinical sample (FIG. 3 at step 314). If the “required valid control(s) to perform tests” option is turned off (or is not on) (FIG. 3 at step 304), the processing instrument will proceed to step 315 (lot change criteria on or off) (not shown in FIG. 3).

If the “required valid control(s) to perform tests” option is turned on (or is not turned off) (FIG. 3 at step 303) the processing instrument will determine if a control was successfully performed (FIG. 3 at step 305). A warning is issued if the corresponding external control has not been performed successfully within the number of days specified by the run frequency criterion (FIG. 3 at step 307). In some embodiments, the clinical sample will be ejected if the corresponding external control has not been performed successfully within the number of days specified by the run frequency criterion (FIG. 3 at step 309).

In some embodiments, when a Regular Run is attempted, it is prevented if the corresponding external control has not been performed successfully within the number of days specified by the Run Frequency Criterion and if the “Required valid control(s) to perform tests” option is turned on.

In some embodiments, when a Regular Run is attempted, if the corresponding external control has been performed successfully, the system checks if the Lot Alert is turned on (FIG. 3 at step 215). If the Lot Alert option is turned off (or is not on) (FIG. 3 at step 317), the processing instrument will process the clinical sample (FIG. 3 at step 318). If the Lot Alert option is turned on (or is not turned off) (FIG. 3 at step 316) the processing instrument will determine if the Lot Control Criteria was met (FIG. 3 at steps 308, 310 and 311). The Lot Control Criteria means the criteria to evaluate if the cartridge is from a lot that has had a valid QC test run, i.e., was the QC run performed successfully, within an acceptable timeframe, etc. For example, if the QC Cartridge for a first target was approved for a first lot but the Sample Cartridge is from a second different lot, the system may issue a Lot Alert. In some embodiments, the Lot Alert is activated when a user attempts to process a Sample Cartridge.

In some embodiments, if the QC Cartridge for a first target was approved for a first lot for a first lot approval time period and the Sample Cartridge is from the same first lot but is outside the lot approval time period, the system may issue a Lot Alert. In some embodiments, the Lot Alert is activated when a Sample Cartridge exceeds the lot approval time period.

If the corresponding QC test has not been performed successfully within the number of days specified by the Lot Control Criteria (FIG. 3 at step 311), a warning is issued or the cartridge is ejected (FIG. 3 at step 313). If the corresponding QC test has been performed successfully within the number of days specified by the Lot Control Criteria, the cartridge is processed (FIG. 3 at step 312).

In some embodiments, when a Regular Run is attempted, it is prevented if both the Lot Alert and the “Required valid control(s) to perform tests” option are turned on, and the criteria for each is not met.

In some embodiments, when a Regular Run is attempted, it is prevented if the Lot Alert or the “Required valid control(s) to perform tests” option are turned on, and the criteria for either is not met.

In some embodiments, when a patient sample is processed the system first evaluates if the Requires a Valid Control Option is turned on or off and then evaluates if the Lot Alert is on or off. In some embodiments, when a patient sample is processed the system first evaluates if the Lot Alert is on or off and then evaluates if the Requires a Valid Control Option is turned on or off.

In some embodiments, if the user selects a QC test to run, the Run View prompts the user to scan the cartridge that corresponds to the selected QC test.

Processing a Sample Cartridge can be further understood by the following numbered paragraphs:

Paragraph 1. A method for processing a Sample Cartridge in a detection instrument, the method comprising:
evaluating by the detection instrument whether a QC Control has been run for the same cartridge type as the Sample Cartridge; evaluating by the detection instrument whether the lot control criteria is met if the QC Control has been run for the Sample Cartridge type; processing the Sample Cartridge in the detection instrument if the lot control criteria was met.

Paragraph 2. A method for processing a Sample Cartridge in a detection instrument, the method comprising:
evaluating by the detection instrument whether a QC Control has been run for the same cartridge type as the Sample Cartridge; evaluating by the detection instrument whether the lot control criteria is met if the QC Control has been run for the Sample Cartridge type; ejecting the Sample Cartridge from the detection instrument if the lot control criteria was not met

Paragraph 3. A method for processing a Sample Cartridge in a detection instrument, the method comprising:
evaluating by the detection instrument whether a QC Control has been run for the same cartridge type as the Sample Cartridge; and ejecting by the detection instrument the Sample Cartridge from the detection instrument if the QC Control has not been run for the same cartridge type as the Sample Cartridge.

Paragraph 4. A method for processing a Sample Cartridge in a detection instrument, the method comprising:
receiving by the detection instrument a scanned Sample Cartridge; evaluating by the detection instrument whether the Sample Cartridge requires a QC test; evaluating by the detection instrument whether a QC test has been performed for the Sample Cartridge; evaluating by the detection instrument whether the lot control criteria is met for the Sample Cartridge; processing by the detection instrument the Sample Cartridge in the detection instrument if the lot control criteria was met.

Paragraph 5. The method for processing a Sample Cartridge in a detection instrument of paragraphs 1 and 4, wherein the Days Until Due for a first QC test are automatically updated by the detection instrument when the Sample Cartridge is processed.

### Reminders

Once QC Rules are scheduled, in one embodiment, the reminder module 509 automatically generates reminders for upcoming due dates. For example, if a QC run is approaching, then the reminder module 512 will notify the user a specified number of days/weeks ahead of time, so that the user has time to process the QC run. The specific timing of the reminder notifications may be specified by the end user and stored when creating or managing a QC Rule.

In some embodiments, the instrument automatically generates reminders for the end user, either in the form of an email which is sent to the user's email, text message, instant message, or to a pop-up window in the GUI. In reminders, upcoming due dates are listed in chronological order and, for each reminder, links are provided to generate a subsequent reminder (e.g., “remind me again in 3 days”). In addition, for each reminder, a “process QC Cartridge” link may be provided which, when selected, initiates the External Control Run described in FIG. 2. In addition, for each reminder, a “Disregard” link may be provided which, when selected, deletes the QC run from the schedule and updates the Days Until Due.

In one embodiment, the reminder list is ordered chronologically, with the nearest approaching due dates at the top of the list. Alternatively, in one embodiment, the reminder list may order a portion of the QC Rules chronologically but may also prioritize certain QC Rules and place them at the top of the list (notwithstanding the chronology). For example, in one embodiment, all respiratory QC Controls may be placed at the top of the reminder list when, for example, there is a respiratory outbreak in the hospital/region and the laboratory wants to ensure the instrument is not down for QC compliance so that it can process as many respiratory samples as possible. Similarly, the user may designate certain QC Rules as “high priority” (e.g., a QC ule which has a QC Cartridge that is about to expire, etc.). The reminder module 509 will then place any entries designated “high priority” at the top of the list (i.e., when the expiration date associated with the QC Cartridge is within a certain window of time). In one embodiment, any of the variables associated with the QC Cartridge or QC Rule may be used to prioritize the reminder list.

In one embodiment, only QC Rules due on the current day or future dates will be shown in the reminder list. Past due QC Rules are not displayed. In one embodiment, QC Rules due on the current day or future dates will be shown in the reminder list as well as past-due QC Rules.

An “add a QC Rule” button is also provided in the reminder to enable the user to add a new QC Rule to the reminder list.

Following step 102.5, in some embodiments, there is an additional stem for reminder scheduling, which allows a user to create a reminder schedule for the particular QC Rule. Following step 103.6, in some embodiments, there is an additional stem for reminder scheduling, which allows a user to set or to modify a reminder schedule for the particular QC Rule.

In some embodiments, the reminder stem includes a drop-down menu containing a set of predefined dates (1 week before due, 3 days before due, 1 day before due, etc.) or a date field that comprises a selectable calendar graphic for selecting a particular date.

In one embodiment, when a new QC Rule is created by a user, a reminder date it is automatically generated.

Processing a Sample Cartridge can be further understood by the following numbered paragraphs:

Paragraph 1. A method for managing quality control on a detection instrument, the method comprising, the method comprising:
displaying a reminder list on a GUI, wherein the reminder list comprises a plurality of reminder entries each identifying an upcoming event associated with one or more QC Rules.

Paragraph 2. A method for managing quality control on a detection instrument, the method comprising:
evaluating by the detection instrument whether a QC Control has been run;
decreasing the Days until Due by 1 for each QC Rule for which a QC Control has not been run; and
displaying a reminder list on a GUI for each QC Rule for which a QC Control has not been run.

### Days Until Due Update

There has been a successful External Control Run of the assay.

If the external control run is successfully completed, the Days Until Due is updated for that assay. (FIG. 2 at step 221). If the external control run is successfully completed, the Days Until Due are updated for other assays in the panel (not shown). The Days Until Due is only updated for a QC Rule if the external control run is successfully completed. The Days Until Due is not updated for a QC Rule if the external control run is not successfully completed.

If a control is scheduled to run daily for a first target, the Days Until Due for additional controls on the panel will be recalculated based on the date the most recent control was run.

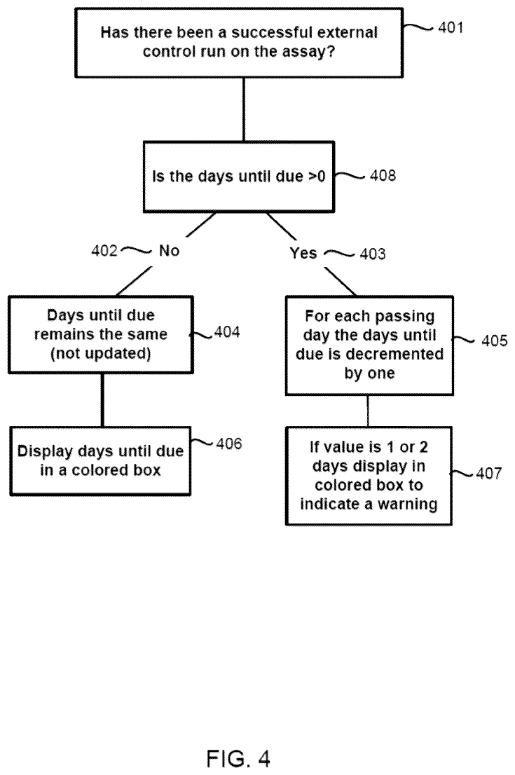

Each day, the processing instrument will assess whether there has been a successful External Control Run for each assay for which there is a QC Rule (FIG. 4 at step 401).

In some embodiments, the processing instrument will assess whether the Days Until Due is greater than zero (FIG. 4 at step 408).

In some embodiments, if the Days Until Due is greater than zero (FIG. 4 at step 403), for each passing day, the Days Until Due value is decremented by one (FIG. 4 at step 405). In some embodiments, if the Days Until Due value is 1 day or 2 days, it is displayed in a colored box, colored to indicate a warning to the user (FIG. 4 at step 407).

There has not been a successful External Control Run of the assay.

In some embodiments, if the Days Until Due is zero or less than zero (FIG. 4 at step 402), for each passing day, the Days Until Due remains the same (i.e., not updated) (FIG. 4 at step 404). In some embodiments, if the control is past due, it is displayed as overdue in a colored box to indicate it is past due (FIG. 4 at step 406).

In some embodiments, if the Days Until Due is zero or less than zero (FIG. 4 at step 402), for each passing day, the Days Until Due is increased by a negative number (not shown).

Rotation Order is ON and Frequency is ON and a successful control run has been completed

In some embodiments, the Days Until Due recalculates for all subsequent controls based on the actual run date of the completed control.

In some embodiments, if a control is run prior to its actual due date, the due dates of its successor will be moved up to reflect the new logical order of the controls within the existing sequence as well as for subsequent control sequences. For example, if the Days Until Due for a first target is 10 days and for a second target is 20 days and the first target QC run is run 3 days early, the Days Until Due for the second target is moved up by 3 days.

The initial intent of the customer based on the frequency input should not be broken, e.g. if a control is expected to run every month, then a month cannot be skipped simply because a control run was completed in sequence but too early. In some embodiments, if the Days Until Due for a first target is monthly and the first target QC run is run on the first day of a first month and the 25^{th} day of the first month, the Days Until Due will still reflect that the QC run must be run in the second month.

In some embodiments, if a control is run prior to its actual due date, the due dates of its successor will not be moved up. In some embodiments, if the Days Until Due for a first target is 10 days and for a second target is 20 days and the first target QC run is run 3 days early, the Days Until Due for the second target is not adjusted.

In some embodiments, the system suggests a rotation order but allow users to run controls out of order. In some embodiments, when a control is run out of order, the system now expects the next control in sequence based on the last successful control run. In some embodiments, when a control is run out of order, once a sequence is completed, the original order is resumed as expected.

### QC Control Alerts

The method further includes generating an alert in response to a QC Rule violation, i.e., when a QC Rule was due but was not run or was run but the actual result did not match the expected result. QC Control Alerts includes Expected Result Alerts and Past Due Alerts.

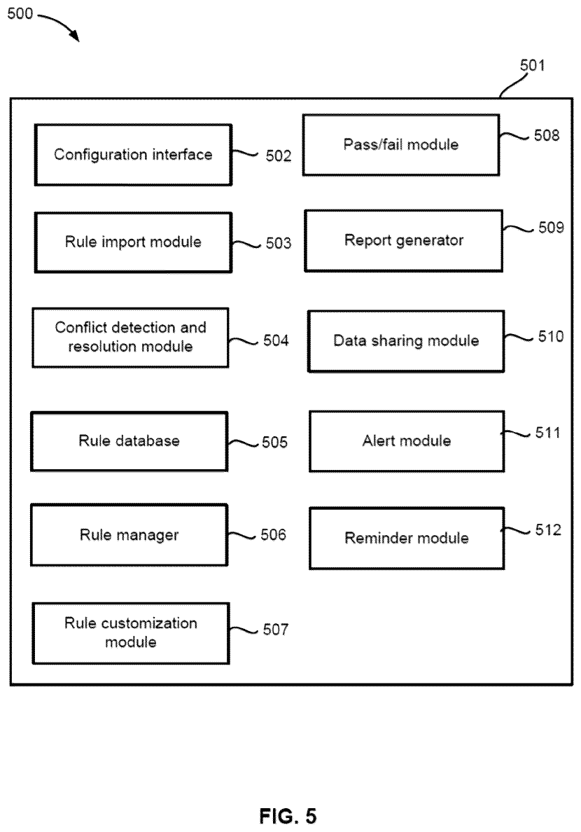

The QC Control Alert includes generating an alert and/or logging the violation in the alert module (FIG. 5 at 511). The alert can identify the rule broken, i.e., run frequency, run rotation, or Days Until Due violation. In some embodiments, the alert includes blocking the detection system from further processing any Sample Cartridges for an assay panel with a QC Control alert in place. In some embodiments, after a QC alert issues, the software saves the QC Control alert information such as username, date, timestamp, bay serial number, bay location, QC Rule violated, QC Cartridge serial number (if any), QC Cartridge Accession ID (if any).

QC Control Alerts may be applied generally in response to violated QC Rules as a whole or specifically for the violated rule such that different violated rules cause the instrument to perform different actions.

All alerts (QC Control alerts, QC result alerts, out-of-order run, Lot Alert, etc.) discussed herein can be presented to the user through a network accessible interface (i.e., GUI) or other forms of communication including email, text message, instant message, etc.

### Past Due Alert

The method further includes generating an alert in response to an alert being past due. For example, when a QC run is due on the first Monday of the Month and it is the first Tuesday of the Month the instrument will send a Past Due Alert to the user. In some embodiments, the Past Due alert is sent to a GUI. In some embodiments, the violation is logged in a QC alert module 511.

### Expected Result Alert

The method further includes generating an alert in response to comparing the predicted QC result with the actual QC result. For example, when the expected result is “Detected” but the actual result is “not Detected” the instrument will send a QC Result Alert to the user. In some embodiments, the QC result alert is sent to a GUI. In some embodiments, the violation is logged in a QC alert module 511.

### Summary Report

The method further includes generating a Summary Report of QC results. After the external control(s) for an assay panel are run, the report generator 509 generates a Summary Report. A Summary Report may be generated by a remote computing device or the processing instrument. The Summary Report identifies the QC tests run, the QC results for each target, any missing QC tests. The data sharing module 510 then shares the Summary Report by presenting it through a network accessible interface (i.e., GUI) or transmitting it through other forms of communication including email, text message, instant message, etc. The Summary Report may be a printout generated by a printing device (e.g., a printer) or a digital document that is viewed on a screen and/or securely transferred (e.g., via email, text message, instant message, etc.) to a healthcare professional, laboratory supervisor, FDA, etc.

### Quick View

In some embodiments, when a user selects a QC run from the QC Scheduler screen, they are presented with a “Quick View” option.

In some embodiments, the “Quick View” displays all relevant details of the control (run frequency, rotation order, Lot Alerts (on/off), Requires Valid Control (yes/no), Days Until Due, etc.) with added ability to export or print. In some embodiments, when export is selected, the Export File Explorer is presented to allow the user to select a destination. In some embodiments, when the Print icon is selected, a pull-up-list is presented to either Print Details or Print a summary of the QC rules.

### System

FIG. 5 is a schematic diagram of an example of a system 500 to aid laboratories manage QC requirements. The example of the system 500 shown in FIG. 5 includes an integrated diagnostic instrument 501, a rules configuration interface 502, QC Rule database 505 and a QC pass/fail module 508.

For example, a system implemented by a detection instrument allowing a user to customize QC requirements is described, the system comprising: a graphical user interface for customizing QC requirements comprising: a configuration interface 502. The configuration interface 502 may comprise a first GUI interface comprising the schedule of installed assay panels. The configuration interface 502 may comprise a second GUI interface comprising the schedule of targets on a panel. The configuration interface 502 may comprise a third GUI interface comprising an option to select a target run frequency. The configuration interface 502 may comprise a fourth GUI interface comprising an option to select a target rotation. The configuration interface 502 may comprise a fifth GUI interface displaying the Days Until Due.

The configuration interface 502 may comprise a second GUI interface comprising the schedule of targets on a panel and have sections for selecting a target run frequency, a target rotation, and displaying the Days Until Due.

In some embodiments, the GUI interface further comprises a list of specialized QC assays that are unique to the detection instrument, the laboratory, the hospital or geographic region of the laboratory/hospital.

In some embodiments, the GUI interface further comprises QC recommendations which are QC tests recommended for the detection instrument, the laboratory, the hospital or geographic region of the laboratory/hospital. The QC recommendations can be checked on or off from a drop-down menu.

In some embodiments, the instrument 501 comprises a QC Rule import module 503, which manages the importation of QC Rules from various local (laboratory) and/or remote (hospital) databases to the detection instrument. In the illustrated embodiment, the rules import module 503 comprises a conflict detection and resolution module 504.

The QC Rule import module imports QC Rules. The QC Rules may be imported from a local rule databases, remote database and/or existing rules already stored on the detection instrument 501. Once imported, the conflict detection and resolution module 504 compares the imported rule to existing rules already stored on the detection instrument 501 and resolves any conflicts. Stated another way, the conflict detection and resolution module 504 merges the new QC Rule with existing rules already stored on the detection instrument 501 and detects any conflicts which may result from the merge operation. A conflict may result if one or more QC Rule being imported are already stored within the existing QC database 505. In such a case, the conflict detection and resolution module 504 resolves the conflict using a set of conflict resolution rules (described below). Once all conflicts have been resolved, the data is persisted within the QC Rule database 505 and made accessible to end users via the QC Rule manager 506.

As mentioned above, a conflict detection and resolution module 504 identifies existing rules 505 which match rules being imported from the various sources and attempts to resolve any conflicts which may exist between the data. In one embodiment, each conflict is identified to the end user and the user is prompted to specify how to resolve the conflict. For example, the graphical user interface may show the user which rule data contained in the existing rules database 505 conflicts with rule data to be imported to allow the user to correct the conflict.

In one embodiment, the user is given the option to select the existing version 505 or the imported version.

In one embodiment, the various modules shown in FIG. 5 are not separate modules but, rather, separate data structures (e.g., tables) within a relational database.

In some embodiments, the user or the system identifies one or more “QC Classes” within the QC database 505. As described below, QC Classes are specialized groups of QC Rules which have similar application. The concept of a “QC Class” is a particularly useful abstraction for a detection instrument 501 which runs different types of assays such as Gram positive, Gram negative, fungal or respiratory assays. Some of these assays have unique QC Rules and some of these assays have the same QC Rules. For example, the gram-negative and gram-positive panels could have some targets which are the same. If a QC Class is generated, the target will be treated the same regardless of the assay panel it is on. Additionally, if a QC test is run for a target in a QC Class, the target can be shown to have undergone a QC test regardless of whether the target is on a Gram positive, Gram negative, fungal or respiratory assay. In one embodiment, a QC Class is formed when a QC Rules matches an existing rule and those rules have the same name AND one of the following: detect the same target or run the same control but are for different assay panels.

In some embodiments, to create a QC Class, the user selects and customizes a QC Rule. In one embodiment, this is accomplished with a QC Rule customization module 507. In one embodiment, the QC Rule customization module 507 automatically puts repetitive QC target rules for different assays into the same QC Class.

The system comprises a QC Rule customization module 507 for customizing QC test parameters. The customization module includes, for example, a drop down menu for selecting the assay panel, a target requiring QC on the panel, defining QC test run frequency, and defining QC test rotation. Alternatively, QC Rules may be imported from the QC Rule import module 503. Rules imported from the QC Rule import module 503 or defined in the QC Rule customization module 507 are processed by the conflict detection and resolution module 504.

QC Rules may be stored in a QC Rule database 505. After a QC test is processed, the QC pass/fail module 508 will assess and report if the QC test passed the QC requirements, e.g., detected the QC target when the expected result is detected and did not detect a QC target when the expected result is not detected. The reminder module 512 sends alerts that QC tests are upcoming.

Once a QC run is complete, the QC pass/fail module 508 determines if the detection instrument passed the QC Rule. If it passes, the pass/fail module 508 and/or alert module 511 notifies the user via a GUI pop-up or via email, text message, instant message, etc. If it fails, the pass/fail module 508 and/or alert module 511 sends a fail notification to the GUI or via email, text message, instant message, etc.

### Functional Specification

In some embodiments, the Run view (or Home screen) consists of the menu bar on the top, the body in the middle, and the info bar on the bottom. In some embodiments, the Menu bar allows the user to navigate to other areas of the application. In some embodiments, the system settings menu contains sub-menu items. In some embodiments, the info bar contains system information such as date and time, the logged in user and useful shortcuts such as Help, Screen Capture, etc. In some embodiments, the Body of the Home screen contains bay tiles at various states. In some embodiments, when the user closes a view from a different area (e.g., Test Results), they are brought back to the Home screen.

### Entering the Run View

In some embodiments, after the user logs in, a run view is displayed on the GUI.

In some embodiments, on the GUI, tiles which correspond to physical bays are displayed. In some embodiments, on the GUI, tiles which correspond to physical bays are displayed along with their status. Available bays are given a first color, bays processing a cartridge are given a second color, and disabled bays are given a third color, wherein the first, second, and third colors are different.

### Idle Bays

In some embodiments, when the user taps the Bay Settings icon, a Bay Settings dialog box appears which allows the user to turn on/off (enable/disable) that bay or to start a cleaning workflow.

In some embodiments, when the user changes the bay from on (enabled) to off (disabled), the reason text box appears, and a reason can be provided.

In some embodiments, when the user changes the bay from on (enabled) to off (disabled), a report can automatically be sent to Technical Support.

In some embodiments, the ISW pings a bay every so often. In some embodiments, the ISW pings a bay every day, every 5 hours, hour, half hour, 10 minutes, 5 minutes, 2 minutes, minute, 30 seconds, or 10 seconds, even when the bay is idle, to make sure the bay is alive.

In some embodiments, if a bay fails to respond to a ISW ping, the bay is marked as having a Bay Communication Failure. In some embodiments, if a bay has a Bay Communication Failure, the bay light is changed to a third color, indicating the bay is not available to use.

### Starting a Sample Run

In some embodiments, to start a run, the user selects an available bay icon and taps it. In some embodiments, when a user selects an available bay the GUI displays a dialog prompting the user to scan a patient cartridge.

In some embodiments, after the user scans a cartridge, if the cartridge Accession ID barcode is not present on the cartridge, the GUI displays a dialog allowing the user to manually enter the Accession ID. Stated another way GUI displays a dialog to receive user input of a manually entered Accession ID.

In some embodiments, if the Accession ID matches an external control ID, the system marks the run as an External Control Run.

In some embodiments, if both the Accession ID and the assay type match a PTO, the PTO entry in the database is deleted once the run completes successfully.

In some embodiments, after the cartridge is scanned, a dialog box appears in the GUI asking the user to select a dynamic panel to run.

In some embodiments, after the user scans a cartridge, if the cartridge information barcode is not present on the cartridge, the GUI displays a dialog allowing the user to manually enter cartridge information.

In some embodiments, if the “Require Valid Control to Perform Tests” option is on and there are no valid control runs for the assay the system prevents the user from processing the patient sample.

In some embodiments, if the “Require Valid Control to Perform Tests” option is off, the system auto-generates a comment if a sample run was performed and the external control was due but not required.

In some embodiments, after the cartridge is scanned, the available bay lights indicate to the user that they are ready to accept the scanned cartridge.

In some embodiments, if the user selects a bay from the GUI, the selected bay's light is changed to a first color (e.g. blinking white) to indicate that the bay is ready to accept the cartridge. In some embodiments, if the user selects a bay from the GUI, the selected bay's light is changed to a first color (e.g. blinking white) to indicate that the bay is ready to accept the cartridge, and the GUI displays a dialog prompting the user to insert the scanned cartridge into the selected ready bay. In some embodiments, if the user selects a bay from the GUI, the system notifies the selected bay to get into the ready-to-accept-cartridge state. In some embodiments, if the user selects a bay from the GUI, the system nevertheless tells all the available bays to get into the ready-to-accept-cartridge state, not just the selected bay.

In some embodiments, if the user does not select a bay from the GUI, the LED light of all available bays is changed (e.g., blinking white) to indicate that they are ready to accept a cartridge. In some embodiments, if the user does not select a bay from the GUI, the LED light of all available bays is changed (e.g., blinking white) to indicate that they are ready to accept a cartridge, and the GUI displays a dialog prompting the user to insert the scanned cartridge into any ready bay. In some embodiments, if the user does not select a bay from the GUI, the system tells all the available bays to get into the ready-to-accept-cartridge state.

In some embodiments, if the user selects a reserved bay and Supervisor approval is required, the GUI displays the sign-off dialog box for a Supervisor user to sign.

In some embodiments, if the user inserts the cartridge into a reserved bay and Supervisor approval is not required, the GUI displays an acknowledgement dialog box. The operator must acknowledge they are using a reserved bay by hitting an acknowledgement button or swiping a badge. If the operator does not acknowledge they are using a reserved bay by hitting an acknowledgement button or swiping a badge, the cartridge cannot be processed.

In some embodiments, after the user inserts a cartridge into a bay, the system displays a dialog showing “Initializing . . . ” (a.k.a. preflight). The system initializes the run.

The header reads “Cartridge Inserted”, the body reads Initializing, and the bay tile is colored to show “Initializing . . . ” and a progress bar in gray with a 2-minute duration (preflight time).

In some embodiments, after the user inserts a cartridge into a bay, the system displays a dialog box reading “Ready for next cartridge” to prompt the user to scan the next cartridge.

In some embodiments, if initialization (a.k.a. preflight) fails, the GUI displays DNS on the bay tile with the Reset Bay button and changes the LED light of that bay to a color indicating a preflight failure.

In some embodiments, if initialization succeeds, the LED light of that bay changes to a different color to indicate the cartridge is being processed.

### During a Run

In some embodiments, the assay tag is displayed on the bay tile. In some embodiments, the Accession ID is displayed on the bay tile. In some embodiments, the assay tag and the Accession ID are displayed on the bay tile.

In some embodiments, the QC tag is displayed on the bay tile. In some embodiments, the Lot ID is displayed on the bay tile. In some embodiments, the QC tag and Lot ID are displayed on the bay tile.

In some embodiments, the estimated run time is displayed on the bay tile. In some embodiments, the estimated completion time is displayed on the bay tile. In some embodiments, a progress bar is displayed on the bay tile. In some embodiments, estimated run time, estimated completion time, a progress bar, or combinations thereof, are displayed on the bay tile.

In some embodiments, if during a run, the user taps the bay tile, the GUI displays the Test Details showing test information and the Bay Details showing bay information.

### Ending a Run

In some embodiments, if a run ends before the estimated completion time due to a bay error, the GUI displays a DNF error in the bay tile. In some embodiments, if a run ends before the estimated completion time due to a bay error, the GUI displays a DNF error in the bay tile and the Bay's LED light is changed to a color to indicate the DNF.

In some embodiments, if a run ends approximately at the estimated completion time but the test result is invalid due to assay logic (not bay error), the GUI displays Invalid Test in the bay tile. In some embodiments, if a run ends approximately at the estimated completion time, but the test result is invalid due to assay logic (not bay error), the GUI displays Invalid Test in the bay tile and changes the bay's LED light to a color indicating the Invalid Test.

In some embodiments, if the user hits the Abort button in the Test Details view, the system will end the run as a DNF with a message “User aborted the test” in the Detection Report.

In some embodiments, if a run ends approximately at the estimated completion time without any errors, the GUI displays the number of detected targets (zero or more). In some embodiments, if a run ends approximately at the estimated completion time without any errors, the GUI displays the number of detected targets (zero or more) and the bay's LED light is changed to a color indicating the run is complete.

In some embodiments, if the run was a PTO run, the system marks that PTO run in the database as “complete.” In some embodiments, if the run was a PTO run, the system reports the result.

In some embodiments, if the run was a QC (external control) run, the system updates the Days Until Due of the corresponding external controls of that assay.

In some embodiments, if a run ends approximately at the estimated completion time without any errors, but there's an LIS hold (due to LIS advanced rules), an alert is sent to the user but the result is not automatically sent to LIS. The alert may be a blinking tile on the screen and/or LED light on the bay. In some embodiments, when a result is subject to an LIS hold, the user can tap the bay icon to view the details of the result, hold, detection report, and/or combinations thereof. In some embodiments, when a result is subject to an LIS hold, the user can manually send the result to the hospital's' LIS.

ISW will follow the following workflow to address the DNF's:
1. Cartridge started
2. bay failure occurs
3. Cartridge ejects
4. Bay enters error state
5. Cartridge is removed
6. Bay auto-POSTS
7. Bay enters ready state

ISW will attempt auto-POST when Bay encounters error. Bay enters to ready state if error is resolved by Auto-POSTs, otherwise Bay shows failure state. ISW will record failures in audit log/system event.

### Removing a Cartridge

In some embodiments, after the user removes a cartridge from a bay and, if there were no bay errors (e.g., user aborted run, invalid test) on that bay, the bay's LED color is changed to indicate that it can accept a new cartridge.

In some embodiments, after the user removes a cartridge from a bay, if there was a DNF, the bay is disabled automatically. The bay's LED light is changed to a color indicating that the bay is disabled due to the run history on that bay.

### System Restart Before Cartridge Removal

Sometimes the system restarts (turned off, then on), before the cartridge is removed. The software needs to restore the bay state to before the system was off, when the system comes back on.

In some embodiments, if a bay without a cartridge was in the ready state when the system was turned off, the system will put the bay in the ready state upon restart.

In some embodiments, if a bay with a cartridge was in the successful state (green) when the system was turned off, the system shall put the bay in the successful state (green) upon restart.

In some embodiments, if a bay with a cartridge was in the error state (red) when the system was turned off, the system shall put the bay in the error state (red) upon restart.

### Bay OQ Workflow

Operational Qualification is performed on bays as part of installation and/or troubleshooting. In such situations, the entire instrument must be idle—no runs processed.

The analysis devices is configured such that Patient samples can be in progress and/or started while Operational Qualification is running. Operational Qualification failed tests will be displayed inside ISW—no need to toggle (interrupt patient runs) between desktop and ISW.

Operational Qualification (“OQ”) includes the tests to be run in order to verify that the software is operationally fit to be deployed to consumers. Ideally, the key functionalities of the software are verified as part of this validation process. OQ follows a comprehensive, well-defined protocol aimed at ensuring the system is functioning according to pre-set and validated operational specifications. The OQ protocol was developed and validated internally and is updated following each instrument hardware and software release, ensuring the laboratory is receiving the most up-to-date service. Critical aspects of the OQ include but are not limited to: motion qualification, optics qualification, fluidics qualification, and thermal qualification. OQ is typically performed after a reactive service, software upgrade, or preventive maintenance, periodically, according to lab standard operating procedure.

In some embodiments, a bay is marked in the GUI or via its LED light as requiring OQ. This means that the bay itself needs to undergo operational qualification.

In some embodiments, a bay marked for OQ can only run OQ operations, not any other kind of run (i.e., sample, QC external control, Cleaning Card, etc.). In some embodiments, a bay marked for OQ can run other operations.

In some embodiments, if a bay is marked for OQ and a user scans a non-OQ card, the GUI displays an error and the system stops the run.

In some embodiments, if a bay is marked for OQ, the user scans a Bay OQ Card and loads it into the bay. The system will process the Bay OQ Card.

In some embodiments, a bay must undergo operational qualification according to a particular frequency (Weekly, Monthly, or Custom). In some embodiments, the bay operational qualification frequency is defined by the laboratory, and in some embodiments, the bay operational qualification frequency is defined by the diagnostic instrument provider or both.

In some embodiments, if a bay OQ is due, the bay is disabled until it has successfully processed a Bay OQ Card. In some embodiments, after bay operational qualification, the bay is reenabled.

In some embodiments, the GUI has a Bay OQ Button and the user taps the Bay OQ Button and then selects the bay to undergo operational qualification. In some embodiments, the user selects the bay icon on the GUI and the GUI then displays various details about the bay, such as the last run time, whether the bay is reserved, the last time the bay was cleaned, the last time the bay underwent operational qualification. The bay details will also have a Bay OQ Button the user can tap. In some embodiments, any bay can undergo operational qualification at any time. In some embodiments, only bays that have been automatically disabled can undergo operational qualification. In some embodiments, only bays scheduled for operational qualification can accept a Bay OQ Card. The Bay OQ details can be exported as a report.

In some embodiments, the GUI has a Bay OQ Button and the user taps the Bay OQ Button and then selects the bay to undergo operational qualification. Before operational qualification begins, the user must select a reason for OC. The user can select a reason from a dropdown or enter a reason in free text. Dropdown reasons include IQCP, Maintenance, Verification, Other. If the user selects Other, they may input a free text reason for QC.

Once a user taps the Bay OQ Button, the bay's LED light changes to indicate that the bay can accept the Bay OQ Card. In some embodiments, only bays that have had their Bay OQ Button pressed can accept a Bay OQ Card. In some embodiments, any bay can accept a Bay OQ Card at any time.

In some embodiments, to initiate bay operational qualification, the user scans a bar code on the Bay OQ Card. In some embodiments, to initiate operational qualification, the user scans a bar code on the Bay OQ Card, and the user can insert the Bay OQ Card into any available bay. In some embodiments, to initiate operational qualification, the user scans a bar code on the Bay OQ Card, and the user can insert the Bay OQ Card into any bay whose light has been changed to indicate it can accept a Bay OQ Card.

In some embodiments, the Bay OQ Card has a single bar code wherein the single bar code identifies the Bay OQ Card as an operational qualification card. In some embodiments, the Bay QC Card has two bar codes wherein both bar codes identify the Bay OQ Card as a Bay OQ Card.

In some embodiments, if the user taps the Bay OQ Button, but then scans a cartridge which is not a Bay OQ Card, the system stops the Bay OQ Run. In some embodiments, if the user taps the Bay OQ Button, but then scans a cartridge which is not a Bay OQ Card, the system stops the run and then puts the bay into a ready-to-accept-cartridge state. The ready-to-accept-cartridge state means that the bay is able to accept any cartridge: Sample Cartridge, QC cartridge, Bay OQ Card, or Cleaning Card.

In some embodiments, the Bay OQ Card has a single bar code, wherein the single bar code identifies the Bay OQ Card as an operational qualification card. In some embodiments, the Bay OQ Card has two bar codes, wherein both bar codes identify the Bay OQ Card as a Bay OQ Card.

In some embodiments, when the Bay OQ Card is in process, the GUI (bay tile) indicates that the bay is processing an OQ Card. In some embodiments, when the Bay OQ Card has been successfully performed, the GUI (bay tile) indicates that the bay has passed OQ testing. In some embodiments, when the Bay OQ Card has not been successfully performed, the GUI (bay tile) indicates that the bay has failed OQ testing.

### Reserve Bay for Repair

In some embodiments, bays can be reserved for repair while the system is in use. When a bay is reserved and a user scans a cartridge, the reserved bay will not be identified (bay tile will not light up, bay door will not light up) as available and thus will not accept a cartridge, even while the rest of the system will.

Just as with reserving bays for “STAT” test, bays can be reserved for repair. The analysis device can allow customers the ability to reserve a single bay or a desired number of bays. In some embodiments, the analysis device requires a password to be entered prior to using a bay reserved for OQ. The reserved OQ bay(s) are reserved based on location, not availability. Thus, a particular bay is reserved not just any bay on the system. Thus, is bay 1 and bay 2 are reserved in a 6 bay system, bays 1 and 2 cannot process a sample cartridge or QC cartridge. If two bays are reserved for “STAT” testing any of the remaining 4 available bays can be used to process a clinical sample. But, if a user tries to enter a 3^{rd} sample cartridge or QC cartridge, the remaining 2 bays will enter a reserved state, i.e., a sample or QC cartridge will not be processed unless the reservation is bypassed.

In some embodiments, if the user inserts the cartridge into a OQ reserved bay, the cartridge will be ejected. In some embodiments, if the user inserts the cartridge into a OQ reserved bay, the system will allow the bay to process the cartridge if super visor approval is provided. A Supervisor will need to enter a unique code to process the cartridge in a reserved bay or swipe a badge to give authorization.

Reserving a bay can be further understood by the following numbered paragraphs:

Paragraph 1. A method managing a detection instrument comprising bays, the method comprising: determining, by the detection device that a first bay should be reserved; reserving the first bay so that it will not process any cartridges; processing a first patient sample in a second bay.

Paragraph 2. The method of Paragraph 1, wherein determining, by the detection device that a first bay should be reserved comprises applying a set of configurable rules to the first bay that define if the first bay requires OQ or if the first bay is reserved for STAT orders.

Paragraph 3. A pathogen detection device comprising: a reserve bay decision circuit configured to determine if a first bay should be reserved; a reserve bay implementation circuit configured to reserve the first bay wherein reserving the first bay comprises not displaying the first bay in a GUI, not illuminating a first bay door light or locking the first bay door.

Paragraph 4. The pathogen detection device of paragraph 3, wherein the reserve bay decision circuit is configured to allow a user to modify any reserve bay configurable rules via a graphical user interface.

Paragraph 5. The pathogen detection device of Paragraph 3, further comprising a reserve bay rule reconciler that is configured to automatically compare a new reserve bay rule to an existing reserve bay rule in reserve bay rule storage.

Paragraph 6. The pathogen detection device of Paragraph 5, wherein if the reserve bay rule reconciler identifies a conflict between a new reserve bay rule to an existing reserve bay rule in rule storage, the new reserve bay rule is sent to a rule organizer management module.

Paragraph 7. The pathogen detection device of Paragraph 3, wherein the reserve bay configurable rules are established by a user selecting from a series of selectable data elements to define if a bay should be reserved.

Paragraph 8. The pathogen detection device of Paragraph 3, wherein when a reserve bay rule is evaluated a first reserve bay rule is considered first and then any rule children associated with the first reserve bay rule; a second reserve bay rule is considered second and then any rule children associated with the reserve bay rule.

Paragraph 9. A non-transitory computer readable storage medium comprising computer executable instructions, the computer executable instructions executable by a processor, the computer executable instructions comprising: instructions executable to reserve a first bay; and instructions executable to enable a second bay when the first bay is reserved.

Paragraph 10. A pathogen detection device comprising: a circuit configured to display a graphical user interface to accept user input and change a first bay from enabled to reserved in response to the user input.

Paragraph 11. A pathogen detection device comprising: a display device; and a processor configured to:

display, on the display device, a graphical user interface that provides a field for user input; disabling a first bay based on the user input; processing a cartridge in a second bay.

Paragraph 12. The pathogen detection device of Paragraph 11, wherein the graphical user interface provides the field using a drop-down menu to receive user input.

Paragraph 13. A pathogen detection device comprising: at least one bay; a display device; and a processor configured to: display, on the display device, a graphical user interface that provides a drop-down menu of a plurality of pre-populated choices for user selection; receive a user selection of a pre-populated choice;

create a rule, wherein the created rule defines the scope of a reserve bay rule; apply the reserve bay rule to the at least one bay; and in response to the rule being satisfied, reserves the bay.

Paragraph 14. The pathogen detection device of Paragraph 13, wherein the reserve bay rule defines if the bay is enabled or disabled.

Paragraph 15. The pathogen detection device of Paragraph 13, wherein the reserved bay rule comprises a condition for applying the rule, and wherein the processor is further configured to define when a condition is met.

Paragraph 16. The pathogen detection device of Paragraph 15, wherein the conditions comprise one or more of the following: a particular time period, a particular account, a particular bay, a particular reserved bay requirement, or a particular QC requirement.

### Disable Bay

In some embodiments, when a run fails on a particular bay, the user can disable the bay. In some embodiments, when a run fails on a particular bay, the user can resent the bay. In some embodiments, when a run fails on a particular bay, the user can now disable or reset that bay directly in the Run View (or home screen) screen. In some embodiments, the GUI (bay tile) has a disable bay button to disable the bay. In some embodiments, the disabled bay button can only be selected if the bay processed a cartridge that had a DNF or DNS error. In some embodiments, the disabled bay button can be selected at any time. In some embodiments, when the user taps the Bay Settings icon, a Bay Settings dialog box appears which allows the user to turn on/off (enable/disable) that bay.

In some embodiments, when a bay is disabled, it cannot accept any cartridges (sample cartridges, QC cartridges, cleaning cartridges, etc.)

In some embodiments, the system 500 shown in FIG. 5 includes an integrated diagnostic instrument 501, a rules configuration interface 502, a disable bay Rule database (part of 505) and a reserve bay Rule database (part of 505).

For example, a system implemented by a detection instrument allowing a user to customize bay availability is described, the system comprising: a graphical user interface for customizing disable bay requirements and reserve bay requirements comprising: a bay configuration interface. The bay configuration interface may comprise a first GUI interface comprising the schedule of available bays. The bay configuration interface may comprise a second GUI interface comprising an option to reserve or disable a bay based on a plurality of selectable parameters. The bay configuration interface may comprise a third GUI interface comprising an option to select a bay to be reserved or disabled based on a defined rotation. The bay configuration interface may comprise a fourth GUI interface displaying the Days Until Enabled which defines when the bay will be re-enabled based on the defined rotation.

The bay configuration interface may comprise a second GUI interface comprising the schedule of OC Rule testing and have sections for selecting a bay run frequency, a bay rotation, and displaying the Days Until Enabled.

In some embodiments, the GUI interface further comprises a list of specialized OQ rules that are unique to the detection instrument, the laboratory, the hospital or geographic region of the laboratory/hospital.

In some embodiments, the GUI interface further comprises OQ testing recommendations which are OQ tests recommended for the detection instrument, the laboratory, the hospital or geographic region of the laboratory/hospital. The OQ testing recommendations can be checked on or off from a drop-down menu.

In some embodiments, the instrument 501 comprises a OQ Rule import module (503) which manages the importation of OQ Rules from various local (laboratory) and/or remote (hospital) databases to the detection instrument. In the illustrated embodiment, the rules import module (503) comprises a conflict detection and resolution module (504).

The OQ Rule import module 503 imports OQ Rules. The OQ Rules may be imported from a local rule databases, remote database and/or existing rules already stored on the detection instrument 501. Once imported, the conflict detection and resolution module (504) compares the imported rule to existing rules already stored on the detection instrument 501 and resolves any conflicts. Stated another way, the conflict detection and resolution module (504) merges the new OQ Rule with existing rules already stored on the detection instrument 501 and detects any conflicts which may result from the merge operation. A conflict may result if one or more OQ Rule being imported are already stored within the existing OQ database (505). In such a case, the conflict detection and resolution module (504) resolves the conflict using a set of conflict resolution rules (described below). Once all conflicts have been resolved, the data is persisted within the OQ Rule database 505 and made accessible to end users via the OQ Rule manager (506).

As mentioned above, a conflict detection and resolution module 504 identifies existing OQ rules which match rules being imported from the various sources and attempts to resolve any conflicts which may exist between the data. In one embodiment, each conflict is identified to the end user and the user is prompted to specify how to resolve the conflict. For example, the graphical user interface may show the user which OQ rule data contained in the existing rules database conflicts with OQ rule data to be imported to allow the user to correct the conflict. In one embodiment, the user is given the option to select the existing version or the imported version.

The system comprises a OQ Rule customization module (507) for customizing OQ test parameters. The customization module includes, for example, a drop down menu for selecting the bay requiring OQ, defining OQ test run frequency, and defining bay rotation. Alternatively, OQ Rules may be imported from the OQ Rule import module 503. Rules imported from the OQ Rule import module 503 or defined in the OQ Rule customization module (507) are processed by the conflict detection and resolution module 504.

OQ Rules may be stored in a OQ Rule database 505. After a OQ test is processed, the OQ Pass/fail module (508) will assess and report if the OQ test/bay passed the OQ requirements. The reminder module 512 sends alerts that OQ tests are upcoming.

Once a OQ run is complete, the OQ Pass/fail module (508) determines if the bay passed the OQ Rule. If it passes, the pass/fail module and/or alert module notifies the user via a GUI pop-up or via email, text message, instant message, etc. If it fails, the pass/fail module and/or alert module sends a fail notification to the GUI or via email, text message, instant message, etc.

Disabling a bay can be further understood by the following numbered paragraphs:

Paragraph 1. A method managing a detection instrument comprising bays, the method comprising: determining, by the detection device that a first bay should be disabled; disabling the first bay so that it will not process any patient samples; processing a first patient sample in a second bay.

Paragraph 2. A method managing a detection instrument comprising bays, the method comprising: processing a first cartridge in a first bay wherein the first cartridge does not run to completion; disabling the first bay so that it will not process any patient samples; processing a second cartridge in a second bay.

Paragraph 3. A pathogen detection device comprising: a disable bay decision circuit configured to determine if a first bay should be disabled; a disable bay implementation circuit configured to disable the first bay wherein disable the first bay comprises not displaying the first bay in a GUI, not illuminating a first bay door light or locking the first bay door.

Paragraph 4. The pathogen detection device of paragraph 3, wherein the disable bay decision circuit is configured to allow a user to modify any reserve bay configurable rules via a graphical user interface.

Paragraph 5. The pathogen detection device of Paragraph 3, further comprising a disable bay rule reconciler that is configured to automatically compare a new disable bay rule to an existing disable bay rule in disable bay rule storage.

Paragraph 6. The pathogen detection device of Paragraph 5, wherein if the disable bay rule reconciler identifies a conflict between a new disable bay rule to an existing disable bay rule in disable rule storage, the new disable bay rule is sent to a rule organizer management module.

Paragraph 7. The pathogen detection device of Paragraph 3, wherein the disable bay configurable rules are established by a user selecting from a series of selectable data elements to define if a bay should be disable.

Paragraph 8. The pathogen detection device of Paragraph 3, wherein when a disable bay rule is evaluated a first disable bay rule is considered first and then any rule children associated with the first disable bay rule; a second disable bay rule is considered second and then any rule children associated with the disable bay rule.

Paragraph 9. A non-transitory computer readable storage medium comprising computer executable instructions, the computer executable instructions executable by a processor, the computer executable instructions comprising: instructions executable to disable a first bay; and instructions executable to enable a second bay when the first bay is disabled.

Paragraph 10. A pathogen detection device comprising: a circuit configured to display a graphical user interface to accept user input and change a first bay from enabled to disabled in response to the user input.

Paragraph 11. A pathogen detection device comprising: a display device; and a processor configured to:

display, on the display device, a graphical user interface that provides a field for user input; disable a first bay based on the user input; process a cartridge in a second bay.

Paragraph 12. The pathogen detection device of Paragraph 11, wherein the graphical user interface provides the field using a drop-down menu to receive user input.

Paragraph 13. A pathogen detection device comprising: at least one bay; a display device; and a processor configured to: display, on the display device, a graphical user interface that provides a drop-down menu of a plurality of pre-populated choices for user selection; receive a user selection of a pre-populated choice; create a rule, wherein the created rule defines the scope of a disabled bay rule; apply the disabled bay rule to the at least one bay; and in response to the rule being satisfied, disable the bay.

Paragraph 14. The pathogen detection device of Paragraph 13, wherein the disable bay rule defines if the bay is enabled or disabled.

Paragraph 15. The pathogen detection device of Paragraph 13, wherein the disable bay rule comprises a condition for applying the rule, and wherein the processor is further configured to define when a condition is met.

Paragraph 16. The pathogen detection device of Paragraph 15, wherein the conditions comprise one or more of the following: a particular time period, a particular account, a particular bay, a particular disable bay requirement, a particular OC requirement, a prior failed run, or a particular QC requirement.

### Que Bay for OQ

In some embodiments, a user can queue an individual bay for running an OQ test (instead of turning off the entire system).

In some embodiments, the user can select a Bay OQ Test Frequency.

In the bay tile, the user selects a Bay OQ Test Frequency for the selected bay (Weekly, Monthly, or Custom). In some embodiments, the system automatically assigns a Bay OQ Test Frequency to each bay. In some embodiments, to establish a custom Bay OQ Test Frequency, the user selects a frequency which may be every X number of days, where X=1 to 365 days. In some embodiments, to establish a custom Bay OQ Test Frequency, the user selects a frequency which may be every X number of days, where X=1 to 100 days. In some embodiments, to establish a custom Bay OQ Test Frequency, the user selects a frequency, which may be monthly. In some embodiments, to establish a custom Bay OQ Test Frequency, the user selects a frequency, which may be weekly. In some embodiments, to establish a custom Bay OQ Test Frequency the user selects a frequency which may be every the first, second, third, fourth or fifth Monday, Tuesday, Wednesday, Thursday, Friday, Saturday or Sunday of the selected month(s).

In some embodiments, all of the bays on the analysis device have the same Bay OQ Test Frequency. In some embodiments, all of the bays on the analysis device have a different Bay OQ Test Frequency. In some embodiments, a first plurality of bays have a first Bay OQ Test Frequency and a second different plurality of bays have a second Bay OQ Test Frequency. In some embodiments, the first run frequency and second run frequency are different.

In some embodiments, each bay on the analysis device has a Bay OQ Test Frequency rotation.

In some embodiments, the system automatically assigns a rotation order to the bays on the analysis device. In some embodiments, in the user selects a rotation for each bay. To assign the bay a rotation order the user inserts a rotation order into the rotation order selection in the GUI interface.

In some embodiments, to assign the bay a Bay OQ Test Frequency rotation order, the user manually enters a rotation order into the rotation order selection box, i.e., first, second, third, etc. In some embodiments, by default, the rotation order selection boxes are empty, and the user fills them in. In some embodiments, by default, the rotation order selection boxes are filled in, and the user only changes the rotation order if desired. In some embodiments, to assign the bay a Bay OQ Test Frequency rotation order, the user selects from the available rotation orders in a rotation order dropdown wherein the number of available rotation orders matches the number of bays on the analysis system.

Once Bay OQ Test Frequency are scheduled, in one embodiment, the Bay OQ Test Frequency reminder module automatically generates reminders for upcoming due dates. For example, if a Bay OQ Test is approaching, then the Bay OQ Test Frequency reminder module will notify the user a specified number of days/weeks ahead of time, so that the user has time to process the Bay OQ Test. The specific timing of the reminder notifications may be specified by the end user and stored when creating or managing a Bay OQ Test Frequency.

In some embodiments, the instrument automatically generates Bay OQ Test Frequency reminders for the end user, either in the form of an email which is sent to the user's email, text message, instant message, or to a pop-up window in the GUI. In reminders, upcoming due dates are listed in chronological order and, for each reminder, links are provided to generate a subsequent reminder (e.g., “remind me again in 3 days”). In addition, for each reminder, a “process Bay OQ Card” link may be provided which, when selected, initiates the Bay OQ Test. In addition, for each reminder, a “Disregard” link may be provided which, when selected, deletes the Bay OQ Test from the schedule.

In one embodiment, the reminder list is ordered chronologically, with the nearest approaching due dates at the top of the list. Alternatively, in one embodiment, the reminder list may order a portion of the Bay OQ Test chronologically but may also prioritize certain bays and place them at the top of the list (notwithstanding the chronology). For example, users tend to use the bay in the top left hand corner. The system can prioritize OQ testing for that bay to ensure it is always available for use and is not down for OQ testing compliance so that it can process as many cartridges as possible pursuant to user preference.

In one embodiment, only Bay OQ Test due on the current day or future dates will be shown in the reminder list. Past due Bay OQ Test are not displayed. In one embodiment, Bay OQ Tests due on the current day or future dates will be shown in the reminder list as well as past-due Bay OQ Tests.

In one embodiment, when a new Bay OQ Test is created by a user, a reminder date it is automatically generated.

Paragraph 1. A method for managing a detection instrument, the method comprising: displaying a reminder list on a detection instrument GUI, wherein the reminder list comprises a plurality of reminder entries each identifying an upcoming event associated with one or more OQ tests.

Paragraph 2. A method for managing a detection instrument, the method comprising, the method comprising:
evaluating by the detection instrument whether a OQ Rule has been satisfied; decreasing the Days until Due by 1 for each OQ Rule for which a OQ test has not been run; and displaying a reminder list on a GUI for each OQ Rule for which a OQ test has not been run.

### Scan Protocol

In some embodiments, the system has a modified scan protocol. If the user scans the bar code and a bar code for the cartridge ID is present but a bar code for the accession ID is not present, the system will prompt the user to manually add an accession ID. Generally, the system expects to see two tags (accession ID and assay identification) each time it scans a sample cartridge. If, when a user scans the sample cartridge, only one tag is detected, the user must manually move to a separate manual mode to enter information. Now, if only one tag is detected, the system now automatically moves the user to a manual mode so that the user can enter information such as cartridge ID and/or accession ID information.

Scan protocol can be further understood by the following numbered paragraphs:

Paragraph 1. A method for receiving a cartridge by a diagnostic system, the method comprising: receiving a first scan bar code data; comparing the first scan bar code data to the expected scan bar code data; determining that the first scan bar code data does not match the expected scan bar code data; displaying on a display device, a graphical user interface that provides a field for user input; receiving user input which reconciles the first scan bar code data with the expected scan bar code data thereby receiving a cartridge.

Paragraph 2. The method for receiving a cartridge by a diagnostic system of paragraph 1, wherein the user input is second scan bar code data.

Paragraph 2.1. The method for receiving a cartridge by a diagnostic system of paragraph 1, wherein the first scan bar code data is cartridge data and the second scan bar code data is Accession ID data.

Paragraph 2.3. The method for receiving a cartridge by a diagnostic system of paragraph 1, wherein the Accession ID data is patient ID data.

Paragraph 3. A pathogen detection device comprising: a pathogen detector circuit configured to detect a target analyte in a patient sample, determine an identity of a pathogen from the target analyte, and generate a detection result including the identity of the pathogen; a cartridge receiving circuit configured to receive first scan bar code data; a scan data comparing circuit configured to compare first scan bar code data and expected scan bar code data; a user input circuit configured to receive a second scan bar code data from a user thereby reconciling first scan bar code data and expected scan bar code data.

Paragraph 4. A non-transitory computer readable storage medium comprising computer executable instructions, the computer executable instructions executable by a processor, the computer executable instructions comprising: instructions executable to receive first scan bar code data; and instructions executable to compare the first scan bar code data to expected scan bar code data; and instructions executable to display a field for user input, wherein received user input reconciles first scan bar code data to expected scan bar code data.

Paragraph 5. A pathogen detection device comprising: a pathogen detector circuit; a bar code scanner circuit that receives first bar code scan data and compares it to expected bar code scan data; a manual bar code input circuit to display a graphical user interface to accept user input of second bar code scan data; and a bar code reconciler circuit configured to compare first bar code data, second bar code data and expected bar code data.

Paragraph 6. A pathogen detection device comprising: a display device; and a processor configured to: receive bar code scan data and if the bar code scan data only comprises a first bar code scan data to display on the display device a field for user input of second bar code scan data.

Paragraph 7. The pathogen detection device of Paragraph 6, wherein the field for user input provides the field for manually entering second bar code scan data.

Paragraph 8. A pathogen detection device comprising: a display device; and a processor configured to: display, on the display device, a graphical user interface that provides a field for manual entry of bar code scan data; receive a user input of bar code scan data; apply the bar code scan data to expected bar code scan data read from a sample cartridge; and in response to the bar code scan data and expected bar code scan data being reconciled, accept the sample cartridge.

Paragraph 9. A pathogen detection device comprising: a display device; and a processor configured to: receive first bar code scan data from a sample cartridge; apply the first bar code scan data to expected bar code scan data read from a sample cartridge; display, on the display device, in response to the first bar code scan data and expected bar code scan data not being reconciled, a graphical user interface that provides a field for manual entry of second bar code scan data; receive a user input of bar code scan data; and in response to the second bar code scan data and expected bar code scan data being reconciled, accept the sample cartridge.

### Cleaning Card Workflow

Cleaning Cards are cards that are inserted into the analysis devices and are used to clean the analysis device. Historically, there was no system in place to ensure routine and consistent cleaning.

The bay will be automatically disabled when a cleaning is due. Further, bay monitoring systems will indicate when Cleaning Card must be discarded, which ensures cards aren't run more than a certain number of times or more than 24 hours between runs.

In some embodiments, a bay requires cleaning. A bay can be cleaned using a Cleaning Card.

### Initiate Cleaning

In some embodiments, a bay must be cleaned according to a particular frequency (Weekly, Monthly, or Custom). In some embodiments, the bay cleaning frequency is defined by the laboratory, and in some embodiments, the bay cleaning frequency is defined by the diagnostic instrument provider.

In some embodiments, if a bay cleaning is due, the bay is disabled until it has successfully processed a Cleaning Card. In some embodiments, if a bay is cleaned, the bay is reenabled.

In some embodiments, the GUI has a clean bay button, and the user taps the clean bay button and then selects the bay to be cleaned. In some embodiments, the user selects the bay icon on the GUI and the GUI then displays various details about the bay, such as the last run time, last Bay QC, whether the bay is reserved, and the last time the bay was cleaned. The bay details will also have a “clean the bay” button the user can tap. In some embodiments, any bay can be cleaned. In some embodiments, only bays that have been automatically disabled can be cleaned. In some embodiments, only bays scheduled for cleaning can be cleaned.

Once a user taps the clean bay button, the bay's LED light changes to indicate that the bay can accept the Cleaning Card. In some embodiments, only bays that have had their Clean Bay Button pressed can accept a Cleaning Card. In some embodiments, any bay can accept a Cleaning Card.

In some embodiments, to initiate cleaning, the user scans a bar code on the Cleaning Card. In some embodiments, to initiate cleaning, the user scans a bar code on the Cleaning Card, and the user can insert the Cleaning Card into any available bay. In some embodiments, to initiate cleaning, the user scans a bar code on the Cleaning Card, and the user can insert the Cleaning Card into any bay whose light has been changed to indicate it can accept a Cleaning Card.

In some embodiments, the Cleaning Card has a single bar code, wherein the single bar code identifies the Cleaning Card as a Cleaning Card. In some embodiments, the Cleaning Card has two bar codes, wherein both bar codes identify the Cleaning Card as a Cleaning Card.

In some embodiments, if the user taps the clean bay button, but then scans a cartridge that is not a Cleaning Card, the system stops the run. In some embodiments, if the user taps the clean bay button, but then scans a cartridge that is not a Cleaning Card, the system stops the run and then puts the bay into a ready-to-accept-cartridge state. The ready-to-accept-cartridge state means that the bay is able to accept any cartridge, Sample Cartridge, QC Cartridge, Bay OQ Card, or Cleaning Card.

In some embodiments, if the user taps the clean bay button, but then scans a cartridge that is not a Cleaning Card, the system logs the event as an incomplete Cleaning Card request.

In some embodiments, if the cleaning card was used less than 24 hours ago or has been used 6 times, the software displays an error (“The cleaning card was used less than 24 hours ago. Please wait for 24 hours before using it again.” or “The cleaning card has reached its maximum usage. Please discard it.”) and this stops the cleaning card run. If the cleaning card is an MAS cleaning card, these rules do not apply, thus this run continues.

The software then puts the bay in the ready-to-accept-cartridge state (by sending that command to the firmware) and blink the LED light white.

### Cleaning Card Run

In some embodiments, after the user inserts a Cleaning Card into the bay, the ISW commands the bay to raise pogo pins for 5 seconds. After the user inserts a Cleaning Card into the bay, the ISW commands the bay to raise pogo pins for 5 seconds and displays a progress bar for a total of 10 seconds in the bay tile. After 5 seconds, the ISW lowers the pogo pins. After the bay has been cleaned, the ISW logs the cleaning event.

### After Cleaning

In some embodiments, after the bay has been cleaned, the Cleaning Card is ejected from the bay. After the bay has been cleaned, the software saves cleaning run information such as username, date, timestamp, bay serial number, bay location, Cleaning Card serial number, Cleaning Card Accession ID.

In some embodiments, after the bay has been cleaned, the ISW performs a POST on the bay, then puts it in the ready state. In some embodiments, the Post comprises cleaning run information such as username, date, timestamp, bay serial number, bay location, Cleaning Card serial number, Cleaning Card Accession ID.

In some embodiments, bay monitoring systems indicate when a Cleaning Card must be discarded. In some embodiments, a Cleaning Card must be discarded if it has been used once, twice, three, four, five, six, seven, eight, nine, or ten times. In some embodiments, a Cleaning Card must be discarded if it was opened more than 24 hours before. In some embodiments, a Cleaning Card must be discarded if more than 24 hours pass between cleanings.

In some embodiments, when the user taps the View Details button, the system displays a cleaning card report for that cleaning run.

Cleaning a bay can be further understood by the following numbered paragraphs:

Paragraph 1. A method for cleaning a diagnostic instrument bay, the method comprising: evaluating by a diagnostic instrument bay monitoring system whether a bay cleaning is due; receiving by the detection instrument loading a Cleaning Card into a diagnostic instrument bay; and processing by the detection instrument the Cleaning Card by the diagnostic instrument bay.

Paragraph 2. A method for cleaning a diagnostic instrument bay, the method comprising: evaluating by a diagnostic instrument bay monitoring system whether a bay cleaning is due; disabling the diagnostic instrument bay by the detection instrument; receiving by the detection instrument a Cleaning Card into a diagnostic instrument bay; processing by the detection instrument the Cleaning Card by the diagnostic instrument bay; and enabling the diagnostic instrument bay to receive a Sample Cartridge, QC Cartridge, or bay QC Cartridge.

Paragraph 3. A method for cleaning a diagnostic instrument bay, the method comprising: evaluating by a diagnostic instrument bay monitoring system whether a bay cleaning is due; scanning by a user a Cleaning Card into a diagnostic instrument bay; evaluating by a diagnostic instrument bay monitoring system if the scanned Cleaning Card has been used more than the permitted times; and processing the Cleaning Card by the diagnostic instrument bay if the scanned Cleaning Card has been used less than the permitted times.

Paragraph 4. A method for cleaning a diagnostic instrument bay, the method comprising: evaluating by a diagnostic instrument bay monitoring system whether a bay cleaning is due; receiving by the detection instrument a Cleaning Card into a diagnostic instrument bay; evaluating by a diagnostic instrument bay monitoring system if the scanned Cleaning Card has been used more than the permitted times; and ejecting the Cleaning Card by the diagnostic instrument bay if the scanned Cleaning Card has been used more than the permitted times.

Paragraph 5. A method for cleaning a diagnostic instrument bay, the method comprising: evaluating by a diagnostic instrument bay monitoring system whether a bay cleaning is due; receiving by the detection instrument a Cleaning Card into a diagnostic instrument bay; evaluating by a diagnostic instrument bay monitoring system if the scanned Cleaning Card is available for use wherein a Cleaning Cardis available for use if the last time it has been used was less than 24 hours before; and ejecting the Cleaning Card by the diagnostic instrument bay if the scanned Cleaning Card is not available for use or processing the Cleaning Card if the scanned Cleaning Card is available for use.

### Example 1: Editing Control Alerts of an Assay

### Change the Rotation Order

The control order was originally A, B, and C and the “Days Until Due” for A, B, and C were 30, 60, and 90 respectively. The order is changed to B, A, and C. After changing the order, the “Days Until Due” for A, B, and C are now 60, 30, and 90 days, respectively.

An external control's Days Until Due value is recalculated based on its new order.

### Change Run Frequency to Weekly, Monthly, or Custom

The control frequency was A weekly, B weekly, and C monthly, and the “Days Until Due” for A, B, and C were 7, 7, and 30 respectively. The frequency is changed to A weekly, B weekly, and C weekly. After changing the frequency, “Days Until Due” for A, B, and C are now 7, 7, and 7 respectively.

An external control's Days Until Due value is recalculated based on the new frequency.

Change Run Frequency from Weekly, Monthly, or Custom to Off

The external controls of that assay become standalone (Run Frequency is off). The control frequency was A weekly, B weekly, and C monthly, and the “Days Until Due” for A, B, and C were 7, 7, and 30 respectively. The frequency is changed to “off” After changing the frequency, “Days Until Due” for A, B, and C are now 0, 0, and 0 respectively.

The Days Until Due value is set to nothing, i.e., zero.

Change Rotation from on to Off

When the rotation is turned off, the external controls of that assay become standalone (Rotation is off).

The control frequency was A weekly, B weekly, and C monthly and rotation was B, A then C and the “Days Until Due” for A, B, and C are 7, 7, and 30 respectively. If the rotation is turned off, the “Days Until Due” for A, B, and C are now, 7, 7, and 30 respectively.

The system recalculates the Days Until Due value based on the Run Frequency value.

### Example 2: Rotation Order is ON and Frequency is ON and a Successful Control Run has been Completed

The Days Until Due recalculates for all subsequent controls based on the actual run date of the completed control.

Rotating controls A, B, and C in that order with a frequency of 30 days. After creation, “Days Until Due” for A, B, and C are now, 30, and 60, respectively. After A is run, “Days Until Due” for A, B, and C become 90, 30, and 60, respectively. After 10 days have passed, “Days Until Due” for A, B, and C are 80, 20, and 50, respectively. If B is run now, “Days Until Due” for A, B, and C become 60, 90, and 30, respectively.

TABLE 1
Update the Days Until Due after successful control run

| | Original Value | | Updated value |
|---|---|---|---|
| Panel | A, B, C | Successful control | A, B, C |
| Rotation | A = 1 | run for control | A = 3 |
| Order | B = 2 | panel A | B = 1 |
| | C = 3 | | C = 2 |
| Days Until | A = 30 | | A = 90 |
| Due | B = 60 | | B = 30 |
| | C = 90 | | C = 60 |

### Example 3: Rotation Order is ON and Frequency is ON and the Days Until Due is Modified when the Run Frequency is Changed

The Days Until Due is modified when the run frequency is changed.

TABLE 2
Modified Days Until Due When The Rotation Order Is Changed

| | Original Value | Updated value |
|---|---|---|
| Panel | A, B, C | A, B, C |
| Rotation Order | A = 1 | A = 3 |
| | B = 2 | B = 1 |
| | C = 3 | C = 2 |
| Days Until Due | A = 30 | A = 90 |
| | B = 60 | B = 30 |
| | C = 90 | C = 60 |

### Example 4: Rotation Order is ON and Frequency is ON and a Successful Control Run has been Completed Early

If a control is run prior to its actual due date, the due dates of its successors will be moved up to reflect the new logical order of the controls within the existing sequence as well as for subsequent control sequences. The initial intent of the customer based on the frequency input should not be broken. e.g. if a control is expected to run every month, then a month cannot be skipped simply because a control run was completed in sequence but too early. In some embodiments, once a sequence is complete, the original order may be resumed as expected. In some embodiments, once a sequence is complete, the modified order continues as expected.

TABLE 3
Update to Days Until Due after successful early control run

| | Original Value | | Updated value |
|---|---|---|---|
| Panel | A, B, C | Successful control | A, B, C |
| Rotation | A = 1 | run for control | A = 3 |
| Order | B = 2 | panel A | B = 1 |
| | C = 3 | at day 10 | C = 2 |
| Days Until | A = 30 | | A = 90 |
| Due | B = 60 | | B = 30 |
| | C = 90 | | C = 60 |
| | A = 120 | original | Modified |
| | B = 150 | order | order |
| | C = 180 | resumed | maintained |
| | | A = 120 | A = 180 |
| | | B = 150 | B = 120 |
| | | C = 180 | C = 150 |

TABLE 4
Update to Days Until Due after successful early control run

| | Original Value | | Updated value |
|---|---|---|---|
| Panel | A, B, C | Successful | A, B, C |
| Rotation | A = 1 | control run | A = 3 |
| Order | B = 2 | for control | B = 1 |
| | C = 3 | panel A on | C = 2 |
| Frequency | The first of every | January 20 | The first of every |
| | month | | month |
| Days Until | A = February 1 | | A = April 1 |
| Due | B = March 1 | | B = February 1 |
| | C = April 1 | | C = March 1 |

| | |
|---|---|
| original | Modified |
| order | order |
| resumed | maintained |
| A = May 1 | A = July 1 |
| B = June 1 | B = May 1 |
| C = July 1 | C = June 1 |

### Example 5: Rotation Order is ON and Frequency is ON and a Successful Control Run has been Completed but Out of Order

In some embodiments, the system allows users to run controls out of order. When a control is run out of order, the system should then expect the next control in sequence based on the last successful control run. Once a sequence is complete, the original order may be resumed as expected.

TABLE 5
Update to Days Until Due after successful control run out of order

| | Original Value | | Updated value |
|---|---|---|---|
| Panel | A, B, C | Successful control | A, B, C |
| Rotation | A = 1 | run for control | A = 2 |
| Order | B = 2 | panel B | B = 3 |
| | C = 3 | | C = 1 |
| Days Until | A = 30 | | A = 60 |
| Due | B = 60 | | B = 90 |
| | C = 90 | | C = 30 |

Rotating controls A, B, and C in that order with a frequency of 1st Monday of every 1 Month. After creation, “Days Until Due” for A, B, and C are Now, 30, 60 and 90 respectively. After A is run, “Days Until Due” for A, B, and C become 90, 30, and 60 respectively. After 10 days have passed, “Days Until Due” for A, B, and C are 80, 20, and 50 respectively. If B is run now, “Days Until Due” for A, B, and C become 50, 80, and 20 respectively, where control C is to be due next on the first Monday of the next month (which is 20 days away in this example). Control A becomes 50 days, which is the first Monday of the month that follows C (and is 30 days after C in this example).

### USE CASES—QC Workflow

The system can be further understood by the following preconditions and post conditions:

TABLE 6

| No. | Preconditions | Postconditions |
|---|---|---|
| 1 | There are no assays installed in the system | The screen displays “There are no assays |
| | User navigates to QC Scheduler | installed.” |
| 2 | There are some assays installed | The screen displays “No QC has been created” |
| | There are no external controls defined | The screen displays the Create New QC button |
| | User navigates to QC Scheduler | |
| 3 | There are some assays installed | The system displays the screen with the list of |
| | There are some external controls defined | QC Controls |
| | User navigates to QC Scheduler | The system displays the assay drop-down for |
| | | all installed assays that have external control |
| | | rules set established |
| | | When the user selects a particular assay in the |
| | | drop-down, the list of QC Controls updates to |
| | | list only controls for the selected assay |
| | | System displays the Manage QC Rules and the |
| | | Create New QC buttons |
| 4 | There are some assays installed | The Create New QC page is displayed, it |
| | User clicks Create New QC button | contains text controls to specify Accession ID |
| | | and comments, assay drop-down listing |
| | | installed assays, and Create button (initially |
| | | disabled) |
| | | When the user selects an assay from the drop- |
| | | down, the grid to specify Detected/Not |
| | | Detected result for each assay target is |
| | | displayed |
| | | After the user specifies the Accession ID for |
| | | the selected assay, the Create button is enabled |
| 5 | The user entered information in the Create | The new QC Controls appear in the list of QC |
| | New QC page and clicked Create | Controls on the scheduler page |
| | The user defined a QC Control for an assay, | Target 2 is removed from the QC Control |
| | then enabled/disabled some targets for the | definition automatically since it was disabled |
| | assay and ran the QC Control | from the assay resulting in the QC Control with |
| | For example: The assay has target 1, target | Target 1 only. |
| | 2, and target 3 | At that time, the assay contains Target 1 and |
| | User disables Target 3 and defines QC | Target 3 while the QC contains Target 1. |
| | Control (which includes Target 1 and Target | |
| | 2) | |
| | Then user enables Target 3 and disables | |
| | Target 2 and runs the QC Control | |
| 6 | There are some assays installed | The Manage QC Rules window is displayed |
| | There are some QC Controls defined | It contains boxes for each installed assay type |
| | User navigates to QC Scheduler and clicks | listing Frequency, Changing Lot, Rotation |
| | Manage QC Rules button | parameters, and a button to edit those |
| | | parameters. The button text is Add when the |
| | | frequency is OFF and Edit otherwise. |
| 7 | User clicks on the Add/Edit button for | The Assay Control Alerts dialog page is |
| | particular assay at the Manage QC Rules | displayed. |
| | window | It contains the list of controls defined for this |
| | | assay, and controls to choose Run frequency, |
| | | changing Lot Alert, Require Valid Controls to |
| | | perform tests. |
| | | For the admin, internal, service users “Reset |
| | | due dates” button is available |
| 8 | In the Assay Control Alerts dialog, the user | The Rotation option becomes visible |
| | changes the Run Frequency from Off to a | The QC Controls in the list update to display |
| | different value | Days Until Due parameter |
| 9 | In the Assay Control Alerts dialog, the user | For each QC Control in the grid, the rotation |
| | enables Rotation | order control appears, and the Days Until Due |
| | | parameter is cleared. By default, the rotation |
| | | order is not assigned, and the available options |
| | | for rotation order are numbers in the range 1 to |
| | | total number of controls and “All”. As user |
| | | assigns the rotation order, the “Days Until |
| | | Due” parameter is updated |
| | | If the user doesn't assign valid rotation order |
| | | for some controls, “Days Until Due” parameter |
| | | for them is not listed and frequency for those |
| | | controls is not enforced. |
| | | A user cannot save controls if a logical order |
| | | has not been met. |
| | | A user cannot save controls if rotation is ON |
| | | and an order is not assigned to some controls. |
| | | The Save button is greyed out until all logical |
| | | requirements are met. |
| | | If controls are assigned an order, but the order |
| | | is not logical, and a user attempts to Save, a |
| | | dialogue appears instructing them “A logical |
| | | condition for defined controls has not been |
| | | met. Please evaluate defined control |
| | | parameters.” |
| 10 | In the Assay Control Alerts screen, the user | The specified alert parameters for an assay get |
| | clicks Apply Alerts | saved, both Assay Control and Manage QC |
| | | Rules windows are closed |
| | | The list of controls in the grid under QC |
| | | Scheduler is updated to reflect correct |
| | | frequency, rotation order, and Days Until Due |
| | | parameters |
| 11 | In the Assay Control Alerts screen, the user | If no changes were made, The Assay control |
| | clicks Cancel | Alerts screen closes, and the user returns to the |
| | | previous screen. The changes made by user are |
| | | not saved, with the exception of changes that |
| | | happened if the user clicked “Reset Due Dates” |
| | | If changes were made, then a user clicks |
| | | cancel, a confirmation dialogue appears stating |
| | | “Are you sure you want to cancel? All unsaved |
| | | changes will be deleted.” |
| 12 | In the Assay Control Alert screen the user | The dialog box is displayed asking for |
| | clicks Reset due dates | confirmation |
| | | If the user confirms, the due dates for controls |
| | | belonging to this assay get reset and the Days |
| | | Until Due parameter in the grid is updated |
| 13 | In the Assay Control Alert screen the user | The due dates of controls in the list become |
| | changes run frequency (for example, number | updated |
| | of days, monthly to weekly etc.) | |
| 14 | In the Manage QC Rules screen the user | The changes get saved |
| | clicks the Apply Changes button | |
| 15 | In the QC Scheduler screen, the user selects | The details for the selected control, including |
| | one of the controls in the list | QC barcode, expected Detected and Not |
| | | detected targets get displayed |
| | | Buttons to export and print the QC Control |
| | | reports are available |
| | | Combobox Action and Run QC button are |
| | | available |
| | | If the control has not been run yet, Edit button |
| | | is available |
| 16 | In the QC Scheduler screen, the user selects | The edit QC Control windows is displayed, |
| | the ellipses for a single control in the list | similar to Create new QC window |
| | The control has not been run previously | The Accession ID, expected target results and |
| | The user clicks Edit Control button from the | comments can be changed |
| | droplist | The assay can't be changed |
| 17 | In the QC Scheduler screen, the user selects | The edit QC Control windows is displayed, |
| | the ellipses for a single control in the list | similar to Create new QC window |
| | The control has been run previously | The expected target results and comments can |
| | The user clicks Edit Control button from | be changed |
| | droplist | The assay and Accession ID can't be changed |
| 18 | In the QC Scheduler screen, the user selects | The system asks for confirmation |
| | one of the controls in the list | If the user confirms, the control is deleted |
| | The user selects Delete from the Action | |
| | menu | |
| 19 | The user defines a QC Control for assay, and | The diagnostic system mark the control as |
| | deactivates the assay | invalid (with the words PLEASE DELETE) |
| 20 | The user defines a QC Control for assay, and | The diagnostic system mark the control as |
| | activates the assay of the same type with | invalid (with the words PLEASE DELETE) |
| | different targets | |
| 21 | A user defines QC Frequency using the | Days Until Due recalculates based on the |
| | “Custom” tab and then uses the Days | frequency setting after completion of QC |
| | function for the frequency. | sequence or event. |
| | Rotation is either on or off | |
| | A user runs a QC (either the full set or a | |
| | single sequence) before the QC due date | |
| 22 | The user clicks on the Run button in the QC | If there are no bays available, the message “No |
| | Scheduler screen | bays are available” pops up |
| | | If there are available bays, the system prompts |
| | | the user to scan the cartridge |
| | | After the cartridge is scanned, if the |
| | | information matches the defined QC Control, |
| | | the prompt is displayed to insert the cartridge |
| | | into an available bay |
| | | If the scanned cartridge doesn't match the |
| | | defined external control (wrong accession, |
| | | wrong assay, wrong targets), the message pops |
| | | up indicating the problem and asking the user |
| | | if they want to continue. If the user answers |
| | | Yes, the data from the scanned barcode is |
| | | overrides the QC Control selection. |
| | | If the user doesn't insert the cartridge, the |
| | | timeout message pops up |
| | | After the user inserts the cartridge, the dialog |
| | | pops up. It allows to edit product (material) lot |
| | | number and comments, and has “Done” button |
| | | After dialog is dismissed, it can be invoked |
| | | again by clicking again on the “Run QC” |
| | | button, if the test is still in the running state |
| | | The column “Status” is updated with the run |
| | | status while the preflight is processing and |
| | | indicates in color if it fails. After the cartridge |
| | | is removed the column status returns to |
| | | indicate number of days/due/overdue status |
| 23 | The user scans the barcode, where the | Based on protocol ID, the systems checks |
| | Accession ID and assay type match the | assay targets. If assay targets are the same as |
| | external control | the ones defined in the control, the run is |
| | | recognized as QC Control run. Otherwise it's |
| | | treated as sample run. |
| 24 | After starting the run from QC Controls | The dialog “Test starting” is displayed |
| | screen, the user inserts the cartridge, and | It contains some indication that the preflight is |
| | preflight begins | in process |
| | | The “Status” column is updated with the |
| | | information that the preflight is in process |
| | | The “run date” column is updated |
| | | It contains Product Lot Number: Add button. |
| 25 | After starting the run from QC Controls | The dialog “Test starting” updates to “Test |
| | screen, the preflight completes and the run | started |
| | starts | The “Status” column is updated to “Running” |
| 26 | The user scans cartridge, and the cartridge is | If the Require Valid Control to Perform Test |
| | identified as sample run | setting is enabled, the user is prevented from |
| | There are QC Controls for the same assay | starting the run. Otherwise, a warning is |
| | that are due | generated |
| 27 | The user scans a cartridge, and the cartridge | The system verifies that each QC Control has |
| | is identified as sample run | been run successfully in the last cycle with the |
| | There are QC Controls for this assay that | cartridge belonging to the same lot as the |
| | have been run and are not due yet | current cartridge. If not, depending on Run |
| | Changing Lot Alert setting is enabled | Frequency setting, the system prevents the run |
| | | or generates a warning. |
| 28 | The user selects a QC Control and then | The system switches to a Run History view, |
| | selects Run History in the display | and displays the history of the QC Control: last |
| | | run, result, etc. |
| 29 | The user selects a QC Control and then | The dialog appears, allowing to select the type |
| | selects Export in the dialog box | of the report and path |
| | | The report is exported |
| 30 | The user selects a QC Control and then | The barcode is sent to printer or email, text |
| | selects either Print barcode or send barcode | message, instant message, etc. |
| 31 | QC notification popup | The notification is displayed each time the user |
| | | logs in when QC Controls are due |
| 32 | Rotation and Frequency is ON and Custom | The Days Until Due for the next control |
| | is selected with [x] number of days between | updates to pull in the defined number of days |
| | controls | from the date of the most recent completed |
| | A control is run prior to its actual due date | control |
| | The external control run completed | |
| | successfully | |
| 33 | Rotation and Frequency are ON with | The Days Until Due for the next control is |
| | Frequency set to Monthly and the First | pulled it to match the expected due date of the |
| | Monday of every 1 Month | control that had just completed |
| | A control is run prior to its actual due date | E.g. If Control B is scheduled to run on March |
| | (middle of the month) | 1, and Control A of the next sequence is |
| | The external control run completed | scheduled to run on April 1, but Control B is |
| | successfully | instead run on February 15^{th}, Control A of the |
| | | next sequence now becomes due on March 1, |
| | | and Control B of the next sequence, having just |
| | | been run, is now due April 1. This satisfies the |
| | | customers need to ensure that QC is run every |
| | | month, and just because a QC is run early, a |
| | | month is not skipped. |

TABLE 7
USE Cases- Run Workflow

| No. | Preconditions | Postconditions |
|---|---|---|
| 1 | User logs in with barcode or manually | Load/Display all bays without any status |
| | System configured with towers/bays | |
| | All bays are idle and available to start a run | |
| 2 | User logs in with barcode or manually | All available Bay LED lights blinking a color |
| | System configured with towers/bays | to prompt the user to insert a cartridge |
| | All bays or any bay is idle and available to | |
| | start a run | |
| | Assays are imported (ex: RP2) | |
| | Scan a cartridge with 2D barcode (RP2) and | |
| | Accession ID to start a run | |
| 3 | User logs in with barcode or manually | Specific (A1) Bay LED lights blinking a color |
| | System configured with towers/bays | to prompt the user to insert a cartridge into that |
| | Al bay is idle and available to start a run | bay |
| | Assays are imported (ex: RP2) | |
| | Select a specific bay (A1) by tapping the | |
| | bay icon on the GUI | |
| | Scan a cartridge with 2D barcode (RP2) and | |
| | Accession ID to start a run | |
| 4 | After running Use case 2 or 3 above | Initialization of the bay fails and displays DNS |
| | Cover the PCB electrodes with the tape so | bay tile. |
| | that it fails on preflight | Bay LED light changes to a color to indicate |
| | User inserts the cartridge (any bay or A1) | the bay has failed |
| | | Tapping Bay tile (GUI icon), displays Test |
| | | Detail View for DNS |
| 5 | After running Use case 2 or 3 above | Initialization of the bay passes and displays |
| | User inserts the cartridge (any bay or A1) | progress bar bay tile. |
| | Run starts successfully | Bay LED light changes to a color indicating |
| | | the bay has passed initialization |
| 6 | After running Use case 5 above | Displays Test Detail View |
| | Tap or touch on running bay (A1) | Test Details view contains: |
| | | Accession ID (Editable if manually entered) |
| | | Patient ID (Add or Edit) |
| | | Cartridge ID/Lot/Expiration |
| | | Report Comments |
| 7 | After running Use case 5 above | Aborts the run and displays DNF on the bay |
| | Introduce Bay error by changing the Bay | tile. |
| | heater limits on the OPUS (ADF) | The Bay LED light changes to a color to |
| | | indicate the DNF |
| | | Tapping the Bay tile, displays Test Detail |
| | | View for DNF |
| 8 | After running Use case 5 above | Completes the run and displays Invalid Test |
| | Introduce AAM error by changing the DSP | Bay tile. |
| | or invalid pads | The Bay LED light changes to a color |
| | | indicating the Invalid bay |
| | | Tapping Bay tile, displays Test Detail View for |
| | | Invalid Test |
| 9 | After running Use case 5 above | Aborts the run and displays DNF bay tile. |
| | User aborts the run in Test Details view | The Bay LED light changes to a color to |
| | | indicate the DNF |
| 10 | After running Use case 5 above | Completes the run and displays Test complete |
| | Make sure the test is completed | bay tile. |
| | successfully without any errors and zero or | The Bay LED light changes to a color |
| | more targets detected | indicating the test is complete |
| | | Tapping Bay tile, displays Test Detail View for |
| | | Test Complete |
| 11 | User logs in with barcode or manually | Display QC Notification dialog box |
| | System configured with towers/bays | |
| | Assays are imported (ex: RP2) | |
| | QC Controls are created/configured, and | |
| | some controls are due | |
| 12 | User logs in with barcode or manually | Run the accession as PTO |
| | System configured with towers/bays | |
| | All bays or any is idle and available to start | |
| | a run | |
| | Assays are imported (ex: RP2) | |
| | PTO orders are imported from LIS | |
| | Match the cartridge accession with PTO | |
| | Order | |
| | Scan a cartridge with 2D barcode (RP2) and | |
| | accession to start a run | |
| 13 | User logs in with barcode or manually | Run the accession as QC/External Control run. |
| | System configured with towers/bays | Complete the run displaying bay tile Test |
| | All or any bay is idle and available to start | complete “QC Passed/Failed” |
| | a run | |
| | Assays are imported (ex: RP2) | |
| | QC Controls are created/configured | |
| | Match the accession with QC Control | |
| | Scan a cartridge with 2D barcode (RP2) and | |
| | accession to start a run | |
| 14 | User logs in with barcode or manually | Display Manual entry dialog for the manual |
| | System configured with towers/bays | entry of the accession ID |
| | All or any bays is idle and available to start | |
| | a run | |
| | Assays are imported (ex: RP2) | |
| | Scan a cartridge with 2D barcode (RP2) and | |
| | without accession barcode to start a run | |
| 15 | User logs in with barcode or manually | Load/Display all bays on home screen and |
| | System configured with towers/bays | displays Bay Disabled Bay tile for the disabled |
| | Manually Disable one of the bays (A1) | bay (A1) |
| 16 | User logs in with barcode or manually | Load/Display all bays on home screen and |
| | System configured with towers/bays | displays Bay Auto Disabled Bay tile for the |
| | Auto Disables one of the bays (A1) | disabled bay (A1) |
| 17 | User logs in with barcode or manually | Display Bay Settings screen (A1) dialog box |
| | System configured with towers/bays | Display/input/Error - Reason to disable the bay |
| | Manually Disables one of the bays (A1) | Show all bay stats including the last service |
| | Select a specific bay (A1) by tapping | date |
| | Settings icon right top corner | |
| 18 | User logs in with barcode or manually | Display Bay Settings screen (A1) dialog box |
| | System configured with towers/bays | Show all bay stats including the last service |
| | Load/Display all bays on home screen | date |
| | Select a specific bay (A1) by tapping | |
| | Settings icon right top corner | |
| 19 | User initiates the cleaning workflow for a | System prompts User to scan a Cleaning Card |
| | bay | System processes the barcodes |
| | User scans a Cleaning Card | System puts the bay in ready-to-accept- |
| | User inserts the Cleaning Card | cartridge state |
| | | System does the “cleaning” (by raising the |
| | | pogo pins for xx seconds, then lowering them) |
| | | System ejects the Cleaning Card |
| | | The cleaning run info is recorded in the |
| | | database |
| 20 | User initiates the cleaning workflow for a | System prompts User to scan a Cleaning Card |
| | bay | System displays an error: “The scanned |
| | User scans a cartridge | cartridge is not a Cleaning Card.” |
| 21 | User marks 3 bays (e.g., A1 A2 A3) in the | The 3 bays are marked with “Perform OQ” in |
| | Bay Configuration screen to perform OQ | the Run View |
| 22 | User taps an OQ-marked bay (e.g., A1) to | System prompts User to scan an OQ card |
| | start an OQ run | System processes the barcodes |
| | User scans an OQ card | System puts the bay in ready-to-accept- |
| | User inserts the OQ card | cartridge state |
| | | System runs OQ |
| | | System ejects the card |
| | | System records OQ results in the database |
| 23 | User taps an OQ-marked bay (e.g., A1) to | System prompts User to scan an OQ card |
| | start an OQ run | System processes the barcodes |
| | User scans a Sample Cartridge | System displays an error: “The scanned |
| | | cartridge is not an OQ card.” |

External controls can be further understood by the following numbered paragraphs:

Paragraph 1. A pathogen detection device comprising: a quality control (QC) rule database configured to store QC rules; and a QC customization module communicatively coupled to the QC rule database.

Paragraph 2. The pathogen detection device of claim 1, wherein a first QC rule stored in the QC rule database comprises a first QC target having a first days until due deadline.

Paragraph 3. The pathogen detection device of paragraph 2, wherein a second QC rule stored in the QC rule database comprises a second QC target having a second days until due deadline.

Paragraph 4. The pathogen detection device of paragraph 2, wherein if the first QC target is changed in the QC customization module, the first QC rule stored in the QC rule database is updated.

Paragraph 5. The pathogen detection device of paragraph 2, wherein if the first QC target is changed in the QC customization module, a second QC Rule stored in the QC rule database is updated.

Paragraph 6. The pathogen detection device of paragraph 2, wherein a QC alert module sends a notification to a user interface when the first days until due deadline is between 0 and 10 days.

Paragraph 7. The pathogen detection device of paragraph 2, wherein a QC violation module sends a notification to a user interface when the first days until due deadline is less than zero.

Paragraph 8. The pathogen detection device of paragraph 2, wherein after a QC test is processed a QC pass/fail module sends a notification to a user interface that the QC test past quality control assessment if the QC test ran successfully to completion.

Paragraph 9. The pathogen detection device of paragraph 2, wherein after a QC test is processed a QC report module sends a report to a user interface indicating the result of the QC test that was processed.

Paragraph 10. A pathogen detection device comprising: a QC rule database configured to store QC rules and communicatively coupled to a installed assay database; and the installed assay database comprising a first installed assay having at least a first target.

Paragraph 11. The pathogen detection device of paragraph 10, wherein the QC rule database is empty prior to input from a user.

Paragraph 12. The pathogen detection device of paragraph 10, wherein the pathogen detection device does not comprise a second uninstalled assay and wherein the QC rule database is configured to store QC rules for a first installed assay but not the second uninstalled assay.

Paragraph 13. The pathogen detection device of paragraph 10, wherein the QC rule comprises a QC Accession ID and wherein the QC Accession ID is correlated to a QC cartridge Accession ID prior to processing a QC cartridge.

Paragraph 14. The pathogen detection device of paragraph 10, wherein the first installed assay further comprises a second target and wherein the QC rule database is configured to store a first QC rule for the first target and a second different QC rule for the second target.

Paragraph 15. The pathogen detection device of paragraph 10, wherein the first installed assay further comprises a second target and wherein the QC rule database is configured to store QC rules for the first target and the second target but the QC rule for the second target is disabled.

Paragraph 16. A system for managing quality control for a pathogen detection device comprising: a quality control (QC) rule database configured to store QC rules on the pathogen detection device; a QC customization module configured to allow a user to change QC rules stored on the pathogen detection device; and a user interface configured to allow a user to access the QC customization module.

Paragraph 17. The system for managing quality control for a pathogen detection device of paragraph 16, wherein the QC customization module is configured to receive QC rule inputs from a user wherein the QC rule inputs include QC rule run frequency, QC rule rotation, whether lot alerts are activated, or whether the system must have a valid control before processing a sample.

Paragraph 18. The system for managing quality control for a pathogen detection device of paragraph 16, wherein the QC customization module is configured to generate a modification to a second QC rule when a first QC rule is changed.

Paragraph 19. The system for managing quality control for a pathogen detection device of paragraph 16, wherein the QC customization module is configured to block modifications to a first QC rule when a modification to the first rule does not have logical consistency with a second QC rule.

Paragraph 20. The system for managing quality control for a pathogen detection device of paragraph 16, further comprising a QC alert module wherein each QC rule has an expected result and when a QC test does not generate a result that matches the expected result the QC alert module sends a notification to the user interface.

### Dynamic Panel Ordering

### SUMMARY

A system, a method, and a user interface are described for customizing reporting results for a diagnostic system. Each diagnostic assay panel is capable of detecting a panel of targets. Hospitals can define which targets from an assay panel they want included in a Detection Report. The cartridge detects every target on the assay panel, but only the selected targets are reported to the hospital/doctor in a Detection Report. As a result, some Detection Results are not included in a Detection Report. This means, in some instances, a detected target is not included in a Detection Report.

### BACKGROUND OF THE INVENTION

Historically, every target on an assay panel is evaluated, and the results of every target are reported to the hospital/doctor. In some cases, this is too much information; the hospital/doctor only wants a subset of relevant information.

### SUMMARY OF THE INVENTION

Hospitals can now define which targets from an assay panel they want reported. The diagnostic instrument still evaluates every target on the assay panel, but only the selected targets are reported to the hospital/doctor. In this way, known Detection Results are withheld from the Detection Report and hospital/doctor.

### DETAILED DESCRIPTION

### Installing an ADF

When a laboratory receives a new diagnostic instrument or a new assay panel, they need to install a new ADF file.

In some embodiments, the user interface has a menu for ADF Installation. In some embodiments, only a user with appropriate credentials, such as a supervisor or administrator, can install a new ADF. In some embodiments, any user can install a new ADF.

### ADF Characteristics

In some embodiments, the ADF will contain configurable values (e.g. On/Off) for Dynamic Panel Ordering. In some embodiments, the ADF will contain configurable values (e.g. On/Off) in order to generate a new Dynamic Panel Rule.

In some embodiments, all ADF files installed on the detection instrument are capable of Dynamic Panel Ordering. In some embodiments, a first ADF file installed on the detection instrument is capable of Dynamic Panel Ordering and a second ADF file installed on the detection instrument is not capable of Dynamic Panel Ordering. In some embodiments, a first plurality of ADF files installed on the detection instrument are capable of Dynamic Panel Ordering and a second plurality of ADF files installed on the detection instrument are not capable of Dynamic Panel Ordering. In some embodiments, the Gastrointestinal panel ADF file is capable of Dynamic Panel Ordering and the respiratory, gram-negative, gram-positive and fungal assay protocol ADF files are not capable of Dynamic Panel Ordering. In some embodiments, the Gastrointestinal, respiratory, gram-negative, gram-positive and fungal assay protocol ADF files are capable of Dynamic Panel Ordering.

In some embodiments, all installed assay protocols on the detection instrument are capable of Dynamic Panel Ordering. In some embodiments, a first installed assay protocol on the detection instrument is capable of Dynamic Panel Ordering and a second installed assay protocol on the detection instrument is not capable of Dynamic Panel Ordering. In some embodiments, a first plurality of installed assay protocols on the detection instrument are capable of Dynamic Panel Ordering and a second plurality of installed assay protocols on the detection instrument are not capable of Dynamic Panel Ordering. In some embodiments, the Gastrointestinal assay protocol is capable of Dynamic Panel Ordering and the respiratory, gram-negative, gram-positive and fungal assay protocols are not capable of Dynamic Panel Ordering. In some embodiments, the Gastrointestinal, respiratory, gram-negative, gram-positive and fungal assay protocols are capable of Dynamic Panel Ordering

### Installing an ADF

After navigating in system settings in the GUI where the available panel assays are displayed, the user selects Import New Assay. The user installs an ADF with Dynamic Panel Ordering capability/architecture enabled. In some cases, even though the ADF has the ability to use Dynamic Panel Ordering, the installer can choose to turn Dynamic Panel Ordering off.

In some embodiments, installing a new ADF is accomplished by uploading a file that has an ADF with Dynamic Panel Ordering enabled from a thumb drive, from a CD, from a floppy disc, or by connecting to a cloud and downloading a file from the cloud. A skilled artisan knows how to install ADF files on a detection instrument. The system adds a new assay tile to the GUI for the newly added ADF for a new assay panel.

In some embodiments, when a new ADF is installed with Dynamic Panel Ordering enabled, all of the Detection Results for each target on the assay panel are reported in the Detection Report. The ADF 604 may comprise two portions including an OPUS file and an AAM file. In some embodiments, the AAM file defines whether Dynamic Panel Ordering is enabled and defines the targets on the assay panel that are reported in the Detection Report. In some embodiments, the AAM defines whether Dynamic Panel Ordering is enabled and defines the targets on the assay panel that are not reported in the Detection Report. In some embodiments, the AAM defines whether Dynamic Panel Ordering is enabled and defines the targets on the assay panel that are not capable of being deselecting, i.e., targets that are not configurable. In some embodiments, the AAM defines whether Dynamic Panel Ordering is enabled, and defines the targets on the assay panel that are reported in the Detection Report and defines the targets on the assay panel that are not reported in the Detection Report, and/or defines the targets on the assay panel that are not capable of being deselecting, i.e., not configurable.

### Activating an ADF for Dynamic Panel Ordering

In some embodiments, Dynamic Panel Ordering can be retroactively turned on for ADF files previously installed on a detection instrument. In some embodiments, Dynamic Panel Ordering cannot be retroactively turned on for ADF files previously installed on a detection instrument.

Deactivating Dynamic Panel Ordering from an ADF

In some embodiments, if an ADF is deactivated all Dynamic Panel Rules associated with the ADF are deactivated. In some embodiments, if an assay panel is deactivated all Dynamic Panel Rules associated with the assay panel are deactivated.

In some embodiments, if no Dynamic Panel Rule is active (a.k.a. enabled), the ADF becomes inactive on the system. In some embodiments, if no Dynamic Panel Rule is active (due to a user deactivating all Dynamic Panel Rules within an assay protocol or disabling all targets within each Dynamic Panel Rule), the ADF becomes inactive.

### New ADF Protocols

In some embodiments, when a new version of an ADF is installed, any Dynamic Panel Rules from the old ADF version will transfer over to the new ADF. In some embodiments, when a new version of an ADF is installed, any Dynamic Panel Rules, including but not limited to existing target configurations from the old ADF version, will transfer over to the new ADF. In some embodiments, when a new version of an ADF is installed, any Dynamic Panel Rules, including but not limited to Dynamic Panel Rule status (enabled or disabled), will be applied to the new ADF.

Sometimes, a new ADF file for an assay protocol is uploaded to the detection instrument that has additional targets than a previously loaded assay protocol. In some embodiments, when a new assay protocol/ADF is uploaded that has additional targets, all of the new targets are automatically added (selected for inclusion in a Detection Report) for each Dynamic Pane Rule already on the system. In some embodiments, when a new assay protocol is uploaded that has additional targets, all of the new targets are not added (not selected for inclusion in a Detection Report) for each Dynamic Pane Rule already on the system. In some embodiments, when a new assay protocol is added that has additional targets, a first new target is automatically added (selected) for each Dynamic Pane Rule already on the system, and a second new target is not automatically added (deselected) for each Dynamic Pane Rule already on the system. In some embodiments, if a new assay protocol includes new targets, the new target(s) become visible on all Dynamic Panels and are defaulted to the selected state. In some embodiments, if a new assay protocol includes new targets, the new target(s) become visible on all Dynamic Panels and are defaulted to the deselected state.

Sometimes, a new ADF file for an assay protocol is uploaded to the detection instrument that has fewer targets than a previously loaded assay protocol. In some embodiments, when a new assay protocol is added that has fewer targets, all of the deleted targets are automatically deselected for each Dynamic Panel Rule already on the system.

In some embodiments, if targets are modified (added or deleted) on an assay panel, when the new ADF is uploaded, the user is prompted to review Dynamic Panel Rules with the modified target to configure changes to the Dynamic Panel Rules, if any.

ADF files can be further understood by the following numbered paragraphs:

Paragraph 1. An assay definition file configured to enable a detection instrument to receive a user selection of a first target for inclusion in a Detection Report.

Paragraph 2. An assay definition file configured to enable a detection instrument to receive a user selection of a first target for exclusion from a Detection Report.

Paragraph 3. An assay definition file configured to enable a detection instrument to receive a user selection of a first target for inclusion in a Detection Report and a second target for exclusion from the Detection Report.

Paragraph 4. An assay definition file configured to enable a detection instrument to receive a user selection of a first target for inclusion in a Detection Report and a second target, which is not configured for user selection for inclusion in the Detection Report.

Paragraph 5. An assay definition file configured to enable a detection instrument to receive a user selection of a first target detection result for inclusion in a Detection Report.

Paragraph 6. An assay definition file configured to enable a detection instrument to receive a user selection of a first target detection result for exclusion from a Detection Report.

Paragraph 7. An assay definition file configured to enable a detection instrument to receive a user selection of a first target detection result for inclusion in a Detection Report and a second target detection result for exclusion from the Detection Report.

Paragraph 8. An assay definition file configured to enable a detection instrument to receive a user selection of a first target detection result for inclusion in a Detection Report and a second target detection result which is not configured for user selection for inclusion in the Detection Report.

Paragraph 9. A non-transitory computer readable storage medium comprising computer executable instructions, the computer executable instructions executable by a processor, the computer executable instructions comprising instructions executable to receive a user selection of a first target for inclusion or exclusion in a Detection Report.

Paragraph 10. A non-transitory computer readable storage medium comprising computer executable instructions, the computer executable instructions executable by a processor, the computer executable instructions comprising instructions executable to receive a user selection of a first target and a second target for inclusion or exclusion in a Detection Report.

### Creating a Dynamic Panel Ordering Rule

After installing a new ADF file, if the ADF file has Dynamic Panel Ordering enabled, the user will need to configure Dynamic Panel Rules.

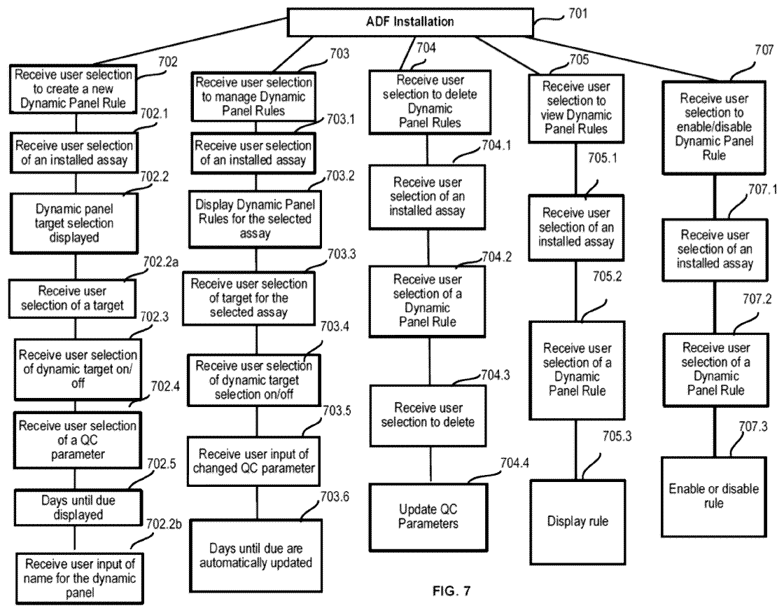

After navigating to the ADF Installation (FIG. 7 at step 701), the user selects between managing Dynamic Panels (FIG. 7 at 703) or creating a new Dynamic Panel (FIG. 7 at step 702) (stated another way, the detection instrument receives a user selection). The ADF Installation is a drop-down menu in the GUI that allows a user to create or manage Dynamic Panels. Selecting an item from a list of options (“Drop down menu”) is a common function of software user interfaces. As a user selects options from a drop-down menu, the subsequent items available for selection in the user interface changes.

In some embodiments, a user can create a new Dynamic Panel by clicking on the create a new Dynamic Panel button (FIG. 7 at step 702). In some embodiments, a user can create a new Dynamic Panel by clicking on the Manage Dynamic Panels button/drop-down (FIG. 7 at step 703). From there, the user clicks on an “edit” button for an assay panel (not shown in FIG. 7) to configure Dynamic Panels specifically for that panel. In some embodiments, only those assays that have a Dynamic Panel defined appear in the manage Dynamic Panels screen. In some embodiments, all the assays that could have a Dynamic Panel defined appear in the manage Dynamic Panels screen whether or not a dynamic rule has been created already.

In some embodiments, selecting “create a new Dynamic Panel” (FIG. 7 at step 702) will produce a dropdown in the GUI that contains all of the installed assays on the system, a.k.a. the “assay dropdown,” also referred to as the “Create a New Dynamic Panel Menu.” In some embodiments, selecting “create a new Dynamic Panel” (FIG. 7 at step 702) will produce an assay dropdown that contains assays for which a Dynamic Panel can be set up on the system (FIG. 7 at step 702.1). In some embodiments, the assay dropdown contains only those assays installed on the system. In some embodiments, the assay dropdown contains only those assays installed on the system that have Dynamic Panel Ordering enabled. For example, if the user only has a respiratory (RP) assay installed on the system, then only the RP assay is displayed in the assay dropdown. In some embodiments, the assay dropdown contains a first assay installed on the system that does not have Dynamic Panel Ordering enabled and a second assay installed on the system that has Dynamic Panel Ordering enabled. In some embodiments, the assay dropdown contains a first assay installed on the system, which has Dynamic Panel Ordering enabled and a second assay installed on the system is not displayed which does not have Dynamic Panel Ordering enabled.

In some embodiments, the assay dropdown (Create a New Dynamic Panel Menu) contains all the assays that could be installed on the system. For example, if the user only has an RP assay installed on the system but could also have a gram-positive, gram-negative, or a fungal assay installed, all will be displayed. Even if a user has not purchased a blood culture panel, the gram-positive, gram-negative, and fungal assays can still appear in the Create a New Dynamic Panel Menu.

In some cases, each assay has a unique colored tag, and the assay dropdown color tag matches the color tag of the assay.

In FIG. 7, at step 702.1, the user selects an installed assay (e.g., RP, gram-positive, gram-negative, or fungal). Stated another way the diagnostic instrument receives the user's selection of an installed assay.

Once the user selects an assay from the Create a New Dynamic Panel Menu (702.1) or the diagnostic system receives the user's selection, the targets on the assay panel are displayed (702.2). In some embodiments, after selecting an assay from the Assay Dropdown, a Dynamic Target Dropdown will be displayed in the GUI which contains all of the targets for the selected assay. In some embodiments, the Dynamic Panel Target Dropdown contains only those targets for the selected assay. For example, if the user has selected the respiratory (RP) assay, then only the RP targets are displayed in the Dynamic Panel Target Dropdown. In some embodiments, all of the targets for all of the assays installed on the system are displayed in the Dynamic Panel Target Dropdown. In some embodiments, the Dynamic Panel Target Dropdown displays as a grid each of the targets on the panel and indicates whether or not the target has been selected for display on the Detection Report, the Dynamic Target selection for each target, the frequency for each target, the rotation for each target and/or the Days Until Due for each target.

In some embodiments, the targets on the assay panel are further displayed in the Dynamic Panel Target Dropdown with the “Dynamic Target” button checked. When the Dynamic Target is checked, it will be displayed in the Detection Report. In some embodiments, each Dynamic Target is checked for display in the report. In some embodiments, each Dynamic Target is unchecked and will not be displayed in the report. In some embodiments, a first plurality of Dynamic Targets is checked and a second, different plurality of Dynamic Targets is unchecked. In some embodiments, the user creates a default (checked or unchecked) to be used for all Dynamic Panel operations. In some embodiments, the user can generate a unique Dynamic Panel for each assay panel. In some embodiments, the default Dynamic Panel has all of the targets checked, i.e., all of the target results will be displayed in the Detection Report. In some embodiments, the default Dynamic Panel has all of the targets unchecked, i.e., all of the target results will not be displayed in the Detection Report. In some embodiments, the default Dynamic Panel has at least one target checked and it cannot be unchecked by the user, i.e., this target will be displayed in the Detection Report and it cannot be selected for non-display by the user; it is a non-configurable target. In some embodiments, the default Dynamic Panel has a first plurality of non-configurable targets checked that cannot be unchecked by the user and a second plurality of Dynamic Targets checked that can be unchecked by the user. In some embodiments, the default Dynamic Panel Rule cannot be modified. In some embodiments, the default Dynamic Panel Rule cannot be modified. i.e., the default Dynamic Panel Rule always has all of the assay targets selected and cannot be changed. In some embodiments, the default Dynamic Panel Rule can be modified.

In some embodiments, the user selects a Dynamic Target (702.2a) such as for example, Adenovirus, Coronavirus (229E, HKU1, NL63, OC43), Human Metapneumovirus, SARS-CoV-2, Human Rhinovirus/Enterovirus, Influenza A, Human Rhinovirus/Enterovirus, Influenza A H1, Influenza A H1-2009, Influenza A H3, Influenza A H1-2009, Influenza B, Parainfluenza 1, Influenza B, Parainfluenza 2, Parainfluenza 3, Parainfluenza 4, Respiratory Syncytial Virus A, Respiratory Syncytial Virus B, Chlamydia pneumoniae, Mycoplasma pneumoniae, or Chlamydia pneumoniae to be included in a Detection Report, stated another way, the detection instrument receives a user selection. In some embodiments, the user does not need to select a Dynamic Target to be included in a Detection Report, they can proceed to the next step without making a selection, i.e., step 702.2a can be skipped.

Once the user selects a Dynamic Target from the Dynamic Panel Target Dropdown (702.2), the target details are displayed. In some embodiments, after selecting a Dynamic Target from the Dynamic Panel Target Dropdown, a Dynamic Panel Target Detail Dropdown will be displayed in the GUI, which contains all of the selected target's Dynamic Panel Target Selection Details and QC details. QC details include but are not limited to expected/predicted result, Accession ID, Dynamic Target selection, Rotation, Lot Alerts (on/off), Requires Valid Control (yes/no), Days Until Due, and combinations thereof for that target. The Dynamic Panel Target Selection Details and QC Details can be created/edited in the Dynamic Panel Target Detail Dropdown or in the Dynamic Panel Target Detail Dropdown. In some embodiments, only the Dynamic Panel Target Selection Details can be created/edited in the Dynamic Panel Target Detail Dropdown or in the Dynamic Panel Target Detail Dropdown. In some embodiments, QC details such as frequency, rotation, Lot alert (on off), Requires Valid Control (yes/no), and Days Until Due are visualized in the Dynamic Panel Target Detail Dropdown but are grayed out or blank, i.e., cannot be changed from this dropdown menu.

In one embodiment, when a new Dynamic Panel Rule is created by a user it is automatically added to the Dynamic Panel database 905.

### Dynamic Target Selection

In the Dynamic Panel Target Detail Dropdown and/or the Dynamic Panel Target Dropdown, the user selects whether the Dynamic Target is selected (on or off) for reporting in a Detection Report; this is called Dynamic Target selection (FIG. 7 at step 702.3). Stated another way, the detection instrument receives the user's instruction for including or excluding a Dynamic Target from a Detection Report.

In some embodiments, the system automatically selects a Dynamic Target for inclusion in a Detection Report, i.e., step 702.3 in FIG. 7 is skipped.

Under Dynamic Target Selection, the user selects whether the Dynamic Target is on or off/selected or not selected/included or excluded from a Detection Report. If the Dynamic Target is on, it is displayed in the Detection Report. If the Dynamic Target is off, it is not displayed in the Detection Report.

In some embodiments, all of the Dynamic Targets on the assay panel have the same Dynamic Target selection. In some embodiments, all of the Dynamic Targets on the assay panel are selected for inclusion in a Detection Report. In some embodiments, all of the targets on the assay panel do not have the same Dynamic Target selection. In some embodiments, all of the targets on the assay panel are not selected for inclusion in a Detection Report. In some embodiments, a first plurality of targets on the assay panel have a first Dynamic Target selection and a second different plurality of targets have a second Dynamic Target selection. In some embodiments, a first plurality of targets on the assay panel are selected for inclusion in a Detection Report and a second different plurality of targets are not selected for inclusion in a Detection Report. In some embodiments, the first Dynamic Target selection and second Dynamic Target selection are different.

In some embodiments, the user selects the “Apply” button when they are done configuring the Dynamic Targets for the assay panel (Step 702.3) and the user is prompted to name the Dynamic Panel (step 702.6). In some embodiments, the user is prompted to name the Dynamic Panel (step 702.6) after step 702.4 and/or 702.5. Initially, the Dynamic Panel Name will be system generated but may be later edited. In step 702.4, the user selects a QC parameter, stated another way, the detection instrument receives a user selection.

In some embodiments, step 702.3 (make a target Dynamic Target selection) occurs before step 702.4 (select a QA parameter). In some embodiments, step 702.3 (make a target Dynamic Target selection) occurs before step 702.4 (select a QA parameter) and the user cannot advance to step 702.4 (select a QA parameter) until after a target Dynamic Target selection (702.3) is selected for each target. In some embodiments, after the user makes a selection (inclusion or exclusion in a Detection Report) for each target, a QA configuration option becomes available, such as run frequency, run rotation, Days Until Due. In some embodiments, step 702.3 (make a target Dynamic Target selection) appears in the Dynamic Panel Target Detail Dropdown before step 702.4 (select a QA parameter) and the user can advance to step 702.4 (select a QA parameter) before a target Dynamic Target selection (702.3) is selected for each target. The QA parameter can include run frequency, rotation order, Lot Alert (on/off), Require Valid Control (yes/no), Days Until Due parameters and combinations thereof.

In some embodiments, step 702.4 (select a QC parameter) occurs before step 702.3 (make a target Dynamic Target selection) and the user cannot advance to step 702.3 (make a target Dynamic Target selection) until after a target rotation (702.4) is selected for each target. In some embodiments, after the user selects a run rotation for each target, a Dynamic Target selection option becomes available (i.e., appears or is no longer grayed out) in the Dynamic Panel Target Detail Dropdown.

In some embodiments, a first target in an assay panel that has Dynamic Panel Ordering enabled is not configurable. In some embodiments, a first target in an assay panel that has Dynamic Panel Ordering enabled is not configurable (i.e., cannot be selected or deselected for reporting the target result on a Detection Report) and a second target is configurable (i.e., can be selected or deselected for reporting the target result on a Detection Report). In some embodiments, a first plurality of targets in an assay panel that has Dynamic Panel Ordering enabled is not configurable and a second plurality of targets is configurable. When a target is not configurable, it is required and included in the Detection Report.

### Target Groupings

In some embodiments, Dynamic Targets are selected independently of one another. In some embodiments, Dynamic Targets are grouped when creating a Dynamic Panel Rule. In these situations, the targets in the Dynamic Panel Target Groupings are either all selected or all deselected for reporting in a Detection Report.

In some embodiments, if a first Dynamic Target is selected, a second Dynamic Target must be deselected. In some embodiments, if a first Dynamic Target is selected, a second Dynamic Target must be selected. In some embodiments, Dynamic Target Groups are defined in the AAM. In some embodiments, Dynamic Target Groups are defined in the ADF.

### Effect on Dynamic Target Rules

In some embodiments, step 702.4 is skipped. Regardless of whether the user makes an edit to a QC parameter or not, the Days Until Due is automatically updated. In some embodiments, the Days Until Due is automatically updated as soon as the user makes a selection for the Dynamic Target (selected or deselected). In some embodiments, the Days Until Due is automatically updated only after the user has made a selection for all of the Dynamic Targets on the assay panel. In some embodiments, the user selects the “Apply” button when they are done configuring the Dynamic Targets/Dynamic Panel Rule for the assay panel (Step 702.3) and the QC parameters are automatically updated based on the Dynamic Target selection (on/off)/Dynamic Panel Rule. In some embodiments, the new Dynamic Panel Rule is automatically saved.

In some embodiments, if a Dynamic Panel Target is on (will be included on the Detection Report), the QC parameters are not affected. In some embodiments, if a Dynamic Panel Target is on (will be included on the Detection Report), the QC parameters are affected. For example, the Days Until Due is automatically updated, i.e., if a Dynamic Panel Target is on (will be included in the Detection Report), the Days Until Due in the Dynamic Target Rules database is updated to reflect its QC due date based on the other Dynamic Targets on the panel. In some embodiments, if a Dynamic Panel Target is on (will be included in the Detection Report), the QC parameters for that target are updated to indicate the QC testing is needed for that target.

In some embodiments, if a Dynamic Panel Target is off (will not be included in the Detection Report), the QC parameters are not affected. In some embodiments, if a Dynamic Panel Target is off (will not be included in the Detection Report), the QC parameters for that target are automatically updated. For example, the Days Until Due is automatically updated, i.e., if a Dynamic Panel Target is off (will not be included in the Detection Report), the Days Until Due in the Dynamic Target Rules database is updated to “null” meaning that the target will not be required for QC. In some embodiments, if a Dynamic Panel Target is off (will not be included in the Detection Report), the QC parameters for that target are updated to indicate the QC testing is not needed for that target.

### Configuring Multiple Dynamic Panels

In some embodiments, each assay panel has a single Dynamic Panel Rule. In some embodiments, each assay panel has more than one Dynamic Panel Rule. In some embodiments, a first assay panel has one Dynamic Panel Rule and a second assay panel has more than one Dynamic Panel Rule. In some embodiments, a first assay panel has more than one Dynamic Panel Rule and a second assay panel has more than one Dynamic Panel Rule.

To create a second Dynamic Panel Rule, the user follows the same steps as described above. Generally, the user navigates to the ADF Installation (FIG. 7 at step 701), the user selects between managing Dynamic Panels (FIG. 7 at 703) or creating a new Dynamic Panel (FIG. 7 at step 702). The user clicks on the Create a new Dynamic Panel button (FIG. 7 at step 702). The user then selects an installed assay. In some embodiments, if the user selects an installed assay, and there is already a Dynamic Panel Rule for that assay, the GUI allows the user to select between creating a new Dynamic Panel Rule or editing the Dynamic Panel Rule that already exists (not shown in FIG. 7). If the user selects the Dynamic Panel Rule that already exists, the user proceeds with step 703.3. If the user selects creating a new Dynamic Panel Rule, the user continues on with step 702.2.

If a first Dynamic Panel Rule already exits, when the user selects a name for a new second Dynamic Panel Rule, the system will compare the names of the first Dynamic Panel Rule and second Dynamic Panel Rule to ensure that the second Dynamic Panel Rule name and first Dynamic Panel Rule name do not overlap. If they are the same, the user will be prompted to select a new name for the second Dynamic Panel Rule or reassign the new rule to the old name (i.e. replace the old rule). In some embodiments, the system assigns a name to the new Dynamic Panel Rule automatically. In some embodiments, the system assigns a name to the new Dynamic Panel Rule automatically and a user can then modify the name.

In some embodiments, new Dynamic Panel Rules cannot conflict with previously created Dynamic Panel Rules. In some embodiments, new Dynamic Panel Rules can conflict with previously created Dynamic Panel Rules. In some embodiments, new Dynamic Panel Rules are completely independent of previously created Dynamic Panel Rules.

In some embodiments, the GUI displays all of the associated Dynamic Panel Rules for an assay panel on a single interface. In this way, different Dynamic Panel Rules can be compared to see what targets are selected for reporting, which are deselected for reporting, and/or which targets are non-configurable.

Creating Dynamic Target Rules can be further understood by the following numbered paragraphs:

Paragraph 1. A method for creating a Dynamic Panel Rule on a detection instrument, the method comprising:
receiving by the detection instrument a user selection of a first installed assay; receiving by the detection instrument the user selection of a first target from the first installed assay; receiving by the detection instrument the user selection for the display of a first target result on a Detection Report for the first target thereby generating a first Dynamic Target; and generating by the detection instrument a Dynamic Panel Rule based on the selection of the first Dynamic Target.

Paragraph 2. The method for creating a Dynamic Panel Rule on a detection instrument of paragraph 1, further comprising: receiving by the detection instrument the user selection of a second target from the first installed assay; receiving by the detection instrument the user selection for the display of a second target result on a Detection Report for the second target thereby generating a second Dynamic Target; and generating by the detection instrument the Dynamic Panel Rule based on the first Dynamic Target and the second Dynamic Target.

Paragraph 3. The method for creating a Dynamic Panel Rule on a detection instrument of any preceding paragraph, further comprising updating by the detection instrument the QC parameters to reflect the frequency, rotation order, Lot Alert (on/off), Require Valid Control (yes/no) and/or Days Until Due based on the Dynamic Panel Rule.

Paragraph 4. The method for creating a Dynamic Panel Rule on a detection instrument of preceding paragraphs 2 and 3, wherein the first Dynamic Target is displayed on the Detection Report but the second Dynamic Target is not displayed on the Detection Report.

Paragraph 5. A method for creating a Dynamic Panel Rule on a detection instrument, the method comprising:
providing by the detection instrument a ADF installation drop-down wherein there is at least one installed assay and no Dynamic Panel Rules; displaying by the detection instrument “there are no Dynamic Panel Rules”; and displaying by the detection instrument a “create a new Dynamic Panel Rule button.”

Paragraph 6. A method for creating a Dynamic Panel Rule on a detection instrument, the method comprising:
providing by the detection instrument a ADF installation drop-down wherein there is at least one installed assay and at least one installed Dynamic Panel Rule for the assay; displaying by the detection instrument an assay drop down for the at least one installed assay having at least one installed Dynamic Panel Rule and at least one installed assay not having at least one installed Dynamic Panel Rule; and displaying by the detection instrument a “create a new Dynamic Panel button” and a “manage a Dynamic Panel button.”

Paragraph 7. A method for creating a Dynamic Panel Rule on a detection instrument, the method comprising:
receiving by the detection instrument a user selection of a first installed assay; displaying by the detection instrument an assay drop down comprising the Dynamic Panel Rules for the selected assay.

Paragraph 8. The method for creating a Dynamic Panel Rule on a detection instrument of paragraph 7, wherein displaying by the detection instrument an assay drop down comprising the Dynamic Panel Rules further comprising displaying whether a first Dynamic Target will be reported on a Detection Report.

Paragraph 9. A method for creating a Dynamic Panel Rule on a detection instrument, the method comprising:
automatically populating, by the detection instrument, a Dynamic Panel Rule for an installed assay.

Paragraph 10. A diagnostic instrument based Dynamic Panel reporting system comprising: a diagnostic instrument having a processor, memory, and access to computer readable media; a computer program stored on computer readable media having a Dynamic Panel Rule module; and a user interface displayed on a monitor for interaction with said computer program.

Paragraph 11. The diagnostic instrument based Dynamic Panel reporting system of paragraph 10, further comprising a network connection between the diagnostic instrument and a computer network for allowing remote access to the computer program.

Paragraph 12. A method for creating Dynamic Panel Ordering on a detection instrument, the method comprising: receiving by the detection instrument a user selection of an assay protocol; receiving by the detection instrument a user selection to add a first Dynamic Panel Rule; receiving by the detection instrument a user selection of a plurality of targets to configure the first Dynamic Panel Rule; receiving by the detection instrument a user selection to apply the Dynamic Panel Rule thereby creating Dynamic Panel Ordering on a detection instrument.

Paragraph 13. The method of paragraph 12 further comprising: receiving by the detection instrument a user selection to add a second Dynamic Panel Rule; and receiving by the detection instrument a user selection of a plurality of targets to configure a second Dynamic Panel Rule.

Paragraph 14. A diagnostic instrument based Dynamic Panel reporting system comprising: a diagnostic instrument having a processor, memory, and access to computer readable media; a computer program stored on computer readable media having a Dynamic Panel Rule.

Creating Dynamic Target Rules can be further understood by the following numbered paragraphs:

Paragraph 1. A detection instrument with at least one Dynamic Panel Rule stored on the detection instrument.

Paragraph 2. A pathogen detection device comprising: a pathogen detector circuit configured to detect a target analyte in a patient sample, determine an identity of a pathogen from the target analyte, and generate a Detection Result; and a decision reporting circuit configured to report the Detection Result for the pathogen by application of a plurality of configurable rules to the Detection Result, wherein each of the configurable rules comprises a corresponding logic expression, the logic expression indicating if the Detection Result is to be included in a Detection Report.

Paragraph 3. The pathogen detection device of Paragraph 2, further comprising a communications interface, wherein the decision reporting circuit is configured to cause the corresponding decision reporting information of the rule to be transmitted to a LIS host via the communications interface.

Paragraph 4. The pathogen detection device of paragraph 1, wherein the decision reporting circuit is configured to allow a user to modify any of the rules via a graphical user interface.

Paragraph 5, The pathogen detection device of paragraph 1, wherein the pathogen is a gastrointestinal pathogen.

Paragraph 6. The pathogen detection device of paragraph 1, wherein the configurable rules are established by a user selecting from a selectable data elements to define “on” or “off” selections.

Paragraph 7. The pathogen detection device of paragraph 1, wherein configurable rules can be modified and the date of the modification and name of the modifier are stored in a logging module.

Paragraph 8. The pathogen detection device of paragraph 1, wherein the rules are applied based on a user selectable scope of application.

Paragraph 9. A non-transitory computer readable storage medium comprising computer executable instructions, the computer executable instructions executable by a processor, the computer executable instructions comprising: instructions executable to receive an identity of a pathogen detected in a patient sample by a detection device; and instructions executable to generate a detection reporting information from the identity of the pathogen detected by the detection device, the Detection Reporting information generated by an application of a set of rules to the identity of the pathogen, wherein each rule in the set of rules includes a corresponding condition for including a corresponding pathogen result in a Detection Report.

Paragraph 10. A pathogen detection device comprising: a display device; and a processor configured to: display, on the display device, a graphical user interface that provides a field for user input; defining a rule based on the user input; apply the rule to a Detection Result; evaluating whether the rule is satisfied; and in response to the rule being satisfied, include the Detection Result in a Detection Report.

Paragraph 11. A pathogen detection device comprising: a display device; and a processor configured to: display, on the display device, a graphical user interface that provides a field for user input; defining a rule based on the user input; apply the rule to a Detection Result; evaluating whether the rule is satisfied; and in response to the rule not being satisfied, not including the Detection Result in a Detection Report.

Paragraph 12. A pathogen detection device comprising: a display device; and a processor configured to: display, on the display device, a graphical user interface that provides a field for user input; defining a rule based on the user input; apply the rule to a target Detection Result; evaluating whether the rule is satisfied; and in response to the rule not being satisfied, not including the target in a Detection Report.

Paragraph 16. The pathogen detection device of paragraphs 10, 11, or 12, wherein the graphical user interface provides the field using a drop-down menu.

### Manage Dynamic Panel Rules

After a Dynamic Panel Rule has been created (Step 702 in FIG. 7), it can be managed (Step 703 in FIG. 7). As with creating a rule, when editing a rule, the user selects the desired display of the target on the Detection Report.

After navigating to the ADF Installation (FIG. 7 at Step 701), the user selects between creating a new Dynamic Panel Rule (FIG. 7 at Step 702) or managing Dynamic Panel Rules (FIG. 7 at Step 103) from the ADF Installation dropdown (stated another way, the detection instrument receives a user selection).

In some embodiments, selecting “Manage Dynamic Panel Rules” (FIG. 7 at 703) will produce a Manage Dynamic Panel Rules Dropdown which displays assays with Dynamic Panel Rules setup on the system. This is the “Manage Dynamic Panel Rules Menu.” The Manage Dynamic Panel Rules Menu reflects the most recent Dynamic Panel Rule parameters (is a target selected for display in the Detection Report and can also include QC parameters such as expected results, run frequency, target rotation). In some embodiments, each installed assay has a corresponding colored assay tag, and the same color is used in the GUI when the assay name is displayed.

In some embodiments, selecting “Manage Dynamic Panel Rules” (FIG. 7 at Step 703) will produce an Installed Assay Manage Dynamic Panel Rules Dropdown which contains the installed assays on the system (e.g., RP2, gram-positive, gram-negative, or fungal). The user selects the installed assay (FIG. 7 at 703.1) and then a second drop-down menu appears which contains the Dynamic Panel Rules that have been set up on the system for that assay (the Manage Dynamic Panel Rules Menu) and the user selects the Dynamic Panel Rule (FIG. 7 at 703.2), stated another way, the detection instrument receives a user selection. In some embodiments, if there is only one Dynamic Panel Rule set up on the system, Step 703.1 is skipped. Step 703.1 can also be skipped if, as in some embodiments, selecting “Manage Dynamic Panel Rules” (FIG. 7 at Step 703) produces an assay dropdown which contains all of the Dynamic Panel Rules that have been set up on the system, i.e., Step 703.1 displays all of the Dynamic Panel Rules set up on the system.

Once the user selects a target (FIG. 7 at Step 703.3) the user can change the Dynamic Target selection (on/off) (FIG. 7 at Step 703.4), QC parameter (FIG. 7 at Step 703.5) or both. When the Dynamic Target selection (on/off) (FIG. 7 at Step 703.4) and/or QC parameter (FIG. 7 at Step 703.5) is changed, the Days Until Due are automatically updated (FIG. 7 at Step 703.6). In step 703.3, the user selects a target, stated another way, the detection instrument receives a user selection. In step 703.4, the user changes a dynamic target selection, stated another way, the detection instrument receives a user selection. In step 703.5, the user changes a QC parameter, stated another way, the detection instrument receives user input.

In some embodiments, (when creating a Dynamic Panel Rule or managing a Dynamic Panel Rule) if all of the assay targets are deselected, an alarm will sound or an alert will be given. In this way, a Dynamic Panel Rule cannot have no reportable results.

In some embodiments, the user selects the “Apply” button when they are done editing a Dynamic Panel Rule. In some embodiments, the edited Dynamic Panel Rule is automatically saved. In some embodiments, when a Dynamic Panel Rule is updated, the user selects the “Reset Days Until Due” button, which then updates the Days Until Due.

In some embodiments, when the manage Dynamic Panel Rule is selected from the ADF Installation dropdown, the user can perform one or more of the following: view all Dynamic Panel Rules, print Dynamic Panel Rules, view run history (a report of the targets detected and reported for that assay or panel), edit Dynamic Panel Rules, duplicate Dynamic Panel Rule, and/or delete a Dynamic Panel Rule.

### Managing Dynamic Targets

In some embodiments, every Dynamic Target can be changed. In some embodiments, not every Dynamic Target can be changed, i.e. some targets are non-configurable.

In some embodiments, every Dynamic Target can be changed but one Dynamic Target must always be selected in an assay panel. Otherwise, after a cartridge is processed, no results would be reported on the Detection Report.

In some embodiments, if an external control has not been run for a target, a user may not deselect the target from the Dynamic Panel Rules. In this way, a user cannot bypass quality control by defining custom Dynamic Panel Rules. In some embodiments, if an external control has not been run, a user may deselect it from the Dynamic Panel Rules. In this way, a control alert can be bypassed by defining custom Dynamic Panel Rules.

### Editing a Dynamic Panel Rule as a Supervisor

In some embodiments, different assay panels can only be changed by someone with the proper credentials, such as a supervisor or administrator. In some embodiments, different targets on an assay panel can only be changed by someone with the proper credentials, such as a supervisor or administrator. In FIG. 7, there is a step before Step 703.1 (select an installed assay) or before Step 703.3 (select a target), or before Step 703.4 (change Dynamic Target selection), or before Step 703.5 (change QC parameter). In these embodiments, the system will ask the supervisor to enter a passcode or scan a badge before selecting an installed assay, selecting a target, changing a Dynamic Target selection, or changing a QC parameter. After the supervisor has entered the passcode or swiped a badge, they can select an installed assay and proceed through the steps to manage a Dynamic Panel Rule. In some embodiments, if the supervisor does not enter a passcode or swipe a badge, the Dynamic Panel Rule cannot be changed.

In some embodiments, the supervisor can edit all of the targets on a Panel. In some embodiments, the supervisor cannot edit all of the targets on a Panel. In some embodiments, the supervisor can edit a subset of targets on a Panel. Edit means selecting or deselecting a target for inclusion or exclusion on a Detection Report.

In some embodiments, if a Dynamic Panel Rule is modified the Dynamic Panel name must also be modified. In some embodiments, if a Dynamic Panel Rule is modified the Dynamic Panel name must also be modified and the modifications will not be saved until a new name is applied to the Rule. This allows the user to track exactly what targets were reported when. If the original Dynamic Panel Rule is no longer needed, it can be disabled. But, the Dynamic Panel Rule can always be viewed in the Dynamic Panel Rule Database 905 for reference.

Managing Dynamic Panel Rules can be further understood by the following numbered paragraphs:

Paragraph 1. A method for managing Dynamic Panel Rules on a detection instrument, the method comprising:
receiving by the detection instrument a user selection of a first Dynamic Panel Rule; receiving by the detection instrument user modification instructions for a first target on the first Dynamic Panel Rule thereby creating a first modified Dynamic Panel Rule; and processing by the instrument a sample cartridge according to the first modified Dynamic Panel Rule.

Paragraph 2. The method for managing Dynamic Panel Rules on a detection instrument of the preceding paragraph, wherein the Days Until Due for the first target is automatically updated by the detection instrument when the modified instructions for a first target for the first Dynamic Panel Rule are received.

### Deleting an Dynamic Panel Rule

In some embodiments, an Dynamic Panel Rule can be deleted. This can be accomplished at the individual rule level, or as part of a bulk action.

### Delete an Individual Dynamic Panel Rule

After navigating to the ADF Installation (FIG. 7 at Step 701), the user selects between creating a new Dynamic Panel Rule (FIG. 7 at Step 702), managing Dynamic Panel Rules (FIG. 7 at 103) or deleting Dynamic Panel Rules (FIG. 7 at 104) from the ADF Installation dropdown (stated another way, the detection instrument receives a user selection).

From the ADF Installation (FIG. 7 at Step 701) drop-down, the user selects Delete Dynamic Panel Rule (FIG. 7 at Step 704) to delete a Dynamic Panel Rule. In some embodiments, selecting “Delete Dynamic Panel Rule” (FIG. 7 at Step 704) will produce an assay dropdown which contains a list of all the installed assays that have a Dynamic Panel Rule. (FIG. 7 at Step 704.1). Selecting an assay will produce an dropdown which contains a list of all the Dynamic Panel Rules associated with that assay. (FIG. 7 at Step 704.2). The user selects the Dynamic Panel Rule (FIG. 7 at Step 704.2) and deletes it (FIG. 7 at Step 704.3). This will delete the Dynamic Panel Rule and all associated Dynamic Target Rules used to generate the Dynamic Panel Rule. In step 704.1, the user selects an assay, stated another way, the detection instrument receives a user selection. In step 704.2, the user selects a Dynamic Panel Rule, stated another way, the detection instrument receives a user selection. In step 704.3, the user selects “delete”, stated another way, the detection instrument receives a user instruction.

### To Delete Multiple Dynamic Panel Rules:

From the ADF Installation (FIG. 7 at Step 701) drop-down, the user can select an assay and hit a delete button (stated another way, the detection instrument receives a user instruction). This will delete all of the Dynamic Panel Rules associated with the assay.

From the ADF Installation (FIG. 7 at Step 701) drop-down, the user can select multiple Dynamic Panel Rules to delete and selects “delete” to delete all of the selected Dynamic Panel Rules.

In some embodiments, all of the Dynamic Panel Rules can be deleted for an assay. In some embodiments, when all of the Dynamic Panel Rules are deleted, the system will process a sample cartridge by reporting a result for each target on the assay panel. In some embodiments, when all of the Dynamic Panel Rules are deleted, the system will process a sample cartridge by reporting no results for each target on the panel.

If a Dynamic Panel Rule is deleted the QC parameters are automatically updated.

Deleting Dynamic Panel Rules can be further understood by the following numbered paragraphs:

Paragraph 1. A method for deleting a Dynamic Panel Rule on a detection instrument, the method comprising:
selecting from a menu a first installed assay; selecting a first Dynamic Panel Rule from the menu; and
deleting the first Dynamic Panel Rule from the menu.

Paragraph 2. The method for deleting a Dynamic Panel Rule on a detection instrument of any preceding paragraph, wherein the Days Until Due for a target in the first installed assay is automatically updated.

Paragraph 5. A method for deleting a Dynamic Panel Rule on a detection instrument, the method comprising:
receiving by the detection instrument a user selection of a first installed assay from a menu; displaying by the detection instrument a plurality of Dynamic Panel Rules associated with the first installed assay; receiving by the detection instrument the user selection of a first Dynamic Panel Rule; receiving by the detection instrument the user instructions deleting the Dynamic Panel Rule; and deleting by the detection instrument the first Dynamic Panel Rule.

### Viewing a Dynamic Panel Rule

In some embodiments, Dynamic Panel Rules can be viewed. After navigating to the ADF Installation (FIG. 7 at Step 701), the user selects between managing Dynamic Panel Rules (FIG. 7 at 103) creating a new QC (FIG. 7 at Step 702), deleting a Dynamic Panel Rule (FIG. 7 at 104) or viewing Dynamic Panel Rules (FIG. 7 at 705) (stated another way, the detection instrument receives a user selection).

### Viewing an Individual Dynamic Panel Rule

From the Manage ADF Installation (FIG. 7 at Step 701) drop-down, the user selects View Dynamic Panel Rules (FIG. 7 at Step 705). In some embodiments, selecting “View Dynamic Panel Rules” (FIG. 7 at Step 705) will produce an assay dropdown which contains a list of all the installed assays that have a Dynamic Panel Rule. (FIG. 7 at Step 705.1). The user selects the assay from the drop-down which then displays a drop-down of the Dynamic Panel Rules for that assay. The user can select a Dynamic Panel Rule (FIG. 7 at Step 705.2) from the drop-down and views it (FIG. 7 at Step 705.3). In step 705.1, the user selects an assay, stated another way, the detection instrument receives a user selection. In step 705.2, the user selects a Dynamic Panel Rule, stated another way, the detection instrument receives a user selection. In step 705.3, the user views a Dynamic Panel Rule, stated another way, the detection instrument displays a Dynamic Panel Rule.

### Viewing Multiple Dynamic Panel Rules

In some embodiments, the user selects the assay (FIG. 7 at Step 705.1) and views all the Dynamic Panel Rules associated with it (FIG. 7 not shown) (stated another way, the detection instrument displays all the Dynamic Panel Rules).

In some embodiments, the QC parameters can also be viewed. In some embodiments, there is a display showing when the Dynamic Panel Rule was last run. In some embodiments, there is a display showing when the Dynamic Panel Rule was last successfully run. In some embodiments, there is a display showing Dynamic Panel Rule information. Dynamic Panel Rule information can include creator username, Dynamic Panel Rule creation date, timestamp the last time the Dynamic Panel Rule was run, bay serial number for the last time the Dynamic Panel Rule was run, bay location for the last time the Dynamic Panel Rule was run, and/or Dynamic Panel Rule violations.

Viewing Dynamic Panel Rules can be further understood by the following numbered paragraphs:

Paragraph 1. A method for viewing a Dynamic Panel Rule on a detection instrument, the method comprising:
selecting from a menu a first installed assay; viewing Dynamic Panel Rules associated with the first installed assay; selecting a first Dynamic Panel Rule for the first installed assay; and viewing the first Dynamic Panel Rule.

Paragraph 2. A method for viewing a Dynamic Panel Rule on a detection instrument, the method comprising:
receiving by the detection instrument a user selection of a first installed assay from a menu; receiving by the detection instrument the user selection of a first Dynamic Panel Rule from the first installed assay; and displaying by the detection instrument the first Dynamic Panel Rule.

### Enable/Disable a Dynamic Panel Rule

In some embodiments, Dynamic Panel Rules can be enabled/disabled. After navigating to the ADF Installation (FIG. 7 at Step 701), the user selects between managing Dynamic Panel Rules (FIG. 7 at 103) creating a new QC (FIG. 7 at Step 702), deleting a Dynamic Panel Rule (FIG. 7 at 104), viewing Dynamic Panel Rules (FIG. 7 at 705) or enabling/disabling a Dynamic Panel Rule (FIG. 7 at 707) (stated another way, the detection instrument receives a user selection).

From the Manage ADF Installation (FIG. 7 at Step 701) drop-down, the user selects Enable/Disable Dynamic Panel Rules (FIG. 7 at Step 707) (stated another way, the detection instrument receives a user selection). In some embodiments, selecting “Enable/Disable Dynamic Panel Rules” (FIG. 7 at Step 707) will produce an assay dropdown which contains a list of all the installed assays that have a Dynamic Panel Rule. (FIG. 7 at Step 707.1). The user selects the assay from the drop-down which then displays a drop-down of the Dynamic Panel Rules for that assay. The user can select a Dynamic Panel Rule (FIG. 7 at Step 707.2) and enables or disables it (FIG. 7 at Step 707.3). This will enable or disable the Dynamic Panel Rule and all associated Dynamic Target rules associated with it. In some embodiments, the user can select more than one Dynamic Panel Rule to enable or disable. In one embodiment, a first Dynamic Rule is enabled and a second Dynamic Rule is disabled. In one embodiment, a first Dynamic Rule is enabled and a second Dynamic Rule is enabled. In step 707.1, the user selects an assay, stated another way, the detection instrument receives a user selection. In step 707.2, the user selects a Dynamic Panel Rule, stated another way, the detection instrument receives a user selection. In step 707.3, the user enables or disables a Dynamic Panel Rule, stated another way, the detection instrument receives a user selection.

In some embodiments, a Dynamic Panel Rule that has been disabled is still viewable in the GUI. In some embodiments, a disabled Dynamic Panel Rule is removed from the Dynamic Panel Ordering Selection presented to users after cartridge scan.

In some embodiments, when a Dynamic Target is deleted from all Dynamic Panel Rules, it is not automatically deselected/disabled from all External Controls. In some embodiments, when a Dynamic Target is deleted (deselected) from all Dynamic Panel Rules, the Dynamic Target is automatically deselected/disabled from all External Controls. When this happens the Days Until Due are automatically updated for the rest of the targets in the rotation.

### Clinical Sample Runs

From the Run Screen, a user initiates a Regular Run through one of the methods available in the run workflow (with barcodes, selecting individual bay, etc.).

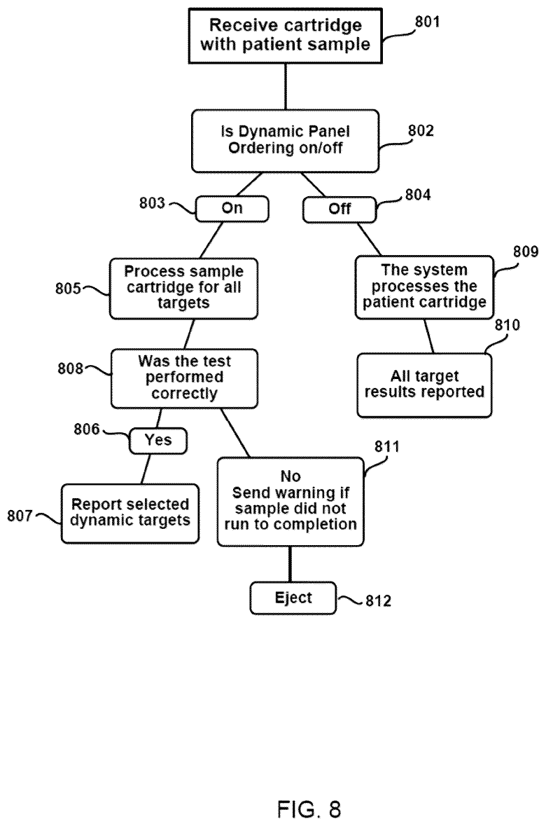

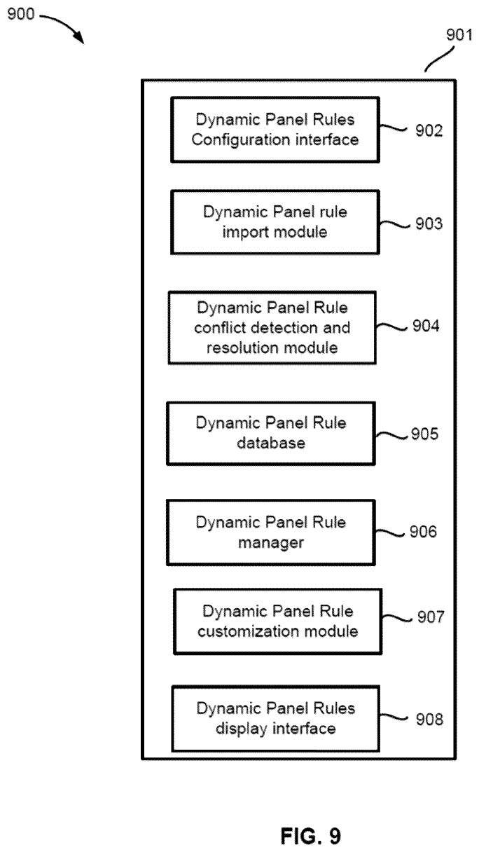

In some embodiments, when a Regular Run is attempted (i.e. a cartridge containing a clinical sample is loaded into the detection instrument, FIG. 8 at step 801), the system will check to see if the “Dynamic Panels” option is turned on. (FIG. 8 at steps 802, 803, and 804). In some embodiments, when a Regular Run is attempted (i.e. a cartridge containing a clinical sample is loaded into the detection instrument, FIG. 8 at step 801), the system scans a bar code which indicates if the “Dynamic Panels” option is turned on for that assay. (FIG. 8 at steps 802, 803, and 804). In some embodiments, when a Regular Run is attempted (i.e. a cartridge containing a clinical sample is loaded into the detection instrument, FIG. 8 at step 801), the system scans a bar code and correlates the bar code with a Dynamic Panel Rule saved in the Dynamic Panel Rule Database (FIG. 9 at 905). The detection results reported on the Detection Report will correlate with the Dynamic Panel Rule saved in the Dynamic Panel Rule Database (FIG. 9 at 905).

In some embodiments, the system will not check if Dynamic Panel Ordering is on until after the sample has been run to completion.

### Dynamic Panel Ordering Selection Mode

In some embodiments, when a Regular Run is attempted (i.e. a cartridge containing a clinical sample is loaded into the detection instrument, FIG. 8 at step 801), the system will check to see if the “Dynamic Panels” option is turned on and if more than one Dynamic Panel Rule is configured for the assay panel.

In some embodiments, instead of correlating the scanned cartridge with a Dynamic Panel Rule, when a cartridge is scanned and Dynamic Panel Ordering option is turned on, the system enters a Dynamic Panel Ordering Selection Mode. In the Dynamic Panel Ordering Selection Mode, the user can select from the available Dynamic Panel Rules associated with that assay. Once the user selects a Dynamic Panel Rule, the system processes the cartridge and reports the results based on the selected Dynamic Panel Rule. In some embodiments, the Dynamic Panel Ordering Selection Mode only displays enabled Dynamic Panel Rules, i.e., disabled or deleted Dynamic Panel Rules are not displayed. In some embodiments, the Dynamic Panel Ordering Selection Mode displays enabled and disabled Dynamic Panel Rules but only enabled rules can be selected. In some embodiments, regardless of Dynamic Panel Rule state (enabled/disabled), there may only be 5 panels shown on the GUI at any given time for user selection in the Dynamic Panel Ordering Selection Mode. In some embodiments, regardless of Dynamic Panel Rule state (enabled/disabled), there may only be 5 or 10, or 40, or 50 panels shown on the GUI at any given time for user selection in the Dynamic Panel Ordering Selection Mode.

If more than one Dynamic Panel Rule exists, the Dynamic Panel Name is used as the selectable panel identifier during the Dynamic Panel Ordering Selection Mode. After selecting a panel, the user confirms their choice by clicking “continue.” Upon continuing, the Run Workflow would resume with inserting the cartridge into the bay.

In some embodiments, if only one Dynamic Panel Rule is configured, the user is not prompted with the Dynamic Panel Ordering Selection Mode and proceeds directly to cartridge insertion following successful scan/input of an accession ID. In such situations, the first Dynamic Panel Rule is automatically applied to the Detection Result.

Regardless of whether the assay target is selected (turned ON) in any of the Dynamic Panels, it is evaluated by the diagnostic instrument, i.e., the system evaluates all of the target and whether they are detected or not-detected in each sample cartridge processed. In contrast, external control targets are not evaluated unless the external control target is selected (turned ON) in at least one Dynamic Panel. For example, if assay target Influenza A is not selected for any Dynamic Panels, it is still evaluated in each and every sample cartridge having Influenza A as part of its panel that is processed by the detection instrument. But, if Influenza A is not on any Dynamic Panels, the system never prompts the user to run an external control for Influenza A. In this way, a target does not undergo external control quality control, but it is still evaluated in the patient sample, but the detection result for the target is never reported.

In some embodiments, if a first target is not selected in any Dynamic Panels for a first assay panel, when a patient sample is processed in the first assay panel comprising the first target, the first target will be evaluated by the detection instrument, but the result will not be reported. In some embodiments, if a first target is not selected in any Dynamic Panels for a first assay panel, an external control is not required for the first target.

Targets that are not selected in any Dynamic Panel are not processed for External Control quality control. Targets that are not selected in any Dynamic Panel are evaluated in every sample cartridge having the deselected target, but the detection result is not reported for the deselected target. Targets that are not selected in any Dynamic Panel obtain a detection result in every sample cartridge having the deselected target, but the detection result is not reported for the deselected target. Stated another way, every target in an assay panel that runs to completion obtains a detection result, but only the detection result for selected targets are reported in a Detection Report. Stated in yet another way, every target in an assay panel that runs to completion obtains a detection result, but the detection results for deselected targets are not reported in a Detection Report. In this way, laboratories can focus on specific targets and not be bogged down with QC testing for targets that are of less clinical relevance and whose results will not be reported.

Processing a Sample Cartridge According to Dynamic Panel Rules can be further understood by the following numbered paragraphs:

Paragraph 1. A method for processing a Sample Cartridge in a detection instrument, the method comprising:
receiving by the detection instrument scanned Sample Cartridge data; receiving by the detection instrument a user selection of a Dynamic Panel Rule; evaluating by the detection instrument whether any External Controls associated with any targets selected for reporting in the Dynamic Panel rule are outstanding; processing the Sample Cartridge in the detection instrument; receiving by the detection instrument a detection result for each target in the sample cartridge; and reporting by the detection instrument target Detection Results only for targets that have been selected for reporting in the Dynamic Panel Rule.

Paragraph 2. A method for processing a Sample Cartridge in a detection instrument, the method comprising:
receiving by the detection instrument scanned Sample Cartridge data; receiving by the detection instrument a user selection of a first Dynamic Panel Rule; evaluating by the detection instrument whether a first Dynamic Target is selected as part of the first Dynamic Panel Rule; evaluating by the detection instrument whether the first Dynamic Target has an outstanding External Control; processing the Sample Cartridge in the detection instrument if the first Dynamic Target does not have an outstanding External Control; and reporting by the detection instrument a Detection Result for the first Dynamic Target if it is selected as part of the first Dynamic Panel Rule.

Paragraph 3. The method of paragraph 2 further comprising: evaluating by the detection instrument whether a second Dynamic Target is selected as part of the first Dynamic Panel Rule; and withholding by the detection instrument a Detection Result for the second Dynamic Target if the second Dynamic Target is not selected as part of the first Dynamic Panel Rule.

Paragraph 4. A method for processing a Sample Cartridge in a detection instrument, the method comprising:
receiving by the detection instrument scanned Sample Cartridge data; evaluating by the detection instrument whether a first Dynamic Panel Rule is associated with the Sample Cartridge data; evaluating by the detection instrument whether a first Dynamic Target is selected as part of the first Dynamic Panel Rule; evaluating by the detection instrument whether the first Dynamic Target has an outstanding External Control; ejecting the Sample Cartridge from the detection instrument if the first Dynamic Target has an outstanding External Control.

In some embodiments, when a Regular Run is attempted (i.e. a cartridge containing a clinical sample is loaded into the instrument, FIG. 8 at step 801), the system will first check to see if the cartridge accession ID matches an External Control Accession ID and if it does not match, the system will then check to see if the “Dynamic Panels” option is turned on and if more than one Dynamic Panel Rule is configured for the assay panel. In some embodiments, if the cartridge accession ID matches an External Control Accession Id, the system will skip panel selection and proceed to cartridge insert as appropriate for the QC Run Workflow. In some embodiments, if the cartridge accession ID matches an sample Accession Id, the system will proceed to Dynamic Panel selection and once a panel is selected it will proceed to cartridge insertion as appropriate for the Sample Run Workflow.

If the “Dynamic Panels” option is turned off (or is not on) (FIG. 8 at step 804), the processing instrument will process the clinical sample (FIG. 8 at step 809) and all of the targets will be reported (FIG. 8 at step 810). If the “Dynamic Panels” option is turned on (or is not off) (FIG. 8 at step 803), the processing instrument will proceed to step 805 (process the sample cartridge for all targets in the panel). In some embodiments, before proceeding to step 805, the user will select a Dynamic Panel Rule to apply to the Detection Results (the instrument will receive a user selection).

Next the system will evaluate if the cartridge was processed to competition (i.e., did not have a DNF or DNS) (FIG. 8 at step 808), if it did not reach competition (FIG. 8 at step 811), a warning is issued. In some embodiments, the clinical sample cartridge will be ejected from the instrument if the clinical sample cartridge is not run to completion (FIG. 8 at step 812).

If the “Dynamic Panels” option is turned on (or is not off) (FIG. 8 at step 803), and the sample cartridge is processed to completion then only targets that have been selected for reporting in the default Dynamic Panel Rule/selected Dynamic Panel Rule will be reported on the Detection Report, all others will not be reported (FIG. 8 at step 807).

In some embodiments, a target in the clinical sample has been detected but, because that target is not selected for reporting in the Dynamic Panel Rule, the detected target will not be reported on the Detection Report. In some embodiments, a first target in the clinical sample is not selected for reporting in the Dynamic Panel Rule, the first target is detected by the detection instrument but not reported on the Detection Report. In some embodiments, a first target in the clinical sample is not selected for reporting in the Dynamic Panel Rule, a second target in the clinical sample is selected for reporting in the Dynamic Panel Rule, the first target is detected by the detection instrument but not reported on the Detection Report and the second target is not detected by the detection instrument and the “not detected” result is reported on the Detection Report. In some embodiments, a first target in the clinical sample is not selected for reporting in the Dynamic Panel Rule, a second target in the clinical sample is selected for reporting in the Dynamic Panel Rule, the first target is detected by the detection instrument but not reported on the Detection Report and the second target is detected by the detection instrument and the “detected” result is reported on the Detection Report.

In some embodiments, a first target in the clinical sample is not selected for reporting in the Dynamic Panel Rule, a second target in the clinical sample is selected for reporting in the Dynamic Panel Rule, the first target is not reported on the Detection Report and the second target is reported on the Detection Report.

In some embodiments, when a Regular Run is attempted, it is prevented if there is no Dynamic Panel Rule associated with the assay panel. In some embodiments, when a Regular Run is attempted, if there is no Dynamic Panel Rule associated with the assay panel, all of the target results (“detected” or “not detected”) will be reported.

In some embodiments, when a Regular Run is attempted, the diagnostic instrument first checks to see if the corresponding external control has been performed successfully, if it has, then it checks to see if there is a Dynamic Panel Rule associated with the assay panel. In some embodiments, when a Regular Run is attempted, the diagnostic instrument first checks to see if the corresponding external controls have been performed successfully, then the system checks if the Lot Alert is turned on (FIG. 3 at step 315) and if the lot control criteria is met, then it checks to see if there is a Dynamic Panel Rule associated with the assay panel. In some embodiments, a user is prompted to select a Dynamic Panel Rule associated with the assay panel.

In some embodiments, when a Regular Run is attempted, the diagnostic instrument first checks to see if there is a Dynamic Panel Rule associated with the assay panel then it checks to see if the corresponding external control has been performed successfully.

In some embodiments, the system does not check to see if there is a Dynamic Panel Rule associated with the assay panel until after the clinical sample has successfully run to completion, i.e., a detection result (detected or not detected) is obtained for each target on the assay panel. This saves processing time so that Dynamic Panel Rules are not evaluated for a sample cartridge that does not successfully run to completion.

Processing a Sample Cartridge According to Dynamic Panel Rules can be further understood by the following numbered paragraphs:

Paragraph 1. A method for processing a Sample Cartridge in a detection instrument, the method comprising:
receiving by the detection instrument scanned Sample Cartridge data; receiving by the detection instrument a user selection of a first Dynamic Panel Rule; processing the Sample Cartridge in the detection instrument; and receiving by the detection instrument a detection result for each target in the Sample Cartridge; reporting by the detection instrument target Detection Results only for targets that have been selected for reporting in the Dynamic Panel Rule.

Paragraph 2. A method for processing a Sample Cartridge in a detection instrument, the method comprising:
receiving by the detection instrument scanned Sample Cartridge data; processing the Sample Cartridge in the detection instrument; receiving by the detection instrument a user selection of a first Dynamic Panel Rule; and reporting by the detection instrument only target Detection Results for targets that have been selected for reporting in the Dynamic Panel Rule.

Paragraph 3. A method for processing a Sample Cartridge in a detection instrument, the method comprising:
receiving by the detection instrument scanned Sample Cartridge data; receiving by the detection instrument a user selection of a first Dynamic Panel Rule; processing the Sample Cartridge in the detection instrument;
reporting by the detection instrument a first target Detection Result for a first target that has been selected for reporting in the Dynamic Panel Rule; and withholding by the detection instrument a second target Detection Result for a second target that has not been selected for reporting in the Dynamic Panel Rule.

Paragraph 5. The method for processing a Sample Cartridge in a detection instrument of paragraphs 1-3, wherein the Days Until Due for a first QC test are automatically updated by the detection instrument when the Sample Cartridge is processed.

Paragraph 6. The method for processing a Sample Cartridge according to paragraph 3, wherein the Dynamic Panel Rule comprises a first target that has been selected for reporting and a second target that has not been selected for reporting.

Processing a Sample Cartridge According to Dynamic Panel Rules can be further understood by the following numbered paragraphs:

Paragraph 1. A sample cartridge comprising: instructions for the reporting by a detection instrument target Detection Results only for targets that have been selected for reporting by a Dynamic Panel Rule.

Paragraph 2. A method to diagnose an infection in a patient, the method comprising: detecting a first microorganism in the patient's sample; detecting a second microorganism in the patient's sample; generating, by the diagnostic instrument, a first Detection Result based on the first detected microorganism and a second Detection Result based on the second detected microorganism; comparing the first Detection Result and second Detection Result to a set of rules; generating a detection report wherein the first Detection Result is included in a Detection Report and the second Detection Result is not included in the Detection Report based on the set of rules.

Paragraph 3. A method for providing Detection Results for a patient that is infected or possibly infected with a microorganism, the method comprising: detecting the microorganism in the patient's sample; generating, by the diagnostic instrument, a Detection Result, which includes an identity of the microorganism; and generating a Detection Report by the diagnostic instrument, wherein generating the Detection Report includes applying, by the diagnostic instrument, a set of configurable rules to the Detection Result, which when evaluated, indicates whether to include the Detection Result in the Detection Report.

Paragraph 4. The method of paragraph 3, wherein the microorganism in the patient's blood causes or is suspected of causing sepsis.

Paragraph 5. The method of paragraph 3, further comprising generating a graphical user interface configured to edit any rule in the set of configurable rules.

Processing a Sample Cartridge According to Dynamic Panel Rules can be further understood by the following numbered paragraphs:

Paragraph 1. A method for managing Dynamic Panel Rules on a detection instrument, the method comprising:
displaying by a detection instrument a first Dynamic Panel Rule;
receiving by the detection instrument a user modification to the first Dynamic Panel Rule;
processing by the instrument a sample cartridge without reference to the Dynamic Panel Rules; and
reporting by the detection instrument results from the processed sample cartridge according to the Dynamic Panel Rules.

Paragraph 2. The method for managing Dynamic Panel Rules on a detection instrument of paragraph 1, wherein the user modification comprises turning off reporting for a first Dynamic Target and turning on reporting for a second Dynamic Target and wherein reporting by the instrument comprises not reporting the first Dynamic Target and reporting the second Dynamic Target.

Paragraph 3. The method for managing Dynamic Panel Rules on a detection instrument of paragraph 1, further comprising updating the Days Until Due for the first Dynamic Panel Rule based on the user modification.

Paragraph 4. The method for managing Dynamic Panel Rules on a detection instrument of paragraph 1, further comprising updating the Days Until Due for a second Dynamic Target based on the user modification to a first Dynamic Target.

### External Controls

In some embodiments, the ISW recognizes that the scanned cartridge is a Control Run not a sample cartridge.

When an external control is processed, all Dynamic Panels are updated with the external control information. Stated another way, when an external control is due and then run, all of the targets in Dynamic Panels are updated with new Days Until Due.

In some embodiments, all targets are reported in External Control Reports whether they are selected or de-selected from reporting in Detection Reports. In some embodiments, only selected Dynamic Targets are reported in External Control Reports. For example, In some embodiments, if a Dynamic Target is deselected in all Dynamic Panel Rules, the Dynamic Target is not reported in any External Control Reports. For example, the target is not included in any external control testing as though it was never in the rotation to begin with. In some embodiments, if a Dynamic Target is not selected in any enabled Dynamic Panel Rules, the Dynamic Target is not reported in any External Control Reports. In some embodiments, if a Dynamic Target is only present in disabled Dynamic Panel Rules, the Dynamic Target is not reported in any External Control Reports.

In some embodiments, if a Dynamic Target is deselected in some Dynamic Panel Rules but is selected in at least one Dynamic Panel Rule, the Dynamic Target is reported in all External Control Reports. In some embodiments, if a Dynamic Target is deselected in some Dynamic Panel Rules but is selected in at least one Dynamic Panel Rule, the Dynamic Target is reported only in External Control Reports linked to the Dynamic Panel Rule. External Control Reports can be linked to a particular Dynamic Panel Rule such that the External Control Report only displays external control run data for the targets in the Dynamic Panel Rule.

In some embodiments, if an External Control is invalid, it is invalid for all Dynamic Panels.

Processing an External Control Cartridge having Dynamic Panel Rules can be further understood by the following numbered paragraphs:

Paragraph 1: A method for processing an External Control cartridge in a detection instrument, the method comprising: recognizing by the detection instrument that a scanned cartridge is an External Control cartridge comprising an external control run protocol; processing the cartridge through the external control run protocol; validating that external run result match expected results; validating the External Control for all associated Dynamic Panels; updating External Control due dates as appropriate for all targets on associated Dynamic Panels.

Paragraph 2: The method of paragraph 1 wherein the detection instrument recognizes that a scanned cartridge is an external control by recognizing a barcode on the cartridge, receiving by the detection instrument user instructions such as entering an external control accession ID or clicking on an external control run button.

Paragraph 3: The method of paragraph 1 wherein External Controls are associated with dynamic protocols when the External Control target is the same as a target on the Dynamic Panel.

### System

FIG. 9 is a schematic diagram of an example of a system 900. The example of the system 900 shown in FIG. 9 includes an integrated diagnostic instrument 901, a Dynamic Panel Rules configuration interface 902, and Dynamic Panel Rule Database 905. Dynamic Panel Rules may be stored in a Dynamic Panel Rule Database 905.

For example, a system implemented by a detection instrument allowing a user to customize reporting is described, the system comprising: a graphical user interface for customizing reporting requirements comprising: a configuration interface 902. The configuration interface 902 may comprise a first GUI interface comprising the schedule of installed assay panels. The configuration interface 902 may comprise a second GUI interface comprising the schedule of targets on a panel. The configuration interface 902 may comprise a third GUI interface comprising an option to select whether a target will be reported in a Detection Report or not. The configuration interface 902 may comprise a fourth GUI interface comprising an option to select QC parameters. The configuration interface 902 may comprise a fifth GUI interface displaying the Days Until Due.

In some embodiments, the GUI interface further comprises a list of specialized Dynamic Panel Rule options that are unique to the detection instrument, the laboratory, the hospital or geographic region of the laboratory/hospital.

In some embodiments, the GUI interface further comprises Dynamic Panel Rule recommendations which are Dynamic Panel Rules recommended for the detection instrument, the laboratory, the hospital or geographic region of the laboratory/hospital. The Dynamic Panel Rule recommendations can be checked on or off from a drop-down menu.

In some embodiments, the instrument 901 comprises a Dynamic Panel Rule import module 903 which manages the importation of Dynamic Panel Rules from various local (laboratory) and/or remote (hospital) databases to the detection instrument. In the illustrated embodiment, the rules import module 903 comprises a conflict detection and resolution module 904.

The Dynamic Panel Rule import module imports Dynamic Panel Rules. The Dynamic Panel Rules may be imported from a local rule databases, remote database and/or existing rules already stored on the detection instrument 901. In some embodiments, the Dynamic Panel Rule Database 905 can hold up to 5 Dynamic Panel Rules for each assay. In some embodiments, the Dynamic Panel Rule Database 905 can hold up to 5, 6, 7, 8, 9, 10, 15, 20, 30, 40, 50 or 100 Dynamic Panel Rules for each assay. In some embodiments, the Dynamic Panel Rule Database 905 must hold at least one Dynamic Panel Rule for each assay installed on the system. In some embodiments, the Dynamic Panel Rule Database 905 is not required to hold at least one Dynamic Panel Rule for each assay installed on the system.

In some embodiments, at any point in time, regardless of whether the panels are active or inactive, no more than 5 Dynamic Panel Rules may exist in the GUI (FIG. 9 at 908). In some embodiments, at any point in time, regardless of whether the Dynamic Panel Rules are active or inactive, no more than 2, or 3, or 4, or 5, or 6 or 7, or 8, or 9, or 10, or 11, or 12, or 15, or 20, or 30, or 40, or 90 panels may exist in the GUI (FIG. 9 at 908). In some embodiments, at any point in time, regardless of whether the panels are active or inactive, more than 5 Dynamic Panel Rules may exist in the Dynamic Panel Rule Database (905) but no more than 5 Dynamic Panel Rules may exist in the Dynamic Panel Rule Display Interface 908. In this way, more than 5 Dynamic Panel Rules are stored on the system but the user only sees up to 5 enabled Dynamic Panel Rules.

In some embodiments, when a new rule is created or imported, the Dynamic Rule conflict detection and resolution module 904 compares the new rule to existing rules already stored on the detection instrument 901 and sends an alert is there is a repeat. Stated another way, the conflict detection and resolution module 904 merges the new Dynamic Panel Rule with existing rules already stored on the detection instrument 901 if they are the same.

In some embodiments, if a newly created Dynamic Panel Rule conflicts with a rule already on the system, the system will generate a new Dynamic Panel Rule for that assay. In this way, each assay can have more than one Dynamic Panel Rule. Once the new Dynamic Panel Rule is created the data is persisted within the Dynamic Panel Rule Database 905 and made accessible to end users via the Dynamic Panel Rule manager 906.

As mentioned above, a conflict detection and resolution module 904 identifies existing rules 905 which match rules being imported from the various sources and sends an alert if there is already a matching Dynamic Panel Rule or creates a new Dynamic Panel Rule if there isn't.

In one embodiment, the various modules shown in FIG. 9 are not separate modules but, rather, separate data structures (e.g., tables) within a relational database.

In some embodiments, the user or the system identifies one or more “Dynamic Panel Classes” within the Dynamic Panel Rule Database 905. As described below, Dynamic Panel Classes are specialized groups of Dynamic Panel Rules which have similar application. The concept of a “Dynamic Panel Class” is a particularly useful abstraction for a detection instrument 901 which runs different types of assays such as Gram positive, Gram negative, fungal or respiratory assays. Some of these assays have unique Dynamic Panel Rules and some of these assays have the same Dynamic Panel Rules. For example, the gram-negative and gram-positive panels could have some targets which are the same. If a Dynamic Panel Class is generated, the target will be treated the same regardless of the assay panel it is on. In one embodiment, a Dynamic Panel Class is formed when a Dynamic Panel Rules matches an existing rule and those rules have the same name and one of the following: detect the same target or run the same control but are for different assay panels. In some embodiments, Dynamic Panel Classes are defined in the AAM. In some embodiments, Dynamic Panel Classes are defined in the ADF.

In some embodiments, to create a Dynamic Panel Class, the user selects and customizes a Dynamic Panel Rule. In one embodiment, this is accomplished with a Dynamic Panel Rule customization module 907. In one embodiment, the Dynamic Panel Rule customization module 907 automatically puts repetitive Dynamic Panel Target rules for different assays into the same Dynamic Panel Class.

### Detection Report

In some embodiments, if Dynamic Panel Ordering is enabled, the Detection Report is modified according to the Dynamic Panel Rule. In some embodiments, if Dynamic Panel Ordering is enabled, the Detection Report header will include the Dynamic Panel Name. In some embodiments, if only the default Dynamic Panel Rule is created (i.e. all targets on the assay panel are reported), the Dynamic Panel Name is not included.

In some embodiments, if Dynamic Panel Ordering is enabled, the Detection Report only identifies targets that have been selected for reporting in the Detection Report and only results are provided for targets that have been selected for reporting in the Detection Report. In some embodiments, if Dynamic Panel Ordering is enabled, the Detection Report identifies all of the targets on the assay panel but only identifies results for the targets that have been selected for reporting in the Detection Report.

In some embodiments, the Detection Report includes the Dynamic Panel name and the last date the Dynamic Panel Rule was modified.

### Target Dependencies

In some embodiments, the detection and reporting of one target depends on the detection of another target. For example,
A first assay panel has a first plurality of targets: T1, T2, T3, T4, and T5.
There's a dependency between T5 and T1 such that if T5 is not detected, it will force T1 to be reported as “not detected” even if T1 is detected by itself.
If the user defines a first Dynamic Panel with a second plurality of targets: T1, T2, and T3.
When the user runs a cartridge and selects the first Dynamic Panel, and the target results are: T1=Detected, T2=Detected, T3=Not Detected, T4=Not Detected, T5=Not Detected.

In some embodiments, the Detection Report shows T1=Detected, T2=Detected, T3=Not Detected, i.e., forcing logic not applied. In this way, because T5 is not included in the Dynamic Panel all rules associated with T5 are excluded from the Dynamic Panel reporting.

In some embodiments, the Detection Report shows T1=Not Detected, T2=Detected, T3=Not Detected, i.e., forcing logic applied. In this way, even though T5 is not included in the Dynamic Panel all rules associated with T5 are still evaluated in the Dynamic Panel reporting.

In some embodiments, Dynamic Panel Rules cannot overrule assay panel logic (forcing logic). In some embodiments, Dynamic Panel Rules can overrule assay panel logic (forcing logic). In some embodiments, a first Dynamic Panel Rule can overrule assay panel logic (forcing logic) but a second Dynamic Panel Rule cannot overrule assay panel logic (forcing logic).

In some embodiments, the Detection Report shows T1=Detected, T2=Detected, T3=Not Detected, i.e., forcing logic not applied and templated comments are not included in the Detection Report. In this way, because T5 is not included in the Dynamic Panel all reporting rules and templated comments associated with T5 are excluded from the Dynamic Panel reporting.

In some embodiments, the Detection Report shows T1=Not Detected, T2=Detected, T3=Not Detected, i.e., forcing logic applied and templated comments are included in the Detection Report. In this way, even though T5 is not included in the Dynamic Panel all reporting rules and templated comments associated with T5 are still evaluated in the Dynamic Panel reporting.

### Test Reporting

In some embodiments, when a sample run finishes, the system will have the following report characteristics:
All targets on the assay panel will be analyzed;
Only the targets selected on the enabled Dynamic Panel at the time the Dynamic Panel is being performed will be visible on the report;
Only the targets selected on the enabled Dynamic Panel at the time the Dynamic Panel is being performed will be transmitted to the LIS (if LIS is enabled).

For example, if a Dynamic Panel exists with 10 targets but only 5 targets are selected, all 10 targets will be evaluated but the detection report will display only the results for the selected 5 targets.

In some embodiments, the results are filtered for epidemiology reporting that allows a user to report the results of all of the targets on the assay panel for epidemiology reporting even if there is a target that has not been selected pursuant to a Dynamic Panel Rule. For example, if a assay panel exists with 10 targets but only 5 targets are selected, all 10 targets will be evaluated but the detection report will display only the results for the selected 5 targets, but all 10 targets will be reported for epidemiology reporting.

### LIS Ordering

In some embodiments, the system automatically generates a pre-defined, unique Dynamic Panel Upload Code for each Dynamic Panel that exists. For example, “Panel 1” shall be referred to as “GI1” in the LIS, “Panel 2” shall be referred to as “GI2” in the LIS etc., regardless of the user-defined name for the Dynamic Panel.

In some embodiments, the LIS is configured to send a PTO to the detection instrument utilizing one of the appropriate Dynamic Panel Upload Codes. In some embodiments, when a PTO is placed for a protocol that contains multiple panels, the Dynamic Panel Upload Code is utilized to distinguish it. In this way, the user does not need to select a Dynamic Panel after a cartridge is scanned, the Dynamic Panel selection information is included in the Dynamic Panel Upload Code.

In some embodiments, a sample is processed according to a first Dynamic Panel Rule and then the Dynamic Panel Rule is modified to create a modified first Dynamic Panel Rule before the sample results are reported. In some embodiments, the Detection Results reported are based on the first Dynamic Panel Rule when the sample was processed. In some embodiments, the Detection Results reported are based on the first modified Dynamic Panel Rule when the result is reported.

In some embodiments, each Dynamic Panel has a system generated LIS Panel Upload code (e.g., GI1).

In some embodiments, if only one Dynamic Panel is configured, only one Dynamic Panel Upload Code will be utilized. In some embodiments, Dynamic Panel Upload Codes cannot be modified by the user. In some embodiments, Dynamic Panel Upload Codes can be modified by the user. In some embodiments, a first Dynamic Panel Upload Codes cannot be modified by the user and a second Dynamic Panel Upload Codes can be modified by the user.

In some embodiments, the LIS Panel Upload Code is displayed to the user during Dynamic Panel setup and can be viewed anytime thereafter. In some embodiments, the LIS Dynamic Panel Upload Code is displayed to the user during panel setup along with the user defined Dynamic Panel name and can be viewed anytime thereafter.

In some embodiments, as additional Dynamic Panels are added, they are assigned a sequential LIS Dynamic Panel Upload Code. For example, by default, the system will always have one panel, and cannot have fewer than one Dynamic Panel. This Dynamic Panel will always be referred to as e.g. “GI1.” If the user adds an additional Dynamic Panel, this Dynamic Panel will have “GI2” Dynamic Panel Upload Code. This would continue in sequence for each Dynamic Panel created. In some embodiments, the LIS Dynamic Panel Upload Code is in sequential order for each assay panel. In some embodiments, the LIS Dynamic Panel Upload Code is in sequential order for each Dynamic Panel regardless of the assay panel it is associated with.

In some embodiments, if a Dynamic Panel is deleted by the user, it is removed from the user interface, but marked as disabled in the backend (Dynamic Panel Rule database 905) so that the LIS Dynamic Panel Upload Code is not recycled and the Dynamic Panel Rule can be referenced later on.

In some embodiments, the Dynamic Panel Upload Code for each Dynamic Panel is visible in the Dynamic Panel ordering configuration screens, such that each Dynamic Panel is clearly identified with its Dynamic Panel Upload Code.

In some embodiments, a global LIS identifier exists, which allows the user to enable bi-directional LIS (e.g. PTO), but still have the user select which Dynamic Panel to run on the instrument.

In some embodiments, when there are multiple Dynamic Panel Rules for a first assay panel, the physician orders the assay panel and selects the Dynamic Panel Rule. In this situation, when the cartridge is scanned by the instrument, the user is not instructed to make a Dynamic Panel selection. Instead, the system automatically runs the cartridge pursuant to the Dynamic Panel Rule selected by the physician.

In some embodiments, when there are multiple Dynamic Panel Rules for a first assay panel, the physician orders the assay panel and selects the Dynamic Panel Rule. In some situations, when the cartridge is scanned by the instrument, the user is instructed to make a Dynamic Panel selection. The user can select a Dynamic Panel that is the same as the physician selected Dynamic Panel Rule or is different from the physician selected Dynamic Panel Rule.

### Templated Comments and Dynamic Panel Rules

“Templated comments” also referred to as “decision reporting information” or “treatment decision reporting information” means decision support that assists clinicians to interpret and/or act on results. Templated comments are described in detail in U.S. Pat. No. 10,935,561, which is hereby incorporated by reference in its entirety.

When a templated comment is created for a target, the templated comment is applied to all Dynamic Panel Rules where that target is selected. In some instances, a templated comment applies to two targets (both targets detected or a first target detected and a second target not detected) in such situations, the templated comment is applied to all Dynamic Panel Rules where either target is selected. In some instances, a templated comment applies to two targets (both targets detected or a first target detected and a second target not detected) in such situations, the templated comment is only applied to Dynamic Panel Rules where both targets are selected.

In some embodiments, a target is deselected, and the target has a templated comment, the templated comment is also automatically disabled.

The system can be further understood by the following numbered paragraphs:

Paragraph 1. A method of analyzing a sample in an instrument having a sample processing bay comprising:
receiving and reading an assay definition file (ADF) from an external source, the ADF enabling reporting rules configuration; receiving reporting rules from a user defining whether a target result will be reported in a detection report; receiving detection results from the sample processing bay; and reporting the detection results based upon the reporting rules.

Paragraph 2. The method according to Paragraph 1 further comprising, reporting by the instrument a first detection result and withholding from the detection report a second detection result.

Paragraph 3. The method according to Paragraph 1, further comprising, reporting a first detection result as “not detected” and withholding a second detection result which is “detected.”

Paragraph 4. The method according to Paragraph 1, wherein a first plurality of detection results received are not reported.

Paragraph 5. The method according to Paragraph 1, wherein a first detection results has reporting logic dependent on a second detection result and wherein the second detection result is not selected for reporting and therefore the first detection result is not reported because the second detection result is not selected for reporting.

The system can be further understood by the following numbered paragraphs:

Paragraph 1. An apparatus for processing and analyzing a sample comprising: a system that operates a sample processing bay and assay analysis module, the system including a processor coupled to a non-transitory computer readable medium, the non-transitory computer readable medium storing instructions directing the processor to: receive and read an assay definition file (ADF) from an external source, the ADF enabling reporting rules configuration; receive reporting rules from the user; send control instructions to the sample processing bay; receive scan data from the sample processing bay; send scan data to the assay analysis module (AAM); receive a detection report from the AAM based on the reporting rules defined by the user; and
report the detection report.

Paragraph 2. The apparatus of Paragraph 2, wherein the reporting rules comprise at least a first target which has been selected for reporting.

Paragraph 3. The apparatus of Paragraph 2, wherein the reporting rules comprise at least a first target which has been selected for reporting and at least a second target that has not been selected for reporting.

Paragraph 4. The apparatus of Paragraph 2, wherein the scan data comprises at least a first scan data for a first target which has been selected for reporting and at least a second scan data for a second target that has not been selected for reporting.

Paragraph 5. The apparatus of Paragraph 2, wherein the scan data comprises at least a first scan data for a first target which has been selected for reporting and at least a second scan data for a second target that has not been selected for reporting and wherein the detection report comprises a detection result for the first target and does not include a detection result for the second target.

Paragraph 6. The apparatus of Paragraph 2, wherein the scan data comprises at least a first detection result for a first target which has been selected for reporting and at least a second detection result for a second target that has not been selected for reporting and wherein the detection report comprises a first detection result for the first target and does not include the second detection result for the second target.

Paragraph 7. A non-transitory computer readable medium for use with an apparatus for processing and analyzing a sample including a processor coupled to a sample processing bay, the non-transitory computer readable medium storing instructions that when executed on by the processor perform steps comprising:

receive and read an assay definition file (ADF) from an external source, the ADF enabling reporting rules configuration; receive reporting rules from a user or LIS; receive detection results from the sample processing bay; and analyze and report results of the detection results based upon the reporting rules.

Paragraph 8. The non-transitory computer readable medium according to Paragraph 7, wherein a first detection result is reported on a detection report and a second result is not reported on the detection report.

Paragraph 9. The non-transitory computer readable medium according to Paragraph 7, wherein a first detection result is “not detected” and it is reported on a detection report and a second result is “detected” and it is not reported on the detection report.

Paragraph 10. An apparatus for processing and analyzing a sample comprising an instrument comprising an Instrument Software Module (ISW) for receiving at least one ADF file enabling reporting rules configuration; a reporting rule module for receiving user instructions defining reporting rules, wherein, when executed by the ISW, the apparatus performs steps comprising detection of a target analyte in the sample and reports detection results for some but not all targets in the sample based on the reporting rules.

Paragraph 11. The apparatus of Paragraph 10, wherein the reporting rules define a first target that is reported on a detection report.

Paragraph 12. The apparatus of Paragraph 10, wherein the reporting rules define a first target that is reported on a detection report and define a second target that is not reported on a detection report.

Paragraph 13. The apparatus of Paragraph 10, wherein the reporting rules define a first target and second target that are reported on a detection report and the first target is detected and the second target is detected.

Paragraph 14. The apparatus of Paragraph 10, wherein the reporting rules define a first target that is reported on a detection report and a second target that is not reported on the detection report wherein the first target is detected and the second target is detected.

Paragraph 15. The apparatus of Paragraph 10, wherein the reporting rules define a first target that is reported on a detection report and a second target that is not reported on the detection report wherein the first target is not detected and the second target is detected.

Paragraph 16. The apparatus of Paragraph 10, further comprising a quality control module the quality control module is automatically updated when the reporting rule module receives user instructions defining reporting rules.

Paragraph 17. The apparatus of Paragraph 10, further comprising a quality control module the quality control module is automatically updated when the reporting rule module receives user instructions defining reporting rules wherein a first target is not reported on the detection report.

Paragraph 18. The apparatus of Paragraph 10, wherein enabling reporting rules configuration comprises some targets that are non-configurable.

Paragraph 19. The apparatus of Paragraph 10, wherein receiving user instructions defining reporting rules comprises receiving a first reporting rule and second reporting rule wherein the first reporting rule conflicts with the second reporting rule.

### Templated Comments—Internal Notes

As described in U.S. Pat. No. 10,935,561, which is hereby incorporated by reference in its entirety, templated comments are sent to the treating physician. Additionally, notes can be applied to the detection result which are also sent to the treating physician. In some situations, it is desirable to include a note internal to the laboratory which is not sent to the treating physician.

An “Internal Note” comment that prints to the detection report but is not sent over the LIS to the treating physician is disclosed. In such situations, the internal note is only visible to the lab tech and their team.

In some embodiments, the detection report comprises an “Internal Note.” The sample-to-answer system may allow the operator to manually include internal notes to the detection report called detection report internal notes. The internal note is not sent over the LIS to the treating physician. The internal note is not sent over the LIS to the hospital. In some embodiments, the internal note is only visible to the lab tech and their team. In some embodiments, the note is included in reports other than the detection report such as epidemiology reports, QC reports, OC reports, cleaning reports etc.

In some embodiments, before the detection report is generated, the system will prompt the user to add an internal note. The internal note can be added to an internal note dialog box. The internal note can be entered manually.

In some embodiments, before the detection report is generated, the system will prompt the user to add an internal note and an external note. The internal note can be added to an internal note dialog box. The external note can be added to an external note dialog box. The internal note can be entered manually. The external note can be entered manually. The internal note is not reported to the physician the external note is reported to the physician.

In some embodiments, before the detection report is printed, the system will prompt the user to select an internal note from a drop down of selectable, pre-populated internal notes.

User notes can be further understood by the following numbered paragraphs:

Paragraph 1. A pathogen detection device comprising: a pathogen detector circuit configured to detect a target analyte in a patient sample, determine an identity of a pathogen from the target analyte, and generate a detection result including the identity of the pathogen; a decision support generator circuit that identifies decision support information and generates a signal based on the decision support information to create a detection report comprising the detection result and the decision support information; a notes circuit configured to display a graphical user interface and to accept user notes and generate a signal based on the user notes to create a detection report comprising the detection result, the decision support information and user notes; and a communications interface, wherein the decision support generator circuit is further configured to cause the decision support information to be transmitted to a Laboratory Information System (LIS) via the communications interface and to cause the user note not to be transmitted to the LIS.

Paragraph 2. The pathogen detection device of Paragraph 1, wherein the decision support generator circuit searches rules stored in a rule repository to detect at least one rule that corresponds to the detection result and in response to detecting the at least one rule, identifies decision support information and generates a signal based on the decision support information to create a detection report comprising the detection result and the decision support information.

Paragraph 3. The pathogen detection device of Paragraph 1, further comprising a Laboratory Information System (LIS) interchange in network communication with a LIS, wherein the LIS interchange is configured to receive the detection report from the decision support generator circuit and convert the detection report into a physician test result report, wherein the physician test result report contains the detection result and the decision support information but does not contain the user notes.

Paragraph 4. A method for providing treatment decision support for a patient having blood that is infected or possibly infected with a microorganism, the method comprising: detecting the microorganism in the patient's blood sample, wherein detecting the microorganism comprises detecting, by a diagnostic instrument, a target analyte in a patient sample; generating, by the diagnostic instrument, a detection result, which includes an identity of the microorganism; receiving, by the diagnostic instrument, user notes; generating decision support information by the diagnostic instrument, wherein generating the decision support information includes applying, by the diagnostic instrument, configurable rules to the detection result; displaying the decision support information on the detection report; and withholding the user notes from the detection report.

Paragraph 5. The method of Paragraph 4, further comprising: converting the detection report into a physician test result report containing both the detection result and the decision support information for transmission to a Laboratory Information System (LIS) over a network and withholding the user notes from the physician test result.

Paragraph 6. A pathogen detection device comprising: a display device; and a processor configured to:
display, on the display device, a graphical user interface that provides a first field for user input and a second field for user input; include decision support information associated with the detection result in a detection report include user input from the first filed in the detection report and withhold the user input from the second field from the detection report.

Paragraph 7. A pathogen detection device comprising: a display device; and a processor configured to:
display, on the display device, a graphical user interface that provides a drop-down menu of a plurality of pre-populated choices for user selection; receive a user selection of a pre-populated choice; include decision support information in a detection report; exclude the user selection of the pre-populated choice from the detection report.

Paragraph 8. A method for processing at least a first patient sample comprising: a) receiving a first patient sample; b) receiving a first test order by a bi-directional LIS interchange in a first file format from a hospital laboratory information system (LIS) for the first patient sample, wherein the bidirectional LIS interchange is on a target analyte detection instrument, and the bi-directional LIS interchange converts the first file format to a second file format; and c) processing the first patient sample in the target analyte detection instrument using the second file format, wherein the target analyte detection instrument generates a first result identifying at least one pathogen in the first patient sample processed by the target analyte detection instrument, wherein the target analyte detection instrument receives a first user note related to the patient sample, first detection result or both, and wherein the target analyte detection instrument receives a second user note related to the patient sample, first detection result or both; and wherein the first detection result is automatically reported from the bi-directional LIS interchange, the first user note is automatically reported from the bi-directional LIS interchange, and second user notes is automatically not reported from the bi-directional LIS interchange.

Paragraph 9. The method of Paragraph 8, wherein the LIS interchange automatically reports the first result to the hospital LIS and automatically withholds the second user notes from the hospital LIS.

Paragraph 10. The method of Paragraph 8, wherein the first test order is in an HL7 or ASTM format and the LIS interchange converts the test order from the HL7 or ASTM format to a CSV format.

Paragraph 11. The method of Paragraph 8, wherein the bi-directional LIS interchange connects to more than one hospital LIS.

Paragraph 12. The method of Paragraph 8, wherein the second user note is reported in a quality control report and/or epidemiology report.

Paragraph 13. The method of Paragraph 8, wherein the second user note is reported in a quality control report and/or epidemiology report and the first user note is not reported in a quality control report and/or epidemiology report.

Paragraph 14. A pathogen detection device comprising: a pathogen detector circuit configured to detect a target analyte in a patient sample, determine an identity of a pathogen from the target analyte, and generate a detection result including the identity of the pathogen; a circuit configured to display a graphical user interface to accept user input related to the patient sample, identity of a pathogen, and/or the detection result; and a reporting circuit configured to generate a detection report comprising patient sample information, the identity of a pathogen, and/or the detection result and not comprising the accepted user input.

Paragraph 15. A pathogen detection device comprising: a pathogen detector circuit configured to detect a target analyte in a patient sample, determine an identity of a pathogen from the target analyte, and generate a detection result including the identity of the pathogen; a first circuit configured to display a graphical user interface to accept first user input related to the patient sample, identity of a pathogen, and/or the detection result; a second circuit configured to display a graphical user interface to accept second user input related to the patient sample, identity of a pathogen, and/or the detection result; and a reporting circuit configured to generate a detection report comprising patient sample information, the identity of a pathogen, the detection result and the first user input but not comprising the accepted second user input.

Paragraph 16. A method for processing at least a first patient sample comprising: a) receiving a first patient sample; b) receiving a first test order by a bi-directional LIS interchange in a first file format from a hospital laboratory information system (LIS) for the first patient sample, wherein the bidirectional LIS interchange is on a target analyte detection instrument, and the bi-directional LIS interchange converts the first file format to a second file format; and c) processing the first patient sample in the target analyte detection instrument using the second file format, wherein the target analyte detection instrument receives a first user comment and generates a first result identifying at least one pathogen in the first patient sample processed by the target analyte detection instrument; and wherein the first result is automatically reported from the bi-directional LIS interchange but the first user comments are not reported from the bi-directional LIS interchange.

Paragraph 17. A method for processing at least a first patient sample comprising: a) receiving a first patient sample; b) receiving a first test order by a bi-directional LIS interchange in a first file format from a hospital laboratory information system (LIS) for the first patient sample, wherein the bidirectional LIS interchange is on a target analyte detection instrument, and the bi-directional LIS interchange converts the first file format to a second file format; and c) processing the first patient sample in the target analyte detection instrument using the second file format, wherein the target analyte detection instrument receives a first user comment, a second user comment and generates a first result identifying at least one pathogen in the first patient sample processed by the target analyte detection instrument; and wherein the first result and second user comments are automatically reported from the bi-directional LIS interchange but the first user comments are not reported from the bi-directional LIS interchange.

### Templated Comments—Rule Order

As described in U.S. Pat. No. 10,935,561, which is hereby incorporated by reference in its entirety, templated comments is a heavily utilized feature for applying user defined comments to reports based on targets detected. However, critical templated comments can get buried when multiple templated comments are applied to a result.

Disclosed herein is a method by which templated comments are prioritized/ordered so that key templated comments appear first.

In some situations, a test result has more than one associated templated comment. In such situations, all templated comments that apply to a particular test result are printed on the detection report. Up until now, only a single templated comment could be printed on a detection report. Or, if multiple templated comments were printed, key results could get buried. The order of the multiple applicable templated comments can now be controlled. For example, In some embodiments, when more than one templated comment is applicable to a detection result, the display of the templated comments on the detection report is configurable. In some embodiments, when more than one templated comment is applicable to a detection result, the display of the templated comments on the detection report is configurable by rule type. In other words, the display of the templated comments is based on the templated comment rule's selectable parameters. For example, there may be laboratory network rules, laboratory rules, hospital network rules, hospital rules or user (aka clinician) rules. When the rules are created the hierarchy of the rule type is assigned. The rule hierarchy is followed when displaying more than one templated comment on a detection result. For example, laboratory network rules are displayed first then laboratory rules then hospital network rules then hospital rules then clinician rules.

Alternatively, templated comments requiring the highest level of authority to create, edit, enable, disable, and/or delete are displayed first on a detection report in the case where more than one templated comment applies to a detection result. For example, rules created by a system administrator are displayed first then rules created by a laboratory overseer then rules created by a hospital network overseer then rules created by a hospital overseer then rules created by a user.

In some embodiments, even if the templated comment conflict, the templated comment from all applicable templated comments are applied to the detection report.

In some embodiments, templated comments from Special Application Rules are displayed on a detection report before templated comments from regular application rules. For example, the scope of a particular rule may be set to be applicable only to a particular account, to a particular hospital, laboratory, etc. The scope of a particular rule may be set to be only applicable for a certain time period (for example special template comments during an outbreak). These types of templated comments from special application rules are displayed first before templated comments from rules which do not have a special application (aka regular rules).

In some embodiments, templated comments from Special Action rules are displayed on a detection report before special application rules or regular application rules. For example, during an outbreak, certain detection results may receive Special Action due to nature of the outbreak and templated comments from the Special Action rules are displayed first.

In some embodiments, templated comments from rules relating to the genus are displayed on a detection report before templated comments from rules relating to the species of the detected microorganism. For example, templated comments applicable to the genus level may be displayed on the detection report before templated comments applicable to the species level, type or gene. In some embodiments, templated comments from rules relating to the genus are displayed on a detection report before templated comments from rules relating to the species of the detected microorganism and templated comments relating to the species are displayed before templated comments associated with resistance gene markers or pan-targets. In some cases, templated comments from the highest category are displayed first, i.e., templated comments from genus rules are displayed before templated comments from species rules which are displayed before templated comments from resistance gene marker rules which are displayed before templated comments from pan-target rules. In some cases if rules conflict the template comments from both or all applicable rules are displayed on the detection report. In some cases if rules conflict the template comments from none of the applicable rules are displayed on the detection report. In some cases template comments from species rules are displayed on the detection report before template comments from genus rules.

In some cases, when a templated comment rule is created it is given a hierarchy such as a red, yellow or green flag rules. In some cases, templated comments from the red category are displayed on the detection report first, then templated comments from yellow rules, then templated comments from green flagged rules.

In some embodiments, when two or more templated comments are to be displayed on a detection report, the user assigns a templated comment display order to each templated comment. In some embodiments, to assign the templated comment display order, the user manually enters a rotation order into the templated comment rotation order selection box, i.e., first, second, third, etc. In some embodiments, by default, the templated comment display order selection boxes are empty, and the user fills them in. In some embodiments, by default, the templated comment display order selection boxes are filled in, and the user only changes the templated comment display order if desired. In some embodiments, to assign templated comments a templated comment display order, the user selects from the available templated comment display orders in a templated comment display order dropdown wherein the number of available templated comment display orders matches the number of templated comments to be displayed.

Templated Comments Rule Order can be further understood by the following numbered paragraphs:

Paragraph 1. A method for providing treatment decision support for a patient having blood that is infected or possibly infected with a microorganism, the method comprising: detecting the microorganism in the patient's blood sample, wherein detecting the microorganism comprises detecting, by a diagnostic instrument, a target analyte in a patient sample; generating, by the diagnostic instrument, a detection result, which includes an identity of the microorganism; and generating decision support information by the diagnostic instrument, wherein generating the decision support information includes applying, by the diagnostic instrument, a set of configurable rules to decision support information, wherein the decision support information is displayed on a detection report according to the set of configurable rules.

Paragraph 2. The method of Paragraph 1., wherein applying the set of configurable rules to the decision support information comprises applying a ranking to each decision support information.

Paragraph 3. A pathogen detection device comprising: a pathogen detector circuit configured to detect a target analyte in a patient sample, determine an identity of a pathogen from the target analyte, and generate a detection result including the identity of the pathogen; a decision support generator circuit configured to generate decision support information for the pathogen by application of a plurality of decision support generator configurable rules to the detection result; and a decision support organizer circuit configured to organize the decision support information by application of a plurality of decision support organizer configurable rules to the decision support information wherein each of the decision support organizer configurable rules comprises a corresponding logic expression, the logic expression indicating in what order the corresponding decision support information is to be included in a detection report.

Paragraph 4. The pathogen detection device of paragraph 3, wherein the decision support organizer circuit is configured to allow a user to modify any of the decision support organizer configurable rules via a graphical user interface.

Paragraph 5. The pathogen detection device of Paragraph 3, further comprising a decision support organizer rule reconciler that is configured to automatically compare a new decision support organizer rule to an existing decision support organizer rule in rule storage.

Paragraph 6. The pathogen detection device of Paragraph 5, wherein if the rule reconciler identifies a conflict between a new decision support organizer rule to an existing decision support organizer rule in rule storage, the new decision support organizer rule is sent to a rule decision support organizer management module.

Paragraph 7. The pathogen detection device of Paragraph 3, wherein the decision support organizer configurable rules are established by a user selecting from a series of selectable data elements to define the order of the decision support organizer rules.

Paragraph 8. The pathogen detection device of Paragraph 3, wherein when a decision support organizer rule is evaluated a first assay target is considered first and then any rule children associated with the first assay target; a second assay target is considered second and then any rule children associated with the second assay target.

Paragraph 9. A non-transitory computer readable storage medium comprising computer executable instructions, the computer executable instructions executable by a processor, the computer executable instructions comprising: instructions executable to receive an identity of a pathogen detected in a patient sample by a detection device; and instructions executable to generate decision support information from the identity of the pathogen detected by the detection device, the decision support information generated by an application of a set of rules to the identity of the pathogen; and instructions executable to generate a decision organization hierarchy from the decision support information generated by an application of a set of decision organization rules to the decision support information, wherein each rule in the set of decision organization rules includes an available templated comment display order in a templated comment display order dropdown wherein the number of available templated comment display orders matches the number of templated comments to be displayed.

Paragraph 10. A pathogen detection device comprising: a pathogen detector circuit configured to detect a target analyte in a patient sample, determine an identity of a pathogen from the target analyte, and generate a detection result including the identity of the pathogen; a decision support generator circuit that receives the detection result and searches rules stored in a rule repository to detect at least one rule that corresponds to the detection result and in response to detecting the at least one rule, identifies decision support information and generates a signal detectable by a decision support organizer circuit; the decision support organizer circuit that receives the decision support information and searches organizer rules stored in an organizer rule repository to detect at least one rule that corresponds to the decision support information and in response to detecting the at least one organizer rule, identifies a hierarchy of decision support information and generates a signal based on the hierarchy of decision support information to create a detection report comprising the detection result and the decision support information displayed according to the hierarchy; and a circuit configured to display a graphical user interface to accept user input and change the hierarchy of the rules stored in the rule repository in response to the user input.

Paragraph 11. A pathogen detection device comprising: a display device; and a processor configured to:
display, on the display device, a graphical user interface that provides a field for user input; defining a rule based on the user input; apply the rule to a first decision support information and a second decision support information; evaluating whether the rule is satisfied; and in response to the rule being satisfied, include first decision support information and a second decision support information in the order defined by the rule in a detection report.

Paragraph 12. The pathogen detection device of paragraph 11, wherein the rule is part of a hierarchy of rules, and wherein defining the rule affects the hierarchy.

Paragraph 13. The pathogen detection device of Paragraph 11, wherein the graphical user interface provides the field using a drop-down menu.

Paragraph 14. A pathogen detection device comprising: a display device; and a processor configured to:
display, on the display device, a graphical user interface that provides a drop-down menu of a plurality of pre-populated choices for user selection; receive a user selection of a pre-populated choice; create a rule, wherein the created rule is one of a plurality of rules in a hierarchy, and wherein creation of the rule alters the hierarchy of the plurality of rules; apply the altered hierarchy of the plurality of rules to a first decision support information and to a second decision support information; and in response to the rule being satisfied, include the first decision support information and second decision support information in a detection report according to the altered hierarchy.

Paragraph 15. The pathogen detection device of Paragraph 14, wherein the processor is further configured to apply the altered hierarchy to the decision support information, wherein a rule higher in the altered hierarchy is applied before a rule lower in the altered hierarchy.

Paragraph 16. The pathogen detection device of claim Paragraph 14, wherein the altered hierarchy comprises rules associated with a genus result and rules associated with a species result; and

wherein the processor is further configured to apply the rules associated with the genus result first and then apply the rules associated with the species result.

Paragraph 17. The pathogen detection device of Paragraph 14, wherein each rule in the altered hierarchy comprises a condition for applying the rule, and wherein the processor is further configured to select which rules to apply based on which conditions are met.

Paragraph 18. The pathogen detection device of Paragraph 14, wherein the conditions comprise one or more of the following: a particular time period, a particular account, a particular hospital, or a particular laboratory.

### Use Cases

User Story: I want to create Dynamic Panels on my available ADF.

### Example 1: Use Case

TABLE 8

| Actor | User |
|---|---|
| Pre- | ADF allows for Dynamic Panel Ordering |
| Conditions | A new Dynamic Panel needs to be created |
| Post- | Multiple panels are created for the applicable ADF, each |
| Conditions | with different target configurations. |
| Flow | On the Assays Available screen, supervisor selects a |
| | protocol |
| | User selects the plus icon to add a new panel |
| | User configures targets per desired need |
| | User chooses to stop, or add additional panels each with |
| | different target selections |
| | User chooses to Apply panel and target selections |
| Exceptions | Only 5 Dynamic Panels can be displayed on the UI at any |
| | given time, regardless of state (enabled/disabled). |

### Example 2: Use Case

User Story: As an Operator I want to run external controls for a protocol that has more than one Dynamic Panel.

TABLE 9

| Actor | Operator |
|---|---|
| Pre- | External Controls are configured for the assay at the |
| Conditions | protocol level (E.g. for the entire Gastrointestinal protocol). |
| | E.g. Pos-A, Pos-B and Neg. |
| | Pos A and B cover all targets. |
| | Multiple Dynamic Panels exist. External controls are due, |
| | or desired to be run. |
| Post- | Valid external controls exist across all configured panels |
| Conditions | for the affected protocol. |
| Flow | A user initiates the External Control run workflow either |
| | by scanning the cartridge from the Run Screen, or by |
| | selecting the Run button from the QC menu. |
| | The system recognizes the run as an External control either |
| | because of the barcode on the cartridge, the user manually |
| | entering the ACCID, or the Run button was used. |
| | The system progresses through the run workflow, asking |
| | the user to insert the cartridge. |
| | The run has started. |
| | Upon completion, the system performs run validation to |
| | ensure result targets match expected targets. |
| | The External Control is Valid. |
| | The system generates an External Control Report |
| | The system updates the External Control due dates as |
| | appropriate. |
| | The system validates the External Control for the entire |
| | relevant protocol. |
| Exceptions | The user does not select a Dynamic Panel if an external |
| | control accession id is identified. Stated another way if the |
| | system recognizes a control run, it does not prompt the user |
| | to select a Dynamic Panel (as it would during a clinical |
| | run). |
| | A Dynamic Target is evaluated if the target is turned ON in |
| | any of the Dynamic Panels. The target does not need to be |
| | ON in ALL Dynamic Panels - just one. |
| | Targets that are not evaluated (not selected in a Dynamic |
| | Panel Rule) are not shown on the External Control Report |
| | External controls need only be run at the protocol level, and |
| | are not evaluated at the Dynamic Panel level. |
| | If an External Control is Invalid, it is Invalid for all |
| | Dynamic Panels. |
| | If the target is disabled (in this case disabled on ALL |
| | panels), it is not reported |

### Example 3: Use Case

TABLE 10

| Actor | Operator |
|---|---|
| Pre- | Detection Instrument is properly integrated to the customer |
| Conditions | LIS system |
| | LIS is enabled |
| | The ADF being scanned has Dynamic Panel Ordering |
| | enabled |
| | The LIS system has sent a test order to ePlex and it was |
| | properly received with a unique LIS Upload Code specific |
| | to one of the programmed Dynamic Panel |
| Post- | A cartridge is scanned and the panel is automatically |
| Conditions | selected allowing the operator to skip panel selection and |
| | move forward in the run workflow with cartridge insertion. |
| Flow | An Operator scans a cartridge |
| | The system recognizes the cartridge and matches it to a |
| | PTO |
| | The system asks the user to insert the cartridge in to a bay |
| | The system progresses through the run workflow including |
| | pre-flight and test start. |
| Exceptions | If the Dynamic Panel has a Dynamic Target selected and |
| | Prompted, the user will still receive a prompt for that target |
| | after cartridge scan and prior to inserting into a bay. |

### Example 4: Use Case

User Story: As an Operator, I want to pull a historical test report on a Dynamic Panel that has the same Dynamic Panel name but different target configuration as compared to the historical report.

TABLE 11

| Actor | Operator |
|---|---|
| Pre- | An ADF with Dynamic Panels is installed |
| Conditions | A Dynamic Panel is configured with customizable targets |
| | (timepoint A) |
| | A run has successfully completed utilizing a specific |
| | Dynamic Panel (e.g. Panel 1) |
| | Time passes |
| | The target configuration is changed but the Dynamic Panel |
| | name is not (E.g. Panel 1) (Timepoint B) |
| Post- | A report is displayed that shows only the targets that were |
| Conditions | configured on the panel at the time the panel was run (e.g. |
| | Timepoint A). |
| Flow | A user navigates to Test Results |
| | Optional: A user is able to filter for a specific Dynamic |
| | Panel |
| | A user selects an old report created during Timepoint A |
| | The system generates a report that displays only the targets |
| | that were configured at the time the cartridge was run. |
| | The Detection Report Header includes a line for Dynamic |
| | Panel Name, which is the name that was assigned to the |
| | panel at the time the panel was run. |
| | Nice to have: A filter exists under Test Results and |
| | Epidemiology reporting that allows user to filter for |
| | Dynamic Panel Name |
| Exclusions | The Dynamic Panel filter on Test Results, if implemented, |
| | will only allow users to filter for Dynamic Panel Names |
| | that are currently active. |

### Example 5: Use Case

User Story: As an Operator, I want to manually release a Detection Report on a Dynamic Panel that has a modified target configuration as compared to the historical report I wish to send at the time it was created.

TABLE 12

| Actor | Operator |
|---|---|
| Pre- | An ADF with Dynamic Panels is installed |
| Conditions | A Dynamic Panel is configured with customizable targets |
| | (timepoint A) |
| | A run has successfully completed utilizing a specific |
| | Dynamic Panel (e.g. Panel 1) |
| | Time passes |
| | The target configuration is changed but the Dynamic Panel |
| | name is not (E.g. Panel 1) (Timepoint B) |
| | I wish to manually release to LIS a historical report created |
| | prior to the panel modification |
| Post- | A report is released sending only the targets that were |
| Conditions | selected at the time the report was generated |
| Flow | A user navigates to Test Results |
| | A user selects an old report created during Timepoint A |
| | A user selects “Send to LIS” |
| | The system sends to LIS only the targets that were selected |
| | at the time the report was created. |

### Example 6: Use Case

User Story: As an Operator, I want a Pending Test Order to automatically select the appropriate Dynamic Panel.

TABLE 13

| Actor | Operator |
|---|---|
| Pre- | External Controls are configured. |
| Conditions | A protocol exists with multiple panels configured. External |
| | controls are due, or desired to be run. |
| Post- | Valid external controls exist across all configured panels |
| Conditions | for the affected protocol. |
| Flow | A user initiates the External Control run workflow either |
| | by scanning the cartridge from the Run Screen, or by |
| | selecting the Run button from the QC menu. |
| | The system recognizes the run as an External control either |
| | because of the barcode on the cartridge, the user manually |
| | entering the ACCID, or the Run button was used. |
| | The system progresses through the run workflow, asking |
| | the user to insert the cartridge. |
| | The run is started. |
| | Upon completion, the system performs run validation to |
| | ensure result targets match expected targets. |
| | The External Control is Valid. |
| | The system generates an External Control Report |
| | The system updates the External Control due dates as |
| | appropriate. |
| | The system validates the External Control for the entire |
| | relevant protocol. |
| Exceptions | The user does not select a Dynamic Panel if an external |
| | control accession id is identified |
| | A Dynamic Target is not evaluated if the target is not |
| | turned ON in any of the Dynamic Panels. |
| | Targets that are not evaluated are not shown on the External |
| | Control Report |
| | External controls need only be run at the protocol level, |
| | and are not evaluated at the Dynamic Panel level. |
| | If an External Control is Invalid, it is Invalid for all |
| | Dynamic Panels. |

Dynamic Panel Ordering can be further understood by the following numbered paragraphs:

Paragraph 1. A method for processing and analyzing a sample comprising: receiving by a system, the system including a processor coupled to a non-transitory computer readable medium, an assay definition file (ADF), the ADF enabling reporting rules configuration; receiving by the processor reporting rules from the user; sending by the processor control instructions to a sample processing bay which are not based on the reporting rules defined by the user; receiving by the processor scan data from the sample processing bay; sending by the processor scan data to an assay analysis module (AAM); receiving by the processor a detection report from the AAM based on the reporting rules defined by the user; and reporting by the processor the detection report.

Paragraph 2. The method of paragraph 2, wherein the reporting rules comprise at least a first target which has been selected for reporting.

Paragraph 3. The method of paragraph 2, wherein the reporting rules comprise at least a first target that has been selected for reporting and at least a second target that has not been selected for reporting.

Paragraph 4. The method of paragraph 2, wherein the scan data comprises at least a first scan data for a first target which has been selected for reporting and at least a second scan data for a second target that has not been selected for reporting.

Paragraph 5. The method of paragraph 2, wherein the scan data comprises at least a first scan data for a first target which has been selected for reporting and at least a second scan data for a second target that has not been selected for reporting and wherein the detection report comprises a detection result for the first target and does not include a detection result for the second target.

Paragraph 6. The method of paragraph 2, wherein the scan data comprises at least a first detection result for a first target which has been selected for reporting and at least a second detection result for a second target that has not been selected for reporting and wherein the detection report comprises a first detection result for the first target and does not include the second detection result for the second target.

Paragraph 7. A method for processing and analyzing a sample the method comprising storing by a non-transitory computer readable medium an assay definition file (ADF), the ADF enabling reporting rules configuration; receive reporting rules from a user or LIS; receive detection results from the sample processing bay; and analyze and report results of the detection results based upon the reporting rules.

Paragraph 8. The method for processing and analyzing a sample according to paragraph 7, wherein a first detection result is reported on a detection report and a second result is not reported on the detection report.

Paragraph 9. The method for processing and analyzing a sample according to paragraph 7, wherein a first detection result is “not detected” and it is reported on a detection report and a second result is “detected” and it is not reported on the detection report.

Paragraph 10. A method for processing and analyzing a sample comprising receiving by an instrument comprising an Instrument Software Module (ISW) at least one ADF file enabling reporting rules configuration; receiving user instructions defining reporting rules, wherein, when executed by the ISW, the apparatus performs steps comprising detection of a target analyte in the sample and reporting detection results for some but not all targets in the sample based on the reporting rules.

Paragraph 11. The method of paragraph 10, wherein the reporting rules define a first target that is reported on a detection report.

Paragraph 12. The method of paragraph 10, wherein the reporting rules define a first target that is reported on a detection report and define a second target that is not reported on a detection report.

Paragraph 13. The method of paragraph 10, wherein the reporting rules define a first target and second target that are reported on a detection report and the first target is detected and the second target is detected.

Paragraph 14. The method of paragraph 10, wherein the reporting rules define a first target that is reported on a detection report and a second target that is not reported on the detection report wherein the first target is detected and the second target is detected.

Paragraph 15. The method of paragraph 10, wherein the reporting rules define a first target that is reported on a detection report and a second target that is not reported on the detection report wherein the first target is not detected and the second target is detected.

Paragraph 16. The method of paragraph 10, further comprising updating a quality control rule when the instrument receives user instructions defining reporting rules.

Paragraph 17. The method of paragraph 10, further comprising updating a quality control rule when the instrument receives user instructions defining reporting rules wherein a first target is not reported on the detection report.

Paragraph 18. The method of paragraph 10, wherein enabling reporting rules configuration comprises some targets that are non-configurable.

Paragraph 19. The method of paragraph 10, wherein receiving user instructions defining reporting rules comprises receiving a first plurality of reporting rules and second plurality of reporting rules wherein the first plurality of reporting rules conflicts with the second plurality of reporting rules.

Paragraph 20. The method of paragraph 10, further comprising, receiving, by the instrument an operator selection of a first reporting rule.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method for creating a first Quality Control Rule on a detection instrument, the method comprising:
receiving by the detection instrument a user selection of a first installed assay;
receiving by the detection instrument the user selection of a first target from the first installed assay;
receiving by the detection instrument the user selection of a run frequency for the first target;
generating by the detection instrument a first QC test; and
processing by the detection instrument the first QC test according to the run frequency for the first target.

2. The method of claim 1, further comprising:
receiving by the detection instrument the user selection of a second target from the first installed assay;
receiving by the detection instrument the user selection of a run frequency for the second target;
receiving by the detection instrument the user selection of a QC test rotation order for the first target and second target; and
processing by the detection instrument the first QC test according to the run frequency and QC test rotation order.

3. The method of claim 2, wherein Days Until Due for the first QC test are automatically displayed by the detection instrument when the run frequency and/or QC test rotation order for the first QC test is generated.

4. The method of claim 1, wherein running the first QC test comprises obtaining a first QC test result wherein if the first QC test result does not match a first QC expected test result the detection instrument sends an alert.

5. The method of claim 1, further comprising:
receiving by the detection instrument the user selection of a second installed assay;
receiving by the detection instrument the user selection of a third target from the second installed assay;
receiving by the detection instrument the user selection of a run frequency for the third target, thereby generating a second QC test; and
processing by the detection instrument the second QC test according to the run frequency.

6. The method of claim 1, wherein processing the first QC test comprises obtaining a first QC test result, processing the second QC test comprises obtaining a second QC test result, and generating by the detection instrument a report comprising the first and second QC test results.

7. The method of claim 1 further comprising:
receiving by the detection instrument a user selection of a first installed assay from a QC menu;
receiving by the detection instrument a user instructions deselecting a first target from the first installed assay by the user;
receiving by the detection instrument the user selection of selecting a second target from the first installed assay by the user;
receiving by the detection instrument the user selection of a run frequency for the first target and second target by the user; and
processing by the detection instrument a second QC test according to the run frequency and ignoring a first QC test's run frequency.

8. The method of claim 1 further comprising:
providing by the detection instrument a QC scheduler drop-down wherein there is at least one installed assay and at least one installed QC Rule for the assay;
displaying by the detection instrument an assay drop down for assays having a QC Rule;
receiving by the detection instrument a user selection of an assay having a QC Rule;
displaying by the detection instrument an assay drop down comprising the QC Rules for the selected assay; and
displaying by the detection instrument a “create a new QC button” and a “manage a QC button”.

9. The method of claim 8, further comprising receiving by the detection instrument a user defined QC Accession ID for each QC Rule created.

10. The method of claim 8, further comprising receiving by the detection instrument a user defined comment for one or more QC Rule created.

11. The method of claim 8, further comprising updating by the detection instrument the QC Scheduler drop-down to reflect the frequency, rotation order, Lot Alert (on/off), Require Valid Control (yes/no) and/or Days Until Due parameters.

12. The method of claim 8, wherein displaying by the detection instrument an assay drop down comprising the QC Rules further comprising displaying the expected result for each assay target.

13. A diagnostic detection instrument based Quality support system for strengthening a healthcare provider's quality program comprising:
a diagnostic detection instrument having a processor, memory, and access to computer readable media;
a computer program stored on computer readable media having a quality assurance module; and
a user interface displayed on a monitor for interaction with the computer program wherein the quality assurance module is adapted to perform the method steps according to claim 1.

14. The diagnostic instrument of claim 13, further comprising a network connection between the diagnostic instrument and a computer network for allowing remote access to the computer program.

15. The diagnostic instrument of claim 13, wherein a pixel mapped calendar is stored on computer readable media and graphically displayed on a monitor having a plurality of colored geometries arranged in rows and columns for each date, each colored geometry representing status of a quality assurance task and being assigned a color corresponding to a current status of a quality assurance task.

16. The diagnostic instrument of claim 13, wherein a reminder function is stored on computer readable media and displayed on a monitor containing a visual representation of task compliance for each quality assurance task.

17.-100. (canceled)

101. The method of claim 1, further comprising:
managing quality control rules on a detection instrument, the managing comprising:
receiving by the detection instrument a user selection of a first QC test from a QC menu;
receiving by the detection instrument user modification instructions for the run frequency for the first QC test; and
processing by the detection instrument the first QC test according to the run frequency.

102. The method of claim 1, wherein a second Quality Control Rule is created for a second target from the first installed assay wherein the second Quality Control Rule and First Quality Control rule are different.

103. The method of claim 1, wherein a second Quality Control rule is created for a second target from the first installed assay wherein the run frequency for the first Quality Control Rule and a second run frequency for the second Quality Control rule are different.

104. A method for creating a Quality Control Rule on a detection instrument, the method comprising:
receiving by the detection instrument a user selection of a first installed assay;
receiving by the detection instrument the user selection of a target from the first installed assay;
receiving by the detection instrument the user selection of a second target from the first installed assay;
receiving by the detection instrument the user selection of a run frequency for a first target;
receiving by the detection instrument the user selection of a run frequency for the second target;
generating by the detection instrument a first QC test based on the first target;
receiving by the detection instrument the user selection of a QC test rotation order for the first target and second target; and
processing by the detection instrument the first QC test according to the run frequency and the QC test rotation order.
